SEQUENCE LISTING (119 sequences)

SEQ ID NO: 1 - protein, 8 aa, Artificial Sequence
  Synthetic sequence
ELANEIPV

SEQ ID NO: 2 - protein, 184 aa, Artificial Sequence
  Synthetic sequence
PFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPC

SEQ ID NO: 3 - protein, 188 aa, Artificial Sequence
  Synthetic sequence
PFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPKSRKHTVLNLKEPLYVGGAPDFSKLARAAAVSSGFDGAIQLVSLGGRQLLTPEHVLRQVDVTSFAGHPC

SEQ ID NO: 4 - protein, 4 aa, Artificial Sequence
  Synthetic sequence
KSRK

SEQ ID NO: 5 - protein, 167 aa, Artificial Sequence
  Synthetic sequence
EYLNAVTESEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPC

SEQ ID NO: 6 - protein, 175 aa, Artificial Sequence
  Synthetic sequence
EYLNAVTESELANEIPVEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPC

SEQ ID NO: 7 - protein, 189 aa, Artificial Sequence
  Synthetic sequence
DTLAFDGRTFVEYLNAVTESELANEIPVEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 8 - protein, 437 aa, Artificial Sequence
  Synthetic sequence
LGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESELANEIPVEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 9 - protein, 177 aa, Artificial Sequence
  Synthetic sequence
APVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGEC

SEQ ID NO: 10 - protein, 39 aa, Artificial Sequence
  Synthetic sequence
PPKPCDSQPCFHGGTCQDWALGGGFTCSCPAGRGGAVCE

SEQ ID NO: 11 - protein, 38 aa, Artificial Sequence
  Synthetic sequence
GDHPCLPNPCHGGAPCQNLEAGRFHCQCPPGRVGPTCA

SEQ ID NO: 12 - protein, 38 aa, Artificial Sequence
  Synthetic sequence
EKSPCQPNPCHGAAPCRVLPEGGAQCECPLGREGTFCQ

SEQ ID NO: 13 - protein, 40 aa, Artificial Sequence
  Synthetic sequence
AGHPCTRASGHPCLNGASCVPREAAYVCLCPGGFSGPHCE

SEQ ID NO: 14 - DNA, 582 bp, Artificial Sequence
  Synthetic sequence
catatggacaccctggcgttcgatggtcgtacctttgttgagtacctgaacgcggtgaccgagagcgaactggcgaacgagatcccggttgaaaaggcgctgcagagcaaccacttcgagctgagcctgcgtaccgaagcgacccaaggtctggtgctgtggagcggcaaagcgaccgaacgtgcggactacgttgcgctggcgattgtggatggtcacctgcagctgagctataacctgggcagccaaccggtggttctgcgtagcaccgttccggtgaacaccaaccgttggctgcgtgtggttgcgcaccgtgagcagcgtgaaggtagcctgcaagttggcaacgaagcgccggtgaccggtagcagcccgctgggtgcgacccagctggacaccgatggtgcgctgtggctgggtggcctgccggaactgccggttggtccggcgctgccgaaggcgtatggtaccggctttgtgggttgcctgcgtgacgtggttgttggtcgtcacccgctgcacctgctggaggatgcggttaccaaaccggaactgcgtccgtgcccgaccccgtaaggatcc

SEQ ID NO: 15 - DNA, 1326 bp, Artificial Sequence
  Synthetic sequence
catatgctgggtcgtgagggcaccttctgccaaaccgcgagcggtcaagatggtagcggtccgtttctggcggatttcaacggttttagccacctggaactgcgtggcctgcacaccttcgcgcgtgacctgggcgagaagatggcgctggaagtggtttttctggcgcgtggtccgagcggtctgctgctgtacaacggccagaagaccgacggtaaaggcgatttcgttagcctggcgctgcgtgaccgtcgtctggagtttcgttatgatctgggtaaaggtgctgcggttattcgtagccgtgagccggtgaccctgggtgcgtggacccgtgtgagcctggaacgtaacggtcgtaagggtgcgctgcgtgttggtgatggtccgcgtgtgctgggcgagagcccggttccgcacaccgtgctgaacctgaaggaaccgctgtacgttggtggcgcgccggacttcagcaagctggcgcgtgcggcggcggttagcagcggttttgatggcgcgattcagctggttagcctgggtggccgtcaactgctgaccccggagcacgtgctgcgtcaagttgacgttaccagctttgcgggtcacccgtgcacccgtgcgagcggtcatccgtgcctgaacggtgcgagctgcgttccgcgtgaagcggcgtacgtgtgcctgtgcccgggtggctttagcggtccgcactgcgagaagggtctggtggagaagagcgcgggtgacgtggataccctggcgttcgatggccgtacctttgttgagtatctgaacgcggtgaccgagagcgaactggcgaacgagatcccggttgaaaaggcgctgcagagcaaccacttcgagctgagcctgcgtaccgaagcgacccaaggtctggtgctgtggagcggcaaagcgaccgaacgtgcggactacgttgcgctggcgattgtggatggtcacctgcagctgagctataacctgggcagccaaccggtggttctgcgtagcaccgttccggtgaacaccaaccgttggctgcgtgtggttgcgcaccgtgagcagcgtgaaggtagcctgcaagttggcaacgaagcgccggtgaccggtagcagcccgctgggtgcgacccagctggacaccgatggtgcgctgtggctgggtggcctgccggaactgccggttggtccggcgctgccgaaggcgtatggtaccggctttgtgggttgcctgcgtgacgtggttgttggtcgtcacccgctgcacctgctggaggatgcggttaccaaaccggaactgcgtccgtgcccgaccccgtaaggatcc

SEQ ID NO: 16 - DNA, 19 bp, Artificial Sequence
  Synthetic sequence
gatgcacgcacctcgatgt

SEQ ID NO: 17 - DNA, 15 bp, Artificial Sequence
  Synthetic sequence
ggcccccctggcatt

SEQ ID NO: 18 - DNA, 22 bp, Artificial Sequence
  Synthetic sequence
gaccccagacaaatgtgatcct

SEQ ID NO: 19 - DNA, 18 bp, Artificial Sequence
  Synthetic sequence
ttcaggctcgggattcca

SEQ ID NO: 20 - DNA, 26 bp, Artificial Sequence
  Synthetic sequence
cctagtctattcatagctaatcaaga

SEQ ID NO: 21 - DNA, 22 bp, Artificial Sequence
  Synthetic sequence
agtggaggaaagctgtgcatac

SEQ ID NO: 22 - DNA, 20 bp, Artificial Sequence
  Synthetic sequence
aaagcgaccggcaatataaa

SEQ ID NO: 23 - DNA, 20 bp, Artificial Sequence
  Synthetic sequence
atagctcaggacccgatttg

SEQ ID NO: 24 - DNA, 21 bp, Artificial Sequence
  Synthetic sequence
acgcagctctgtgtcaaaggt

SEQ ID NO: 25 - DNA, 21 bp, Artificial Sequence
  Synthetic sequence
tgatgaacgagactcgacagc

SEQ ID NO: 26 - DNA, 20 bp, Artificial Sequence
  Synthetic sequence
gcgggagcgagcagatccag

SEQ ID NO: 27 - DNA, 20 bp, Artificial Sequence
  Synthetic sequence
ggaagctggcaaactggcag

SEQ ID NO: 28 - DNA, 21 bp, Artificial Sequence
  Synthetic sequence
tctcattgcaagatcatcgcc

SEQ ID NO: 29 - DNA, 21 bp, Artificial Sequence
  Synthetic sequence
ccccatgaataacacagcacc

SEQ ID NO: 30 - DNA, 22 bp, Artificial Sequence
  Synthetic sequence
ttagaggagaaatgccaaggaa

SEQ ID NO: 31 - DNA, 22 bp, Artificial Sequence
  Synthetic sequence
cactgatgaaggaggaaggaag

SEQ ID NO: 32 - DNA, 24 bp, Artificial Sequence
  Synthetic sequence
tggactctccggattgaccaagtt

SEQ ID NO: 33 - DNA, 24 bp, Artificial Sequence
  Synthetic sequence
atttgggatagttggctcctcgct

SEQ ID NO: 34 - DNA, 20 bp, Artificial Sequence
  Synthetic sequence
cccatctatcagcaggctcc

SEQ ID NO: 35 - DNA, 19 bp, Artificial Sequence
  Synthetic sequence
aaagcgataccgccttctg

SEQ ID NO: 36 - DNA, 23 bp, Artificial Sequence
  Synthetic sequence
taagaatgcttccaggaccactc

SEQ ID NO: 37 - DNA, 23 bp, Artificial Sequence
  Synthetic sequence
gctgtaacatctgccgatttttc

SEQ ID NO: 38 - DNA, 22 bp, Artificial Sequence
  Synthetic sequence
ctcggcatgaatctgatgaatg

SEQ ID NO: 39 - DNA, 20 bp, Artificial Sequence
  Synthetic sequence
tggcagttcccacgttattg

SEQ ID NO: 40 - DNA, 24 bp, Artificial Sequence
  Synthetic sequence
tggactatcagctccgctactatg

SEQ ID NO: 41 - DNA, 19 bp, Artificial Sequence
  Synthetic sequence
gtggcagtgttggtctcgc

SEQ ID NO: 42 - DNA, 20 bp, Artificial Sequence
  Synthetic sequence
ctacacgctacaccctcgac

SEQ ID NO: 43 - DNA, 20 bp, Artificial Sequence
  Synthetic sequence
acgtcctcacaccaggaaac

SEQ ID NO: 44 - DNA, 20 bp, Artificial Sequence
  Synthetic sequence
gcgcccaaaccgaagtcata

SEQ ID NO: 45 - DNA, 20 bp, Artificial Sequence
  Synthetic sequence
atgggggatgcaggattgag

SEQ ID NO: 46 - DNA, 22 bp, Artificial Sequence
  Synthetic sequence
ccgtatttctactcggatgctg

SEQ ID NO: 47 - DNA, 18 bp, Artificial Sequence
  Synthetic sequence
tggcttgggtttcctgtc

SEQ ID NO: 48 - DNA, 22 bp, Artificial Sequence
  Synthetic sequence
ggactcagctccatgtctatcc

SEQ ID NO: 49 - DNA, 22 bp, Artificial Sequence
  Synthetic sequence
actgaggtctcccctaagatcc

SEQ ID NO: 50 - DNA, 18 bp, Artificial Sequence
  Synthetic sequence
gagaaagtggttgagagg

SEQ ID NO: 51 - DNA, 18 bp, Artificial Sequence
  Synthetic sequence
gtaatcaacgacggcaag

SEQ ID NO: 52 - DNA, 23 bp, Artificial Sequence
  Synthetic sequence
tgacaggatgcagaaggagatta

SEQ ID NO: 53 - DNA, 19 bp, Artificial Sequence
  Synthetic sequence
agccaccgatccacacaga

SEQ ID NO: 54 - DNA, 20 bp, Artificial Sequence
  Synthetic sequence
ctataaattgagcccgcagc

SEQ ID NO: 55 - DNA, 20 bp, Artificial Sequence
  Synthetic sequence
gaccaaatccgttgactccg

SEQ ID NO: 56 - DNA, 20 bp, Artificial Sequence
  Synthetic sequence
acaccgtcctcaacctgaag

SEQ ID NO: 57 - DNA, 20 bp, Artificial Sequence
  Synthetic sequence
ccaggttgtagctcagttgc

SEQ ID NO: 58 - DNA, 20 bp, Artificial Sequence
  Synthetic sequence
cggccccgccatggagctcc

SEQ ID NO: 59 - DNA, 20 bp, Artificial Sequence
  Synthetic sequence
ggcagttaccgccaccgggg

SEQ ID NO: 60 - DNA, 20 bp, Artificial Sequence
  Synthetic sequence
agcatggacgtggctgtgcc

SEQ ID NO: 61 - DNA, 20 bp, Artificial Sequence
  Synthetic sequence
ggcgtgcgtgtgtagcctgt

SEQ ID NO: 62 - DNA, 20 bp, Artificial Sequence
  Synthetic sequence
aatggcacggactaatttgc

SEQ ID NO: 63 - DNA, 20 bp, Artificial Sequence
  Synthetic sequence
tatgagggtttgtggggtgt

SEQ ID NO: 64 - DNA, 21 bp, Artificial Sequence
  Synthetic sequence
aatgctgcagccccaaggaat

SEQ ID NO: 65 - DNA, 24 bp, Artificial Sequence
  Synthetic sequence
ctgggcaactggacaactcaactc

SEQ ID NO: 66 - DNA, 23 bp, Artificial Sequence
  Synthetic sequence
catcactgccacccagaagactg

SEQ ID NO: 67 - DNA, 23 bp, Artificial Sequence
  Synthetic sequence
atgccagtgagcttcccgttcag

SEQ ID NO: 68 - DNA, 20 bp, Artificial Sequence
  Synthetic sequence
gcctcaaaccttccaaatca

SEQ ID NO: 69 - DNA, 20 bp, Artificial Sequence
  Synthetic sequence
tggatccatttcctcaaagg

SEQ ID NO: 70 - DNA, 17 bp, Artificial Sequence
  Synthetic sequence
cctgtccaaactaaggc

SEQ ID NO: 71 - DNA, 19 bp, Artificial Sequence
  Synthetic sequence
ggttttctcatagatggcg

SEQ ID NO: 72 - DNA, 20 bp, Artificial Sequence
  Synthetic sequence
gtacctccaccatgccaagt

SEQ ID NO: 73 - DNA, 21 bp, Artificial Sequence
  Synthetic sequence
tcacatctgcaagtacgttcg

SEQ ID NO: 74 - DNA, 21 bp, Artificial Sequence
  Synthetic sequence
ccttccaggatgaggacatga

SEQ ID NO: 75 - DNA, 21 bp, Artificial Sequence
  Synthetic sequence
tgagtcacagaggatgggctc

SEQ ID NO: 76 - DNA, 22 bp, Artificial Sequence
  Synthetic sequence
cctccaactgtctaccagttcc

SEQ ID NO: 77 - DNA, 20 bp, Artificial Sequence
  Synthetic sequence
gcctggctaagttattgtgc

SEQ ID NO: 78 - DNA, 20 bp, Artificial Sequence
  Synthetic sequence
tgaatgcctgagagtcttgg

SEQ ID NO: 79 - DNA, 20 bp, Artificial Sequence
  Synthetic sequence
ttggcagcaaacagcttatc

SEQ ID NO: 80 - DNA, 18 bp, Artificial Sequence
  Synthetic sequence
tgctcgtggctgccttct

SEQ ID NO: 81 - DNA, 20 bp, Artificial Sequence
  Synthetic sequence
caggaagtgggagggtcaga

SEQ ID NO: 82 - DNA, 20 bp, Artificial Sequence
  Synthetic sequence
cgcccagacagaagtcatag

SEQ ID NO: 83 - DNA, 21 bp, Artificial Sequence
  Synthetic sequence
tcctcctttccaggtcagtta

SEQ ID NO: 84 - DNA, 20 bp, Artificial Sequence
  Synthetic sequence
tgcacccaaaccgaagtcat

SEQ ID NO: 85 - DNA, 21 bp, Artificial Sequence
  Synthetic sequence
ttgtcagaagccagcgttcac

SEQ ID NO: 86 - DNA, 20 bp, Artificial Sequence
  Synthetic sequence
caagctcatctgtgcagacc

SEQ ID NO: 87 - DNA, 22 bp, Artificial Sequence
  Synthetic sequence
cgcctgtagtgcataagagtcc

SEQ ID NO: 88 - DNA, 18 bp, Artificial Sequence
  Synthetic sequence
caccactccctgctgctt

SEQ ID NO: 89 - DNA, 18 bp, Artificial Sequence
  Synthetic sequence
acacttggcggttccttc

SEQ ID NO: 90 - DNA, 22 bp, Artificial Sequence
  Synthetic sequence
taggctggagatctacaagagg

SEQ ID NO: 91 - DNA, 20 bp, Artificial Sequence
  Synthetic sequence
agtgcttgaggtggttgtgg

SEQ ID NO: 92 - DNA, 20 bp, Artificial Sequence
  Synthetic sequence
agccttccgctaccttctta

SEQ ID NO: 93 - DNA, 20 bp, Artificial Sequence
  Synthetic sequence
gctgttgcctttgttcttgg

SEQ ID NO: 94 - DNA, 24 bp, Artificial Sequence
  Synthetic sequence
cgtgtggatacaggatgttgacag

SEQ ID NO: 95 - DNA, 24 bp, Artificial Sequence
  Synthetic sequence
aggaggagcccatctttctgtaac

SEQ ID NO: 96 - DNA, 22 bp, Artificial Sequence
  Synthetic sequence
ggttcacgatcctgctcctgac

SEQ ID NO: 97 - DNA, 22 bp, Artificial Sequence
  Synthetic sequence
gctgggcctgagaacactcaag

SEQ ID NO: 98 - DNA, 20 bp, Artificial Sequence
  Synthetic sequence
tgagcaagagaggccctatc

SEQ ID NO: 99 - DNA, 20 bp, Artificial Sequence
  Synthetic sequence
aggcccctcctgttattatg

SEQ ID NO: 100 - DNA, 21 bp, Artificial Sequence
  Synthetic sequence
gcctcttctcattcctgcttg

SEQ ID NO: 101 - DNA, 20 bp, Artificial Sequence
  Synthetic sequence
ctgatgagagggaggccatt

SEQ ID NO: 102 - DNA, 20 bp, Artificial Sequence
  Synthetic sequence
acggccttccctacttcaca

SEQ ID NO: 103 - DNA, 20 bp, Artificial Sequence
  Synthetic sequence
catttccacgatttcccaga

SEQ ID NO: 104 - DNA, 21 bp, Artificial Sequence
  Synthetic sequence
cgtgcgtgacatcaaagagaa

SEQ ID NO: 105 - DNA, 20 bp, Artificial Sequence
  Synthetic sequence
tggatgccacaggattccat

SEQ ID NO: 106 - DNA, 25 bp, Artificial Sequence
  Synthetic sequence
ccuuugucgaguaccucaacgcugu

SEQ ID NO: 107 - DNA, 25 bp, Artificial Sequence
  Synthetic sequence
acagcguugagguacucgacaaagg

SEQ ID NO: 108 - DNA, 25 bp, Artificial Sequence
  Synthetic sequence
cauacggcaacgagugucagcugaa

SEQ ID NO: 109 - DNA, 25 bp, Artificial Sequence
  Synthetic sequence
uucagcugacacucguugccguaug

SEQ ID NO: 110 - DNA, 25 bp, Artificial Sequence
  Synthetic sequence
gcgauuuauggacuuugacugguuu

SEQ ID NO: 111 - DNA, 25 bp, Artificial Sequence
  Synthetic sequence
aaaccagucaaaguccauaaaucgc

SEQ ID NO: 112 - DNA, 25 bp, Artificial Sequence
  Synthetic sequence
ggagaccugccaguuuaacucugua

SEQ ID NO: 113 - DNA, 25 bp, Artificial Sequence
  Synthetic sequence
uacagaguuaaacuggcaggucucc

SEQ ID NO: 114 - DNA, 25 bp, Artificial Sequence
  Synthetic sequence
agguucccuucaggugggcaaugaa

SEQ ID NO: 115 - DNA, 25 bp, Artificial Sequence
  Synthetic sequence
uucauugcccaccugaagggaaccu

SEQ ID NO: 116 - DNA, 25 bp, Artificial Sequence
  Synthetic sequence
ccaaaguccugugauucccagccuu

SEQ ID NO: 117 - DNA, 25 bp, Artificial Sequence
  Synthetic sequence
aaggcugggaaucacaggacuuugg

SEQ ID NO: 118 - DNA, 25 bp, Artificial Sequence
  Synthetic sequence
ggagcucacggagacuucaacuauu

SEQ ID NO: 119 - DNA, 25 bp, Artificial Sequence
  Synthetic sequence
aauaguugaagucuccgugagcucc

(19) United States
(12) Patent Application Publication
(10) Pub. No.: US 2023/0257435 A1
(43) Pub. Date: Aug. 17, 2023

(54) Wound Healing Composition and Uses Thereof

(71) Applicant: AGENCY FOR SCIENCE, TECHNOLOGY AND RESEARCH, Singapore (SG)

(72) Inventors: Sayan Chakraborty, Singapore (SG); Wanjin Hong, Singapore (SG)

(21) Appl. No.: 18/014,449

(22) PCT Filed: Jun. 23, 2021
(86) PCT No.: PCT/SG2021/050363

§ 371 Date: Jan. 4, 2023

(30) Foreign Application Priority Data
Jul. 24, 2020 (SG) 10202007138Y

(51) Int. Cl. C07K 14/47; A61P 17/02

(52) U.S. Cl. CPC C07K 14/4725; A61P 17/02; A61K 38/00

(57) ABSTRACT

The present disclosure provides a pharmaceutical composition comprising an Agrin fragment or derivative thereof, and uses of the pharmaceutical composition. The present disclosure also provides method of producing the pharmaceutical composition.

### CROSS-REFERENCE TO RELATED APPLICATIONS

This application claims the benefit of priority of Singapore provisional application No. 10202007138Y, filed 24 Jul. 2020, the contents of it being hereby incorporated by reference in its entirety for all purposes.

### FIELD OF THE INVENTION

The present invention relates generally to the fields of molecular biology and biochemistry. In particular, the present invention relates to compositions for the treatment of wounds.

### BACKGROUND OF THE INVENTION

It is estimated that over 400 million people globally have diabetes (http://www.idf.org). In Singapore alone it is estimated that 11% of 20-80-year-olds have diabetes, second only to the United States, and this number is set to rise significantly.

A major complication of diabetes is non-healing wounds. What can start as a small scratch, can later become a large, non-healing wound that in some cases can only be treated with limb amputation. In Singapore, non-healing wounds are the leading cause of non-traumatic lower limb amputations with more than 4 lower limb amputations occurring daily. Non-healing wounds or ulcers can persist for 12 months or longer and have a very high recurrence rate of 65%. It is estimated that 33% of the annual diabetes budget is spent on diabetic foot ulcers. The inability to effectively treat non-healing wounds has resulted in dramatically increased wound care costs in Singapore in recent years, with current estimates being well over S$700 million annually.

Despite their prevalence and the significant healthcare burden of non-healing wounds, there remains no effective treatment. Thus, there is an unmet need for providing compositions and methods for treating non-healing wounds.

### SUMMARY OF THE INVENTION

In one aspect, there is provided a pharmaceutical composition comprising an Agrin fragment or derivative thereof, wherein the Agrin fragment or derivative thereof comprises the LG3 domain of Agrin and an eight-amino-acid insert ELANEIPV (SEQ ID NO: 1) at the z-site of the LG3 domain.

In another aspect, there is provided a vector comprising the nucleic acid molecule encoding for an Agrin fragment or derivative thereof, wherein the Agrin fragment or derivative thereof comprises the LG3 domain of Agrin and an eight-amino-acid insert ELANEIPV (SEQ ID NO: 1) at the z-site of the LG3 domain.

In another aspect, there is provided a host cell comprising the vector as disclosed herein.

In another aspect, there is provided a hydrogel or scaffold comprising the pharmaceutical composition as disclosed herein.

In another aspect, there is provided the pharmaceutical, the hydrogel, or the scaffold as disclosed herein, for use in therapy.

In another aspect, there is provided a method of treating a wound, the method comprising administering a pharmaceutically effective amount of the pharmaceutical composition, the hydrogel, or the scaffold as disclosed herein, to a subject in need thereof.

In yet another aspect, there is provided use of a pharmaceutically effective amount of the pharmaceutical composition, the hydrogel, or the scaffold as disclosed herein, in the manufacture of a medicament for the treatment of wound.

### BRIEF DESCRIPTION OF THE DRAWINGS

The invention will be better understood with reference to the detailed description when considered in conjunction with the non-limiting examples and the accompanying drawings, in which:

FIG. 1 shows results of assays investigating the effect of skin injury on the expressions of various proteins, including Agrin. (a) Gene-expression analysis of an ECM-based wound signature from single-cell transcriptomics of wounded cells in a wound-healing mouse model (Students ‘t’ test, *p<0.05, **p<0.005, respectively). (b) Confluent cells were wound-scratched and allowed to migrate. After indicated time-points, mRNA isolated from migrating cells were analyzed by RT-PCR for the indicated genes. Data represented as mean+/−s.d. Multiple t tests were performed for all the genes comparing the respective phases post-wounding; p values represented in tabular form (values in grey signify reduction). (c) Western blot analysis of migrating skin cells (as in panel b) for Agrin expression. GAPDH was used as loading controls. Densitometric quantification of Agrin levels in the indicated cells is shown graphically. Data represented as mean+/−s.d. (n=3 experiments, Multiple t tests, p values shown in the figure) (d) Western blot analysis in non-wounded skin keratinocytes and fibroblasts for the indicated proteins. GAPDH was used as loading controls. Densitometric quantification of Agrin levels in the indicated cells is shown graphically (n=3 experiments, **p=0.001, *p=0.02, Students ‘t’ test). (e) Confocal microscopy sections of mice skin punch-wound biopsies at indicated days was analyzed for Agrin immunofluorescence and counterstained with DAPI. Scale bar: 50 μm. Mean fluorescence intensity (MFU) of Agrin staining in the mice skin sections quantified graphically. Data represented as mean+/−s.d. (n=3 mice, three sections analyzed for each time, **p=0.003, students ‘t’ test). The white dashed line denotes the epidermis and dermis boundaries, respectively. (f) Immunohistology sections of mouse skin punch wound biopsies at indicated day post-injury showing relative Agrin expression. Scale bar: 100 μm, EP-Epidermis, D-Dermis, d-WAT-Dermal White adipose tissue. The dashed line denotes epidermal and dermal boundary. Mean Agrin intensity in the mice skin sections quantified graphically by ImageJ. Data represented as mean+/−s.d. (n=3 mice, three sections analyzed for each time-point, ANOVA and Students ‘t’ tests, p values represented in the figure). The results show that Agrin expression is triggered during mechanical injury to skin.

FIG. 2 are assays showing effects of AGRN and GPC1 knockdown on keratinocyte migration post-wounding. (a) RT-PCR analysis showing the knockdown of AGRN and GPC1 in HaCaT cells. (b) Control, Agrin depleted, and GPC1 knockdown cells were subjected to scratch wound assays. Representative bright-field images showing relative migration are presented at the indicated time-points. The mean non-migrated area+/−s.d. was quantified using ImageJ (n=3, Students ‘t’ test, **p=0.005, ****p=0.000009, ****p=0.00002, respectively). Scale bar: 50 μm. (c) Comparisons of migration velocities of Agrin and GPC1 knockdown HaCaTs post-wound scratch over a period of 24 h (n=3, Students ‘t’ test, ****p<0.00001). The results show that Agrin and Glypican-1 play roles in keratinocyte migration post-wounding.

FIG. 3 are assays showing the effects of Agrin knockdown on wound healing in vivo. (a) Scheme showing the Agrin siRNA treatments prior to wounding and the application of siRNA containing Aquaphore ointment on wounded mice skins. (b) Western blot on mice punch-biopsy wounds lysates after 72 h post-intradermal siRNA injections at day 0 of wound injury in non-splinted (left panel) and splinted (right) conditions. The mice's skin lysates were probed for Agrin. Actin or GAPDH served as loading controls. (c) Confocal microscopy images showing Agrin immunostaining in wounded mice skins treated with control and indicated Agrin siRNAs at day 5 post-injury in non-splinted wounds. Scale bar: 50 μm. The white dashed line indicates the migrating epidermis. (d) Hematoxylin and Eosin stained mice skin sections treated with control and Agrin siRNA #1 at day 8 post-injury (n=4 mice per group). The two-directional arrow refers to the uncovered wound area. The asterisk * denotes the migrating epithelial tongue covering the wound. Scale bar: 50 μm. (e) Western blot verifying Agrin knockdown by the indicated stealth siRNAs against human Agrin in HEK cells. GAPDH served as a loading control. (f) Confocal microscopy images showing Agrin immunostaining in human skin explants treated with Control, Agrin siRNA #1. One batch of Agrin siRNA treated explants were subsequently rescued with sAgrin from day 2 onwards. The images are shown at day 8 post-wounding. Nuclei are stained with DAPI. Arrows indicate the wound area left uncovered. Scale bar: 50 μm. (g) Representative Hematoxylin and Eosin stained sections of human skin explants treated with either 75 nM Control or Agrin siRNA #1 at day 8 post-injury. The third panel consists of skin explant sections that were treated with Agrin siRNA #1 ointment on day 0, but subsequently received ointments that additionally contained sAgrin (20 μg) on days 2, 4 and 6, respectively. The arrow indicates the migrating epithelial tongue covering the wound area in control skin sections. The double-direction arrow refers to the uncovered wound areas. Scale bar: 500 μm. The results show that silencing Agrin delays in vivo wound healing.

FIG. 4 shows the results of assays investigating the effect of Agrin depletion on skin wound healing. (a) Mice skin were treated with scrambled or Agrin siRNA #1-3. Three days after, the siRNA treated regions were punch wounded and subsequently treated with Aquaphore-based topical ointment containing 75 nM indicated siRNAs every two-three days, respectively. A representative photograph of punch wounds at the indicated days of mice skin treated with aforementioned siRNA containing topical ointment is shown at the indicated days post-wounding. Average wound diameter was plotted graphically on indicated days post-wounding. Data represented as mean+/−s.d (n=4 mice per group, two-way ANOVA, *p=0.02, 0.03 and 0.01, respectively). Scale bar: 1 mm. (b) Confocal microscopy images showing K17 and DAPI staining in the migratory epithelial tongue at day 5 post-wounding in the mice skin sections treated with the indicated siRNAs. Scale bar: 50 μm. Relative K17 staining intensity represented as mean+/−s.d. (n=3 sections per group, Students ‘t’ test, p=0.01, p=0.002, respectively). (c) Photographs showing splinted wound dressing scheme employed in ICR mouse strains. The wound diameter quantified at day 7 in splinted (closed) and non-splinted (open) wounds are quantified (n=3 mice in each group, *p=0.05, Students ‘t’ test). (d) Mice skin were treated with scrambled or Agrin siRNA #1. Three days after, the siRNA treated regions were punch wounded and subsequently treated with Aquaphore-based topical ointment containing 75 nM indicated siRNAs every two-three days, respectively. The wounds tethered with a 10 mm splint were covered with Tegaderm. Representative photographs are shown at indicated days of dressing and siRNA ointment applications. Average wound diameter was plotted graphically on indicated days post-wounding. Data represented as mean+/−s.d (n=3 mice per group, ANOVA p=0.003). (e) Confocal microscopy images showing K17 and DAPI staining in the migratory epithelial tongue at day 7 post-wounding in the mice skin sections treated with the indicated siRNAs. Scale bar: 50 μm. White block arrow indicates the attenuated epithelial tongue. Relative K17 occupancy represented as mean+/−s.d. (n=3 sections per group, Students ‘t’ test, p=0.001). (f) Picrosirius red staining showing the relative collagen distribution in the wound beds of control and Agrin siRNA treated mouse skin at day 7. Representative bright-field and polarized light images are shown. Boxed area represents the enlarged view. Scale bar: 100 μm. The relative collagen fibers were quantified using ImageJ (n=4 sections analyzed from 3 mice per group, Students ‘t’ test, *p=0.02). (g) Human skin explants were punch wounded and treated with ointments containing either 75 nM scrambled control or Agrin siRNA #1 every three days. For panel 3, 20 μg sAgrin was incorporated in the ointment after day 2 and applied every two days, respectively. Representative photograph of skin wounds at days 0 and 10, respectively, are shown. Relative wound diameters were calculated at day 10 post-injury are presented as mean+/−s.d. (n=3 per group, students ‘t’ test, ***p=0.0001, *p=0.03, respectively). Scale bar: 0.5 mm. (h) Representative confocal images showing K17 staining in the migrating keratinocytes covering the wound beds of control, Agrin depleted and those rescued by sAgrin skin explants. The arrow marks the leading edge of migratory keratinocytes. The white line indicates the wound boundary. Scale bar: 50 μm. The results show that Agrin depletion impairs skin wound healing.

FIG. 5 are assays showing effects of Agrin knockdown on 2D migration of keratinocytes and dermal fibroblasts. (a) RT-PCR analysis for Agrin gene expression in BJ and HaCaT cells treated with either control or Agrin siRNA. Data presented as mean+/−s.d. These knockdown cells were subsequently used for transwell migration assays. Migration was analyzed for a batch of Agrin depleted cells in response to sAgrin (10 μg/ml) in the bottom chambers as a chemoattractant (third panels). Migrating were quantified using ImageJ and represented as mean+/−s.d. (n=3, Students ‘t’ test, p=0.0004, 0.0003, and 0.0005, respectively). Scale bar: 20 μm. (b) Control, Agrin depleted, and Agrin knockdown BJ or primary mouse dermal fibroblasts (DF) cells pre-treated with 10 μg/ml sAgrin for 18 h were subjected to scratch wound assays. Representative bright-field images showing relative migration are presented at the indicated time-points. The mean non-migrated area+/−s.d. was quantified using ImageJ (n=3, Students ‘t’ test, *p<0.05, **p<0.004, ***p<0.0005, ****p=0.000002, ****p<0.000001, respectively). Scale bar: 20 μm. A Western blot verifying the knockdown of Agrin in DF is also shown with GAPDH as loading control. (c) Scratch wound assay in control, Agrin depleted, and Agrin knockdown indicated keratinocytes pre-treated with 10 μg/ml sAgrin for 18 h. Representative brightfield images are shown. Scale bar: 20 μm. RT-PCR analysis verifying Agrin expression in HEK cells is also presented as mean+/−s.d (middle panel), while a Western blot confirming Agrin knockdown is presented for primary mouse keratinocytes (rightmost panel). Results are quantified as in panel (b) (n=3, Students ‘t’ test, ****p<0.000001, **p<0.005, *p<0.05, respectively, all other p values are represented in the panels). (d) Comparisons of migration velocities of indicated cells treated as in panels (b and c) at 15 h post-wounding (n=4 independent migration events, Students ‘t’ test, p values indicated in the figure). The results show that Agrin knockdown hampers 2D migration of keratinocytes and dermal fibroblasts.

FIG. 6 Agrin impacts keratinocyte proliferation following wound injury. (a) Control, Agrin depleted indicated keratinocytes and those rescued with 10 μg/ml sAgrin for 18 h were labelled with 20 μM BrdU for 12 hours, before proceeding with wound-scratch assays. The cells were fixed at 4 hours post-scratching and stained with an Anti-BrdU and K17 antibodies. Nuclei were counterstained with DAPI. Representative confocal images at the wound edge (leader) and at-least 200 μm away from the wound margin (followers) are shown. The relative percentage of proliferating cells are shown for each condition graphically (n=5-6 images analyzed for each conditions from three replicates, Students ‘t’ test, **p=0.006, *p-0.003, p=0.005, ***p=0.0001, respectively). Scale bar: 10 μm. (b-c) Schematic showing the states of proliferation at the migrating wound margins. Immunohistological sections stained for Ki67 in control and Agrin depleted wound edges and beds at day 7 post-wounding. Filled arrows denote the base of epidermis (Ep), while arrows represent the wound margin. Asterisk (*) denotes the loss of Ki67 proliferation at the wound margins. Scale bar: 100 μm. The percentage of proliferating cells was quantified using Image J (n=4 sections analyzed from two individual mice, Students ‘t’ tests, **p=0.001). The results show that Agrin impacts keratinocyte proliferation following wound injury.

FIG. 7 shows results of assays investigating whether Agrin generates a mechanically competent environment favoring collective keratinocyte migration and wound closure. (a) Confluent treated HaCaT cells were cultured in soft substrates alone or those supplemented with sAgrin (10 μg/ml) for 18 h. Post-removal of barrier stencil, the collective migration of cells was imaged under 4× bright-field microscope at 0 h and 24 h, respectively. Untreated HaCaTs cultured on stiff substrates were used as controls. Representative wound area collectively covered by HaCaT under different ECM rigidities are shown as mean+/−s.d. (n=3, students ‘t’ test, **p<0.001). Scale bar: 50 μm. (b) HaCaT cells either left untreated or treated with sAgrin (10 μg/ml) was cultured in soft substrates and allowed to migrate as in (a). The cells were fixed after 4 h post-removal of barrier and stained with a K17. Representative confocal images showing K17 and DAPI staining in the migrating leading edges. Scale bar: 10 μm. The relative K17 intensity was quantified to denote the leader cell fractions in untreated and sAgrin treated HaCaTs at 4 h post-migration (n=10 images per group, Students ‘t’ test, *p=0.01). White arrows indicate the leader cells. Dashed line represents the wound edge. (c) Control and Agrin depleted HaCaTs were grown in stiff substrates alone for 18 h, before analyzing their migrating potential at 0 h and 24 h post-removal of barrier stencil. One batch of Agrin depleted cells were cultured on stiff substrates that contained 10 μg/ml sAgrin for 18 h and subsequently analyzed for migration as above. Representative bright-field images and quantified migratory area covered by each conditions of cells are shown as mean+/−s.d. Scale bar: 50 μm. Western blot showing the knockdown of Agrin in the HaCaT cells. GAPDH served as loading controls. (d) Cells treated same as in (c) were fixed at 4 h post-removal of barrier and stained for K17 and DAPI. Representative confocal images are shown. Scale bar: 10 μm. The dashed line represents the wound edge. Relative percentage of leader cells expressing K17 are quantified and represented as mean+/−s.d. (n=10 images per condition, Students ‘t’ test, **p=0.002 and 0.001, respectively). (e) 3D-stiffness-dependent migration assays showing the relative migration of primary mouse keratinocytes over dermal fibroblasts at days 0 and 5 post-wounding in the presence or absence of 10 μg/ml sAgrin in soft matrix (0.8 KPa). Representative bright-field images of migrating cells are shown. Scale bar: 100 μm. The relative migration area was quantified by ImageJ (n=3 images per group, Students ‘t’ test, *p=0.02). Black dotted line represents the original wound region. (f) Representative bright-field images of control and Agrin depleted primary mouse keratinocytes and dermal fibroblasts migrating either alone or in the presence of 10 μg/ml sAgrin embedded in stiff matrix (30 KPa) at indicated days. Scale bar: 100 μm. The relative migration area was quantified by ImageJ (n=3 images per group, Students ‘t’ test, *p=0.05, **p=0.005, **p=0.002, and ***p=0.0005, respectively). Western blot images reveal the degree of Agrin knockdown in indicated cells. GAPDH was used as loading controls. (g) Schematic showing the extraction of skin from day 3 old mouse pup that is subsequently cultured in soft or stiff ECMs alone or those containing sAgrin, respectively. (h) Bright-field microscopy images showing keratinocyte outgrowth from mouse skin explants on soft ECM alone or supplemented with 20 μg/ml sAgrin on day 5. Outgrowth area was measured by ImageJ and represented graphically as mean+/−s.d. (n=3 explants per group, Students ‘t’ test, *p=0.03). Scale bar: 50 μm. (i) Mouse skin explants cultured on stiff ECM were treated with mouse control or Agrin siRNAs for 3 days. One batch of Agrin depleted mouse explants were subsequently cultured on stiff ECM conditioned with 20 μg/ml sAgrin for an additional 2 days. Representative RT-PCR analysis for Agrin expression at day 5 is shown as mean+/−s.d. (n=3 mice explants, per group). Bright-field images showing keratinocyte outgrowth are also shown. Scale bar: 50 μm. Outgrowth area was quantified using ImageJ and presented as mean+/−s.d. (n=3 explants per group, Students ‘t’ test, *p=0.019 and 0.04, respectively). (j) AFM measuring cell stiffness in migrating Control, Agrin depleted and those rescued with 10 μg/ml sAgrin at 4 hours post-wounding. Relative stiffness in Pascals are shown (n=10-15 cells analyzed per group, three replicates, Students ‘t’ test, *p=0.01, *p=0.008, respectively). (k) Heatmap showing traction forces in control, Agrin depleted and rescued HaCaTs migrating on microfabricated substrates. White arrows point towards the leading edge. Scale bar: 100 μm. Computation of leading edge traction forces across all conditions (n=6-10 images from three experiments, Students ‘t’ test, *p=0.02 and 0.04, respectively. (1) PIV analysis showing velocity vectors in migrating control, Agrin depleted and rescued HaCaTs on PDMS substrates. Green arrows represent velocity fields. White asterisks represent areas of vortex. Scale bar: 100 μm. The quantified velocities are presented graphically (n=3, Students ‘t’ test, ***p=0.0001). The results show that Agrin sensitizes collective migration under ECM rigidity by enhancing traction stress and fluidic velocity dynamics.

FIG. 8 shows effects of Agrin on stiffness of migrating keratinocytes. (a) Control, Agrin depleted, and Agrin knockdown primary mouse keratinocytes cells pre-treated with 10 μg/ml sAgrin for 18 h were subjected to scratch wound assays. At 4 h post-scratching, the cells at the leading edge (white line) were analyzed by AFM. Representative Western blot verifying Agrin knockdown is shown with GAPDH as a loading control. Scale bar: 10 μm. (b) AFM stiffness map of indicated conditions are shown. Force scale represents 0-7 KPa. The results show that Agrin attributes stiffness to migrating keratinocytes.

FIG. 9 shows results of assays investigating whether Agrin tunes cellular mechanics during wound injury via coordinating cytoskeletal architecture. (a) Confocal images of wound edges of confluent control, Agrin depleted and those treated with 10 μg/ml sAgrin for 18 h at the indicated time-points post-injury. The cells were immunostained for phosphor-myosin light chain (pMLC) and F-Actin and counterstained with DAPI. Scale bar: 10 μm. (b) Confluent cultures of control, Agrin siRNA-treated and Agrin depleted HEK cells treated with 10 μg/ml sAgrin for 18 h were scratched. Cell lysates from the migrating area were collected at the indicated time-points and analyzed for pMLC activation by Western blot. Agrin expression verified the knockdown efficacy while Actin served as a loading control. (c-d) Confluent HEK cells were either left untreated or nourished with 10 μg/ml sAgrin for 18 h. One batch of sAgrin nourished HEK cells were further subjected to 10 μm Blebbistatin for 2 h before wound scratch and cells were allowed to migrate for the indicated time either alone or in presence of sAgrin and Blebbistatin. Following scratch-wounding, the cells were fixed at the indicated time-points and stained for pMLC, F-Actin and DAPI. Representative confocal images are shown. Scale bar: 10 μm (c). For panel d, migrating cells were fixed at 18 h post-wounding and visualized under a brightfield microscope. The relative wound area migrated by cells of each condition is represented as mean+/−s.d. (n=3, Students ‘t’ test, **p=0.001, ***p=0.0001 respectively). (e) Control and Agrin siRNA #1 treated HEK cells were coated with 4.5 μm magnetic beads conjugated with 20 μg BSA or sAgrin for 30 min at 4° C. The confluent cells were then scratched and allowed to migrate for 30 min in the presence or absence of a permanent magnet applying force. At 30 min post-wounding, cells were fixed and immunostained for pMLC and Actin with DAPI visualizing the nuclei. Representative confocal images for sAgrin coated beads are shown. Scale bar: 10 μm. White circles indicate beads. White arrows indicate enriched pMLC-Actin structures. (f) Quantification of pMLC intensity in vicinity of sAgrin beads in Agrin depleted HEK cells as mean intensity from three independent experiments (n=8-15 cells were analyzed, Students ‘t’ test, *p=0.04, ***p=0.0001, respectively. Scale bar: 10 μm. White circle refers to the beads, while the blue dashed circle represents the region of interest (ROI) 200μ² area quantified around each bead. (g) HEK cells were cultured in small (800μ²) or large (1600 μ2) fibronectin-coated crossbow patterns alone or those coated with 20 μg/ml sAgrin for 4-6 h. Representative confocal images of crossbow shaped cells immunostained for pMLC and F-actin and counterstained with DAPI are shown. The relative number of F-actin stress fibers are quantified as mean+/−s.d. from three independent experiments (n=10 cells per group, Students ‘t’ test, **p=0.005, *p=0.01, respectively). Scale bar: 10 μm. (h) Confocal images of pMLC intensity map of HEK cells plated for 4 h in large fibronectin or fibronectin-sAgrin coated micropatterns. Scale bar: 10 μm. Quantification of stress curvature of transverse arcs of crossbow shaped cells is presented as mean+/−s.d. from three experiments (n=10-15 cells per group, Students ‘t’ test, **p=0.002). (i) Control or Agrin depleted HEK cells were plated on fibronectin patterns or on fibronectin and sAgrin coated large micropatterns for 4 h. The resulting crossbow shaped cells were fixed and immunostained with pMLC and F-actin with DAPI marking the nuclei. Representative confocal images are shown. Scale bar: 10 μm. Stress curvature indicated by pMLC intensity at the respective transverse arcs of crossbow cells were quantified as mean+/−s.d. from three experiments (n=10 cells per group, Students ‘t’ test, **p=0.001, **p=0.005, respectively). The results show that Agrin tunes cell mechanics during wound injury.

FIG. 10 shows effects of Agrin-induced force recognition on actomyosin engagement. (a) Set-up of magnetic force transmission to wounded keratinocytes. Briefly, keratinocytes (HaCaT) were coated with pre-conjugated magnetic beads with either control or sAgrin for 30 min at 4° C. The cells were subsequently scratched and allowed to migrate at 37° C. either under standard culture conditions or under a permanent magnet placed 6 mm above the cells that exerted a force of 200 pN for 30 min. (b) SDS-PAGE gel showing the respective conjugation of 20 μg ligands—sAgrin, Bovine Serum Albumin (BSA) and Fibronectin (FN) to magnetic beads. The respective free proteins (probed in the supernatants before incubation with beads) are diminished in the bead-free supernatants after covalent conjugation. (c) HaCaT cells coated with BSA, sAgrin or FN beads for 30 min, were allowed to migrate under standard conditions or a permanent force for an additional 30 min. The cells were subsequently fixed and stained for active Integrin β1 (HUTS-4 clone) and Agrin. Nuclei were counterstained with DAPI. The white circles denote the bead location. Scale bar: 10 μm. The relative intensity at an area of 200μ² around each bead was quantified from three experiments and presented as mean+/−s.d. (n=10-15 cells per group). (d-e) Control and Agrin siRNA #1 treated HEK cells were subjected to force transduction as in (b) with sAgrin and FN (d) or syndecan 4 beads (e) after 72 h post-siRNA treatments. The beads adhered proteins were magnetically separated and were resolved in a Western blot analysis probing for pMLC and total MLC. Respective cell lysates were probed for Agrin and GAPDH served as a loading control. (f-h) Representative confocal images of Agrin depleted HEK cells being probed with sAgrin (f), FN (g) or BSA (h) coated magnetic beads under standard conditions (no force) or application of permanent force. Cells were fixed and immunostained for pMLC and F-actin, respectively. DAPI marked the nuclei. White circles represented the beads. Scale bar: 10 μm. (i) Agrin depleted HaCaT cells were exposed with increased concentrations of sAgrin-coated magnetic beads for 30 min. Following wounding, the cells were allowed to migrate for 30 min under no force or permanent force applications and subsequently fixed and stained for pMLC. The nuclei were stained with DAPI. The block arrows represent enhanced pMLC recruitment near beads. The pMLC intensity presented as mean+/−s.d. in a 200μ² area around the beads was quantified from three experiments (n=10-15 cells). Scale bar: 10 μm. (j) Agrin depleted HaCaT were treated with sAgrin beads and allowed to migrate under sustained force applications for the indicated time-points. The resultant cells were treated and quantified as in (i). The results show that force recognition by Agrin to wounded keratinocytes engages actomyosin.

FIG. 11 shows results of transcriptome analysis of Agrin depleted keratinocytes. (a) RNA-Seq revealing a loss of Agrin gene expression (n=3 replicates). MDS plot of bulk population of RNA-Seq from siControl and siAgrin HaCaTs (n=3 samples). (b) Gene ontology (GO) analysis in Agrin knockdown HaCaT cells showing the most significantly down-regulated gene clusters. (c) GSEA analysis upon Agrin knockdown reveals that gene sets belonging to the ECM structural constituents are highly down-regulated. (d) Up- and down-regulated gene clusters of NABA matrisome (associated with ECM) upon Agrin depletion in HaCaT cells. (e) RT-PCR analysis validating a selected set of significantly up- and down-regulated genes.

FIG. 12 shows results of assays identify the downstream effectors of Agrin mediated mechanotension in keratinocytes upon wound injury. (a) Volcano plot showing the differentially expressed genes in control and Agrin depleted HaCaTs cultured in stiff plastic plates. Inset showing the levels of MMPs. (n=3 replicates) (b) GSEA analysis showing the down-regulated cluster corresponding to genes that mediate positive regulation to wounding. (c) HaCaT cells plated on soft substrates were treated with increasing concentrations of sAgrin for 18 h (left panel) or treated with 10 μg/ml sAgrin for the indicated time (right panel). Western blot analysis was performed to detect MMP12 and Agrin levels. GAPDH served as loading controls. (d) Control or Agrin siRNA treated HaCaT cells were plated on stiff substrates. Batches of Agrin depleted cells were further plated on stiff substrates containing increasing concentrations of sAgrin and cultured for 18 h. Subsequently, cell lysates were analyzed by Western blotting for the indicated proteins. Actin served as a loading control. The densitometric results of three independent experiments for MMP12 were quantified as mean+/−s.d. (Students ‘t’ test, *p<0.03, **p<0.005, respectively). (e) Western blot detecting MMP12 levels in control and MMP12 siRNA treated HaCaT or mouse keratinocytes (KRTs) used for the scratch assay is presented. Scratch assay using control, Agrin depleted and Agrin depleted HaCaT/mouse KRTs pre-treated with 10 μg/ml sAgrin for 18 h. Representative bright-field images at the indicated time-points post-wounding are shown. Results quantified as wound area left non-migrated for each group presented as mean+/−s.d. (n=3, Students ‘t’ tests, **p<0.005, ***p=0.0001, respectively). Scale bar: 50 μm. (f) Western blot showing the MMP12 knockdown in the respective mouse KRTs and DFs. GAPDH served as loading controls. Representative bright-field images of control and MMP12 depleted primary mouse keratinocytes and dermal fibroblasts migrating either alone or in the presence of 10 μg/ml sAgrin embedded in soft matrix (0.8 KPa) at indicated days. Scale bar: 100 μm. The relative migration area was quantified by ImageJ (n=4 wounds per group, Multiple ‘t’ tests, ***p=0.0001 and ****p=0.00006, respectively) (g) Mouse skin explants placed on soft collagen substrates alone or having 20 μg/ml sAgrin were either treated with solvent (DMSO) or MMP408 (5 nM) for 5 days. Representative bright-field images showing keratinocyte outgrowth from the explants on day 5 are shown. Scale bar: 50 μm. The outgrowth area was quantified using ImageJ and represented as mean+/−s.d. (n=4 explants per group, Students ‘t’ test, **p=0.002). (h) HaCaT cells were treated with control and MMP12 siRNAs. Three days later, one batch of MMP12 depleted cells was treated with 10 μg/ml sAgrin for 18 h. Subsequently, confluent cells were scratched and at the indicated time-points were fixed and stained for pMLC, Actin, and DAPI. Confocal images of migrating cells are shown. Scale bar: 10 μm. White arrows point towards actomyosin cables. (i) HEK cells treated the same as in (h) were allowed to migrate for the indicated time-points. Cell lysates from migrating cells were tested by Western blot analysis for the indicated proteins. GAPDH served as a loading control. (j) Heat-map showing traction stress displayed by control and MMP12 depleted HaCaTs in the absence or presence of 10 μg/ml sAgrin for 18-24 h. Scale bar: 100 μm. Leading edge stress in Pascals (Pa) are presented graphically (n=6-10 images from three experiments, Students ‘t’ test, *p=0.007, ns p=0.06, upon removal of outliers *-siControl vs siMMP12 *p=0.01, siControl vs siMMP12+sAgrin **p=0.001, respectively Students ‘t’ test, *p=0.007) (k) MMP12 depleted HaCaT cells were labeled with sAgrin or FN conjugated magnetic beads for 30 min at 4° C. before wound-scratch. The migrating cells at 37° C. were either left alone or placed under a permanent magnetic force for 30 min after wounding, fixed, and stained for pMLC, Actin, and DAPI, respectively. Representative confocal images are shown. Scale bar: 10 μm. White circles represent the beads. Mean pMLC intensity+/−s.d. was quantified in a 200μ² area around each bead from three independent experiments (n=10 cells per group, Students ‘t’ test, ns). The results show that MMP12 is a downstream effector for Agrin's mechanoperception.

FIG. 13 shows effects of depletion of MMP1 and MMP10 on wound healing and cellular mechanotension. (a) RT-PCR analysis confirming the suppression of MMP1 and MMP10 in HaCaT cells. Control, MMP1 and 10 depleted HaCaTs and those pre-treated with 10 μg/ml sAgrin for 18 h were subjected to scratch wound assays. Representative bright-field images showing relative migration are presented at the indicated time-points. The mean non-migrated area+/−s.d. was quantified using ImageJ (n=3, Students ‘t’ test, p values indicated in the figure). Scale bar: 50 μm. (b) Comparison of migration velocities of indicated knockdown cells either alone or treated with 10 μg/ml sAgrin at 15 h post-wounding (n=4, Students ‘t’ tests, ****p=0.00001, ***p=0.0001, **p=0.001, respectively). (c) RT-PCR analysis showing mRNA expression of MMP1 and 10 at indicated time-points post-wounding in HaCaT cells. (d) Control, MMP1 and MMP10 depleted HaCaT cells were scratched and allowed to migrate for 4 h. The cells were subsequently fixed and immunostained for pMLC and F-actin. Representative confocal images are shown. Nuclei were counter stained with DAPI. White arrows indicate acto-myosin cables at the leading edge. Scale bar: 10 μm. (e) Control, MMP1 and MMP10 depleted HaCaT cells were plated on large crossbow patterns for 6 h. The cells were subsequently fixed and stained for pMLC and F-actin, with DAPI marking the cell nuclei. White arrows refer to pMLC enriched transverse arcs. The stress curvature associated are enriched with pMLC was quantified as mean+/−s.d. from three independent experiments (n=6 cells per group, Students ‘t’ test, p=0.5 and p=0.16, respectively). The results show that depletion of MMP1 and MMP10 impairs wound healing without affecting cellular mechanotension.

FIG. 14 shows effects of Agrin on MMP12 expression, cellular mechanics and collective fluidic migration. (a) Cell lysates of control and Agrin siRNA treated cells were analyzed by Western blot for MMP12 and Agrin expression. Actin and GAPDH served as loading controls, respectively. (b) Gelatin and casein zymography detecting the catalytic activity of MMP12 in control and Agrin depleted HaCaT cell supernatants. The cell supernatants of Control, Agrin siRNA treated alone, or those treated with an increasing dose of sAgrin for 24 h were analyzed for MMP12 catalytic activity by gelatin (left) or casein (right) in-gel digestion. The appearance of MMP12 bands (˜54 KDa) are a result of gelatin digestion by MMP12. Representative image from one of three independent experiments is shown. The relative MMP12 band intensity was quantified using ImageJ (n=3, Students ‘t’ test, *p=0.02 and 0.03, respectively, Multiple t tests, p values presented in figure). (c) RT-PCR analysis detecting MMP12 mRNA in migrating HaCaTs at 0 h and 24 h post-wound injury. Data presented as mean+/−s.d. Indicated cells were wounded and allowed to migrate. At the indicated time-points, cell lysates from the migrated cells were collected and analyzed by Western blot for MMP12. GAPDH served as loading controls. (d) Confluent HaCaT cells were either left untreated or pre-treated with sAgrin (10 μg/ml) for 18 h, before scratching them and allowing migration for 6 h. At the indicated time-points, cells were fixed and immunostained for MMP12 and Agrin, respectively. Representative confocal images are shown. White arrows indicate leader cells at the migrating edges. Scale bar: 10 μm. (e) Representative confocal microscopy images of control and Agrin depleted mice skin sections showing MMP12 expression at the wound edges. The White dashed line indicates the migrating epithelial tongue. Nuclei are counterstained with DAPI. Scale bar: 50 μm. (f) Western blot analysis showing MMP12 levels on control and Agrin depleted mouse skin tissues at day 4 under splinted conditions. GAPDH was used as a loading control. (g) Control and Agrin depleted HaCaT cells were cultured on stiff substrates. One batch of Agrin depleted cells were cultured on stiff substrates that contained 20 μg/ml sAgrin. Western blot confirming the loss of MMP12 expression in HaCaT cells. GAPDH was used as a loading control. Upon confluency, the cells were scratched and allowed to migrate for 4 h. The cells were then fixed and immunostained for K17. The dashed line denotes the migrating front. The K17 intensity in the leader cells was quantified as mean+/−s.d. from two independent experiments (n=20-30 cells, Students ‘t’ test, *p=0.009 and 0.04, respectively). (h-i) HaCaT cells were either untreated or pre-treated with 10 μg/ml sAgrin for 18 h. Subsequently, the cells were pre-treated with solvent (DMSO) or 5 nM MMP408 inhibitor for 2 h, before wounding them and allowing them to migrate for an additional 4 h in the presence of solvent or inhibitor. Confocal images showing K17 and nuclei stained with DAPI (g) are represented. The White dashed line presents the migrating front. Scale bar: 10 μm. Bright-field microscope images at indicated times show the migrating cells (h). The uncovered wound area was quantified using ImageJ from three independent experiments was presented as mean+/−s.d. (Students ‘t’ test, *p<0.05, **p<0.005, respectively). Scale bar: 50 μm. (j) Confocal images revealing the K17 immunostained outgrowths from mouse skin explants cultured on soft ECM alone or those supplemented with sAgrin in the presence of DMSO or 5 nM MMP408 for 5 days (n=2 independent experiments, with 3 skin explants per group). The white dashed line represents the outgrowth area. Scale bar: 50 μm. (k) Control and MMP12 depleted HaCaT cells were plated on large FN-coated micropatterns for 4-6 h. Besides, a batch of MMP12 depleted cells were plated on FN patterns containing 20 μg/ml sAgrin for 4-6 h. The cells were subsequently fixed and stained for pMLC and F-actin, with DAPI marking the cell nuclei. White arrows refer to pMLC enriched transverse arcs. The stress curvature associated are enriched with pMLC was quantified as mean+/−s.d. from three independent experiments (n=15-20 cells per group, Students ‘t’ test, **p=0.001-0.004). Scale bar: 10 μm. (1) AFM computation of cell stiffness in control, MMP12 depleted and MMP12 knockdown primary mouse keratinocytes treated with sAgrin (n=10-15 cells analyzed from three replicates, Students ‘t’ test, *p=0.01) (m) PIV analysis for fluidic velocity during collective migration of control, MMP12 knockdown cells and those treated with sAgrin. Results are shown from one out of three independent experiments. Green arrows represent the velocity vectors. Scale bar: 100 μm. The migration velocities are quantified as mean+/−s.d. from four independent experiments (Students ‘t’ test, ***p<0.0001). Agrin-regulated MMP12 upgrades cellular mechanics and collective fluidic migration.

FIG. 15 shows knockdown of MMP12 in the mouse skin. (a) Mice were treated with control or MMP12 specific siRNAs. Three days later skin tissues were either collected in the presence or absence of 200 μg sAgrin, and Western blotted for MMP12 levels. GAPDH served as loading controls. (b) Confocal imaging of skin tissues of mice treated same as in (a) at day 10 post-wound injury. White dashed line represents the epidermal and dermal boundaries. Scale bar: 10 μm.

FIG. 16 shows results of assays investigating the effect of MMP12 depletion in Agrin induced wound healing. (a) Control siRNA or mouse specific MMP12 siRNA injected at the prospective wound sites on mouse skin. Three days later, the sites were wounded and an Aquaphore based ointment containing either control or MMP12 siRNA in the presence or absence of 200 μg sAgrin were applied on the splinted wounds every alternate day. Photographs of mouse skin wounds at indicated days are presented. Scale bar: 1 mm. The wound diameter is quantified at indicated days (n=3-4 mice per group, ANOVA, *p=0.007, *p=0.01, ***p=0.0001, respectively). (b) Representative Haematoxylin and Eosin stained sections of mouse skin receiving the combinations of siRNA treatments as in panel (a) at day 10 post-wounding. Region encoded by black arrows represents the unhealed area. Scale bar: 100 μm. (c) Representative confocal images showing the pMLC staining in control and MMP12 depleted mouse skin in the absence or presence of sAgrin treatments. Scale bar: 10 μm. The pMLC intensity was quantified using the Zeiss Fluoview (n=3 independent mouse sections per group, Students ‘t’ test, *p=0.01, **p=0.005 and 0.002, respectively). (d) Picrosirius red staining images of indicated mouse skin sections at day 10 post-wounding. Representative bright-field and polarized views are presented. Scale bar: 100 μm. WB denotes wound bed. The distribution of individual types of collagen fibers were done using ImageJ and presented graphically (n=3 sections per group, Students ‘t’ test, p values presented in the table). (e) Detection of angiogenesis by CD-31 immunohistochemistry analysis in control and MMP12 deficient mouse skin wound beds in the presence or absence of sAgrin treatment at 10 days post-injury. The number of blood vessels and their diameter are represented graphically as mean+/−s.d. (n=3 mice per group, 30-60 blood vessels from each image from three mice per group were analyzed, ANOVA, *p=0.02, Multiple t tests, *p=0.02, p=0.01, p=0.008, **p=0.0009, respectively). The results show that MMP12 is required for sAgrin induced wound healing in vivo.

FIG. 17 shows results of assays investigating whether sAgrin can be used as a bio-additive skin wound-healing material. (a) The protein sequence of the C-terminal Agrin fragment used as bio-additive. Gel-filtration profile of purified sAgrin. The recombinant sAgrin were tested in a Coomassie stained gel and by Western blot using an Agrin antibody. (b) BrDu proliferation assay of mouse keratinocytes cells treated with increasing concentrations of sAgrin for the indicated days (n=3 experiments, Students ‘t’ test, **p=0.001 and *p=0.01, respectively). (c) HaCaT/mouse keratinocytes cells treated with increasing doses of sAgrin were subjected to scratch wound assay. Representative bright-field images are shown at indicated times post-wounding. Scale bar: 20 μm. The resulting uncovered wound area presented as mean+/−s.d. was quantified using ImageJ (n=3, Students ‘t’ test, *p<0.01). (d) Untreated or sAgrin (10 μg/ml) HaCaT cells were scratched and allowed to migrate for 4 h. Subsequently, the cells were fixed and immunostained for K17 and nuclei was stained with DAPI. Representative confocal images are presented. White arrows point towards the direction of migration. The white dashed line represents the migrating front. Scale bar: 10 μm. (e) Confocal images showing K17 staining from mouse skin explants cultured for 5 days on soft substrates alone or those supplemented with sAgrin (20 μg/ml). Ex refers to explant and the dashed line surrounding Ex represents the border of explant. The region between the dashed lines presents the outgrowth areas, respectively. Scale bar: 50 μm. The experiment was done three times with three explants per group. (f) Agrin depleted HaCaTs were treated with sAgrin or FN coated beads for 30 min before wounding them. The cells were either left alone or placed under magnetic force and allowed to migrate for 30 min before they were fixed and immunostained for MMP12 and pMLC. Nuclei were counterstained with DAPI. Representative confocal images are shown (n=2 experimental replicates). Scale bar: 10 μm. (g) Vaseline based ointments containing either 200 μg BSA (control) or increasing amounts of sAgrin were applied topically to punch-wounds in mouse skin every 2-3 days. The representative photographs of the treated mice wound regions are shown on indicated days. Scale bar: 1 mm. The relative diameter of wounds presented as mean+/−s.d. was measured on indicated days and represented graphically from one experiment (n=3 mice per group, One-way ANOVA, *p<0.04, **p<0.004, respectively). (h) Ki67 stained sections of BSA or sAgrin treated mouse skin at day 7 post-injury. The degree of proliferating cells were quantified using ImageJ (n=3 sections analyzed from two independent mice wounds, Students ‘t’ tests, *p=0.01). (i) Picrosirius red stained images of BSA and sAgrin treated mouse skin sections (non-splinted model) at day 7 post-wounding. Representative bright-field and polarized views are presented. Scale bar: 100 μm. White arrows represent the unhealed area. The quantification of individual types of collagen fibers were done using ImageJ (n=3 sections analyzed per group, Students ‘t’ test, *p=0.01 and 0.05, respectively). The results show that sAgrin accelerated in vitro and in vivo wound healing which may have clinical relevance as a wound healing biomaterial.

FIG. 18 shows results of assays investigating the effect of Agrin-based topical biomaterial on skin wound healing. (a-b) BSA or Agrin (200 μg) incorporated Pluronic-F-127 based ointments applied to punch-wounds in mice every two days. Wounds receiving the indicated ointments were left uncovered in panel (a-non-splinted). For panel b, 200 μg of rat-tail collagen was used as an additional control along-with sAgrin and BSA. The wounds were tethered with a 10 mm transparent nylon splint and the ointment applied were covered by Tegaderm (splinted). Representative photo showing the punch wound area in mice receiving the above treatments, respectively. Scale bar: 1 mm. Mean wound area+/−s.d. plotted graphically over time since wound generation (n=4 mice per group, ANOVA, p<0.0001, p=0.001, p=0.0001, respectively). (c) Hematoxylin and Eosin stained bright-field images of control and Agrin treated wound edges at day 2 post-injury. Asterisk (*) denotes the migrating keratinocytes forming epithelial tongue at the wound edge. The two-directional black arrow (non-splinted) shows the length of the migrating keratinocytes at day 2 post-wounding. The black arrows (splinted) present the area left unhealed at day 10 post-wounding. Scale bar: 100 μm. (d-) Immunofluorescence confocal images showing the expression of K17 occupancy in the mouse skin wounds receiving BSA, Collagen or sAgrin treatments in non-splinted (at day 2) or splinted conditions (at day 7). Relative K17 intensity is shown graphically (n=3-6 sections analyzed from 3 mice per group, Students ‘t’ test, *p=0.01, **p=0.005, ***p=0.0001, respectively). (e-f) Representative confocal immunofluorescence images showing MMP12 (e) and pMLC (f) within the wound edges and at the wound beds of mice skin receiving BSA, Collagen or Agrin ointments at day 2 (in non-splinted models) or day 7 (splinted models) post-injury. Relative staining intensities are presented as mean+/−s.d. from three to four sections from three mice (Students ‘t’ test, *p=0.02, p=0.04, p=0.03, p=0.01, and ***p=0.001, respectively). Scale bar: 100 μm. The dashed white line denotes the thickness of the migrating epidermis, while the solid white line separates keratinocytes from underlined dermis regions. (g) Picrosirius red stained images of BSA, Collagen and sAgrin treated mouse skin sections (splint model) at day 10 post-wounding. Representative bright-field and polarized views are presented. Scale bar: 100 μm. WB denotes wound bed. White arrows represent the unhealed area. The quantification of individual types of collagen fibers were done using ImageJ (n=3 sections analyzed, three mice per group, Students ‘t’ test, p values shown in the figure). (h) CD-31 immunohistochemistry analysis in BSA, Collagen and Agrin treated mice skin wound beds at indicated days post-injury in non-splinted and splinted conditions. Arrows denote blood vessels. The number of blood vessels and their diameter are represented graphically as mean+/−s.d. (n=5-10 sections analyzed from three mice per group, Students ‘t’ test, *p=0.01, *p=0.04, ANOVA *p=0.02, and ***p=0.0001, respectively). The results show that Agrin-based topical biomaterial accelerated skin wound healing.

FIG. 19 shows profile of pro-inflammatory proteins induced by sAgrin during early phases of wound healing. (a) Heatmap showing the mRNA expression(s) of commonly induced cytokines and chemokines at 4 h and 48 h post-wounding in mouse skin that received BSA, Collagen or sAgrin treatments. The photographs of a representative wound for each condition is shown above. The respective p values for the selected group of significant genes are represent in tabular form (n=3 mice per group, Multiple t tests). (b) Western blot analysis in the mouse skin treated as in (a) showing the protein expression of selected proteins. GAPDH served as a loading control (n=2 independent mice wounds were analyzed for each group).

FIG. 20 shows results of assays investigating the effect of Agrin on angiogenesis. (a) Western blot analysis in control and Agrin siRNA treated BJ-GFP cells and HDMEC labelled with Cell Tracker blue for 18 h. GAPDH was used as a loading control. (b) Cells treated as in panel (a) were co-cultured to form spheroids for 24 h. Bright-field images of sprouting is shown for each indicated condition. The respective fibroblasts and HDMEC spheroids are depicted by green and blue fluorescence, respectively. Scale bar: 100 μm. Sprouting was quantified using ImageJ (n=3 spheroids per condition, Students ‘t’ test, *p=0.05 and p=0.02, respectively). The results show that Agrin mediates angiogenesis by fostering interactions between dermal fibroblasts and endothelial cells.

FIG. 21 shows a working model explaining that wound injury triggered the Agrin microenvironment favors a productive healing program. Briefly, Agrin sensitizes the wounded cells towards ECM rigidity, force recognition and geometric constraints accounting for improved traction stress, elasticity and cytoskeletal tension to promote enhanced fluid-like dynamic collective migration over the wounded sites. Further, Agrin deploys MMP12 as its downstream effector to upgrade the mechanoperception of keratinocytes and actomyosin integrity during keratinocyte migration. The cumulative outcome of an Agrin-driven mechanically competent wound healing micro-environment yields higher collective migration and wound closure via facilitating re-epithelization, optimal ECM deposition and angiogenesis.

FIG. 22 shows experimental set up for imaging traction force in collectively migrating cells post-wounding. (a) Image showing the 3D-printed mould and casted hydrogel block. (b) The image illustrates the intended use of polyacrylamide block on glass bottom dishes to create artificial wounds.

### DETAILED DESCRIPTION OF THE PRESENT INVENTION

The stages of wound healing proceed in an organized way and follow four processes: hemostasis, inflammation, proliferation and maturation. Hemostasis is the process of the wound being closed by clotting. Inflammation is the second stage of wound healing and it controls bleeding and prevents infection. Proliferation is when the wound is rebuilt with new tissue made up of collagen and extracellular matrix (ECM). The maturation phase is when collagen is remodeled from type III to type I and the wound fully closes. The proliferative phase comprises angiogenesis, fibroplasia and granulation tissue formation, collagen deposition, re-epithelialization, and contraction, and is the key phase of wound healing. Re-epithelialization is the key step in the proliferative phase. Keratinocytes are the main cells responsible for re-epithelialization. Activated keratinocytes at the wound edge migrate across the wound bed until the epithelial tongues meet at the wound center. From here, keratinocytes differentiate to form a new skin barrier. On epidermal wounding, keratinocytes at the wound edge undergo a transition from a non-motile epithelial state to a mesenchymal-like state, where they lose cell-cell contacts and become motile. Migrating cells reorganize their actin cytoskeleton and secrete proteases to remodel the ECM and enable migration across the wound. Directly behind the migrating cells, keratinocytes rapidly proliferate to provide enough cells to cover the wound. Non-healing wounds are characterized by defective keratinocyte migration, where, as a result, re-epithelialization fails to occur.

It has been shown in the present disclosure that Agrin expression is significantly triggered within the epidermal and dermal layers of skin upon mechanical injury, and that supplementing sAgrin (the C-terminus recombinant protein fragment of Agrin harboring the binding sites to its receptors Lipoprotein related receptor-4 (LRP4) and integrins) significantly rescued the wound healing and the migration of keratinocytes. Thus, in a first aspect, the present invention refers to a pharmaceutical composition comprising an Agrin fragment or derivative thereof, wherein the Agrin fragment or derivative thereof comprises the LG3 domain of Agrin and an eight-amino-acid insert ELANEIPV (SEQ ID NO: 1) at the z-site of the LG3 domain. The Agrin fragment or derivative thereof as defined above contains the binding site to Lipoprotein related receptor-4 (LRP4) and integrins.

In some examples, the Agrin fragment or derivative thereof further comprises the LG2 domain of Agrin.

Agrin is a large heparan proteoglycan with a molecular weight of 400-600 kDa. The protein core of Agrin consists of about 2000 amino acids with a mass of about 225 kDa. Agrin is a multidomain protein composed of 9 K (kunitz-type) domains, 2 LE (laminin-EGF-like) domains, one SEA (sperm protein, enterokinase and agrin) domain, 4 EG (epidermal growth factor-like) domains and 3 LG (laminin globular) domains. Agrin exists in several splice variants and can be expressed as a secreted protein, containing the N-terminal NtA (N-terminal Agrin) domain, which is the most abundant form of Agrin. The C-terminal, 75 kDa moiety of Agrin starts with the first EG domain. Several binding sites for interaction partners of Agrin, including α-dystroglycan, heparin, some integrins and LRP4 are mapped to the C-terminal region. In the C-terminal part of human Agrin, there are two alternative splice sites y and z. The y-site is located within the LG2 domain, and the z-site is located within the LG3 domain. At the y-site, there may be inserts of 0, 4, 17 or 21 (4+17) amino acids; and at the z-site, there may be inserts of 0, 8, 11 or 19 (8+11) amino acids.

Lipoprotein receptor-related protein 4 (LRP-4), also known as low-density lipoprotein receptor-related protein 4, is a protein that in humans is encoded by the LRP4 gene. LRP-4 is a member of the Lipoprotein receptor-related protein family and may be a regulator of Wnt signaling.

Integrins are transmembrane receptors that facilitate cell-cell and cell-ECM adhesion. Upon ligand binding, integrins activate signal transduction pathways that mediate cellular signals such as regulation of the cell cycle, organization of the intracellular cytoskeleton, and movement of new receptors to the cell membrane. The presence of integrins allows rapid and flexible responses to events at the cell surface.

The terms “LG2” and “LG3” as used herein refers to the second and third laminin globular domains of Agrin. LG2 and LG3 shall encompass all possible different splice variations of these domains. In one example, the LG2 domain of Agrin without any insert at the y-site has the sequence PFLADFNGFSHLELRGLHTFARDLGEKMALEVVFLARGPSGLLLYNGQKTDGKGDF VSLALRDRRLEFRYDLGKGAAVIRSREPVTLGAWTRVSLERNGRKGALRVGDGPRV LGESPVPHTVLNLKEPLYVGGAPDFSKLARAAAVSSGFDGAIQLVSLGGRQLLTPEH VLRQVDVTSFAGHPC (SEQ ID NO: 2). In one example, the LG2 domain of Agrin having an insert of 4 amino-acids at the y-site has the sequence of PFLADFNGFSHLELRGLHTFARDLGEKMALEVVFLARGPSGLLLYNGQKTDGKGDF VSLALRDRRLEFRYDLGKGAAVIRSREPVTLGAWTRVSLERNGRKGALRVGDGPRV LGESPVPKSRKHTVLNLKEPLYVGGAPDFSKLARAAAVSSGFDGAIQLVSLGGRQLL TPEHVLRQVDVTSFAGHPC (SEQ ID NO: 3), with the sequence of the insert being KSRK (SEQ ID NO: 4). In the above mentioned LG2 domain, the y-site starts at P (proline) 119. In one example, the Agrin fragment or derivative as described herein comprises the LG2 domain of Agrin without any insert at the y-site. In one example, the LG3 domain of Agrin without any insert at the z-site has the sequence EYLNAVTESEKALQSNHFELSLRTEATQGLVLWSGKATERADYVALAIVDGHLQLS YNLGSQPVVLRSTVPVNTNRWLRVVAHREQREGSLQVGNEAPVTGSSPLGATQLDT DGALWLGGLPELPVGPALPKAYGTGFVGCLRDVVVGRHPLHLLEDAVTKPELRPC (SEQ ID NO: 5). In another example, the LG3 domain of Agrin having an insert of 8 amino-acids at the z-site has the sequence of EYLNAVTESELANEIPVEKALQSNHFELSLRTEATQGLVLWSGKATERADYVALAIV DGHLQLSYNLGSQPVVLRSTVPVNTNRWLRVVAHREQREGSLQVGNEAPVTGSSPL GATQLDTDGALWLGGLPELPVGPALPKAYGTGFVGCLRDVVVGRHPLHLLEDAVT KPELRPC (SEQ ID NO: 6), with the sequence of the insert being ELANEIPV (SEQ ID NO: 1). In the above mentioned LG3 domain, the z-site is between S (serine) 9 and E (glutamic acid) 10. In some examples, the Agrin fragment or derivative thereof comprising the LG3 domain of Agrin and an eight-amino-acid insert at the z-site of the LG3 domain has the following sequence:

(SEQ ID NO: 7)
DTLAFDGRTFVEYLNAVTESELANEIPVEKALQSNHFELSLRTEATQGL
VLWSGKATERADYVALAIVDGHLQLSYNLGSQPVVLRSTVPVNTNRWLR
VVAHREQREGSLQVGNEAPVTGSSPLGATQLDTDGALWLGGLPELPVGP
ALPKAYGTGFVGCLRDVVVGRHPLHLLEDAVTKPELRPCPTP.

In some examples, the Agrin fragment or derivative thereof comprising the LG2 and LG3 domains of Agrin and an eight-amino-acid insert at the z-site of the LG3 domain has the following sequence:

(SEQ ID NO: 8)
LGREGTFCQTASGQDGSGPFLADFNGFSHLELRGLHTFARDLGEKMALE
VVFLARGPSGLLLYNGQKTDGKGDFVSLALRDRRLEFRYDLGKGAAVIR
SREPVTLGAWTRVSLERNGRKGALRVGDGPRVLGESPVPHTVLNLKEPL
YVGGAPDFSKLARAAAVSSGFDGAIQLVSLGGRQLLTPEHVLRQVDVTS
FAGHPCTRASGHPCLNGASCVPREAAYVCLCPGGFSGPHCEKGLVEKSA
GDVDTLAFDGRTFVEYLNAVTESELANEIPVEKALQSNHFELSLRTEAT
QGLVLWSGKATERADYVALAIVDGHLQLSYNLGSQPVVLRSTVPVNTNR
WLRVVAHREQREGSLQVGNEAPVTGSSPLGATQLDTDGALWLGGLPELP
VGPALPKAYGTGFVGCLRDVVVGRHPLHLLEDAVTKPELRPCPTP.

As additional variations of sequence which do not affect the biological activity are possible, the invention shall not be limited to the indicated sequences of the different splice variants of the domains LG2 and LG3. In some examples, the Agrin fragment or derivative thereof comprises sequences which have at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99% sequence identity as the exemplary LG2 and/or LG3 domain sequences as provided herein.

The term “derivative” as used herein refers to a polypeptide that has been derived from the basic sequence by modification, including amino acid deletions or additions to polypeptides or variants and modification to side chains, where the derivative retains the activity of the basic protein. The resulting derivative will retain at least about at least about 60%, or at least about 65%, or at least about 70%, or at least about 75%, or at least about 80%, or at least about 85%, or at least about 90%, or at least about 95%, or at least about 96%, or at least about 97%, or at least about 98%, or at least about 99% homology with the basic sequence of the original polypeptide. The derivative will also exhibit a qualitatively similar effect to the unmodified polypeptide.

In some examples, the Agrin fragment or derivative thereof additionally includes at least one further domain naturally occurring in Agrin, including but not limited to, the LG1 and EGF-like domains EG1-4. Exemplary sequences of LG1 and EG1-4 domains are as follows:

LG1 domain:
(SEQ ID NO: 9)
APVPAFEGRSFLAFPTLRAYHTLRLALEFRALEPQGLLLYNGNARGKDF
LALALLDGRVQLRFDTGSGPAVLTSAVPVEPGQWHRLELSRHWRRGTLS
VDGETPVLGESPSGTDGLNLDTDLFVGGVPEDQAAVALERTFVGAGLRG
CIRLLDVNNQRLELGIGPGAATRGSGVGEC.
EG1 domain:
(SEQ ID NO: 10)
PPKPCDSQPCFHGGTCQDWALGGGFTCSCPAGRGGAVCE.
EG2 domain:
(SEQ ID NO: 11)
GDHPCLPNPCHGGAPCQNLEAGRFHCQCPPGRVGPTCA.
EG3 domain:
(SEQ ID NO: 12)
EKSPCQPNPCHGAAPCRVLPEGGAQCECPLGREGTFCQ.
EG4 domain:
(SEQ ID NO: 13)
AGHPCTRASGHPCLNGASCVPREAAYVCLCPGGFSGPHCE.

Agrin fragments or derivatives thereof can be obtained by usual recombinant engineering which is well known in the art and exemplified in the working examples of the present disclosure. In summary, nucleic acid molecules encoding for the Agrin fragment or derivative thereof is expressed in suitable expression systems and the resulting protein is subsequently purified. Thus, in some examples, there are provided a nucleic acid molecule encoding for the Agrin fragment or derivative thereof as disclosed herein. In one example, the nucleic acid molecule encoding for the Agrin fragment or derivative thereof comprising the LG3 domain and the 8 amino acid insert at the z-site has the following sequence: CATATGGACACCCTGGCGTTCGATGGTCGTACCTTTGTTGAGTACCTGAACGCGG TGACCGAGAGCGAACTGGCGAACGAGATCCCGGTTGAAAAGGCGCTGCAGAGC AACCACTTCGAGCTGAGCCTGCGTACCGAAGCGACCCAAGGTCTGGTGCTGTGG AGCGGCAAAGCGACCGAACGTGCGGACTACGTTGCGCTGGCGATTGTGGATGGT CACCTGCAGCTGAGCTATAACCTGGGCAGCCAACCGGTGGTTCTGCGTAGCACC GTTCCGGTGAACACCAACCGTTGGCTGCGTGTGGTTGCGCACCGTGAGCAGCGTG AAGGTAGCCTGCAAGTTGGCAACGAAGCGCCGGTGACCGGTAGCAGCCCGCTGG GTGCGACCCAGCTGGACACCGATGGTGCGCTGTGGCTGGGTGGCCTGCCGGAAC TGCCGGTTGGTCCGGCGCTGCCGAAGGCGTATGGTACCGGCTTTGTGGGTTGCCT GCGTGACGTGGTTGTTGGTCGTCACCCGCTGCACCTGCTGGAGGATGCGGTTACC AAACCGGAACTGCGTCCGTGCCCGACCCCGTAAGGATCC (SEQ ID NO: 14). In one example, the nucleic acid molecule encoding for the Agrin fragment or derivative thereof comprising the LG2 domain, the LG3 domain and the 8 amino acid insert at the z-site has the following sequence:

(SEQ ID NO: 15)
CATATGCTGGGTCGTGAGGGCACCTTCTGCCAAACCGCGAGCGGTCAAG
ATGGTAGCGGTCCGTTTCTGGCGGATTTCAACGGTTTTAGCCACCTGGA
ACTGCGTGGCCTGCACACCTTCGCGCGTGACCTGGGCGAGAAGATGGCG
CTGGAAGTGGTTTTTCTGGCGCGTGGTCCGAGCGGTCTGCTGCTGTACA
ACGGCCAGAAGACCGACGGTAAAGGCGATTTCGTTAGCCTGGCGCTGCG
TGACCGTCGTCTGGAGTTTCGTTATGATCTGGGTAAAGGTGCTGCGGTT
ATTCGTAGCCGTGAGCCGGTGACCCTGGGTGCGTGGACCCGTGTGAGCC
TGGAACGTAACGGTCGTAAGGGTGCGCTGCGTGTTGGTGATGGTCCGCG
TGTGCTGGGCGAGAGCCCGGTTCCGCACACCGTGCTGAACCTGAAGGAA
CCGCTGTACGTTGGTGGCGCGCCGGACTTCAGCAAGCTGGCGCGTGCGG
CGGCGGTTAGCAGCGGTTTTGATGGCGCGATTCAGCTGGTTAGCCTGGG
TGGCCGTCAACTGCTGACCCCGGAGCACGTGCTGCGTCAAGTTGACGTT
ACCAGCTTTGCGGGTCACCCGTGCACCCGTGCGAGCGGTCATCCGTGCC
TGAACGGTGCGAGCTGCGTTCCGCGTGAAGCGGCGTACGTGTGCCTGTG
CCCGGGTGGCTTTAGCGGTCCGCACTGCGAGAAGGGTCTGGTGGAGAAG
AGCGCGGGTGACGTGGATACCCTGGCGTTCGATGGCCGTACCTTTGTTG
AGTATCTGAACGCGGTGACCGAGAGCGAACTGGCGAACGAGATCCCGGT
TGAAAAGGCGCTGCAGAGCAACCACTTCGAGCTGAGCCTGCGTACCGAA
GCGACCCAAGGTCTGGTGCTGTGGAGCGGCAAAGCGACCGAACGTGCGG
ACTACGTTGCGCTGGCGATTGTGGATGGTCACCTGCAGCTGAGCTATAA
CCTGGGCAGCCAACCGGTGGTTCTGCGTAGCACCGTTCCGGTGAACACC
AACCGTTGGCTGCGTGTGGTTGCGCACCGTGAGCAGCGTGAAGGTAGCC
TGCAAGTTGGCAACGAAGCGCCGGTGACCGGTAGCAGCCCGCTGGGTGC
GACCCAGCTGGACACCGATGGTGCGCTGTGGCTGGGTGGCCTGCCGGAA
CTGCCGGTTGGTCCGGCGCTGCCGAAGGCGTATGGTACCGGCTTTGTGG
GTTGCCTGCGTGACGTGGTTGTTGGTCGTCACCCGCTGCACCTGCTGGA
GGATGCGGTTACCAAACCGGAACTGCGTCCGTGCCCGACCCCGTAAGGA
TCC.

In some examples, there are provided a vector comprising the nucleic acid molecule encoding for the Agrin fragment or derivative thereof as disclosed herein. The term “vector” as used herein includes vectors which can be used to express DNA sequences contained therein, where such DNA sequences are operably linked to other sequences capable of effecting their expression (e.g., promotor/operator sequences). In general, expression vectors of utility in recombinant DNA techniques are often in the form of “plasmids” which refer to circular double stranded DNA loops which in their vector form, are not bound to the chromosome. Various expression vectors known in the art can be used to obtain a vector comprising the nucleic acid molecule encoding for the Agrin fragment or derivative thereof as disclosed herein, including but are not limited to, bacterial expression vectors such as pET28, pUC19, pBR327, pBR322, pET3a, pEXP4-DEST, pSP72, pET SUMO, pBAD TOPO, pGEX-4T2, pQE-30 and pACYC177 vectors, and mammalian expression vectors such as pACT, pBIND, pG5luc, pTNT, pTarget, pReg neo, pCat3-Basic, pSI, pcDBA and pCMV vectors. In one example, the expression vector is a pET28 vector. In one specific example, the expression vector is a pET28 vector containing His-tag.

The present disclosure also provides host cells comprising the vector comprising the nucleic acid molecule encoding for the Agrin fragment or derivative thereof as disclosed herein. Several prokaryotic and eukaryotic expression systems are suitable for the production of the Agrin fragment or derivative thereof as disclosed herein. Prokaryotic expression systems include, but are not limited to, expression in Escherichia coli (E. coli). Eukaryotic expression systems include expression in mouse myeloma cells, baculovirus-mediated expression in insect cells, as well as expression in human embryonic kidney (HEK) cells, transient expression in Chinese hamster ovary (CHO) cells and stable expression in Pichia pastoris. These systems have the advantage that they can easily be adapted to serum-free conditions to reduce the amount of contaminating proteins in the supernatant and can be adapted for large scale production. In addition, a variety of cell lines may be used, including HEK293T and HEK293-cells, COS cells, CHO cells, HeLa cells, H9 cells, Jurkat cells, NIH3T3 cells, C127 cells, CV1 cells, CAP cells or SF cells. Thus, in one example, the present disclosure provides a host cell comprising the vector comprising the nucleic acid molecule encoding for the Agrin fragment or derivative thereof as disclosed herein. In one specific example, the host cell is an E. coli cell. The present disclosure also provides a method of producing the Agrin fragment or derivative thereof as disclosed herein, the method comprising culturing the host cells as disclosed herein to express the Agrin fragment or derivative thereof, and harvesting the Agrin fragment or derivative thereof produced. In some examples, the method further comprises purification of the Agrin fragment or derivative thereof obtained.

For the purification of the Agrin fragment or derivative thereof obtained, standard protein purification technologies can be applied. His-tagged protein can be purified using IMAC, and ion exchange chromatography or affinity purification using a heparin column can be used as well. Purification via an antibody raised against the C-terminal part of Agrin can also be used. The eluted protein can then further be purified using, for example, a hydroxyapatite column or by gel filtration.

Agrin fragment or derivative thereof as disclosed in the present application can be in either the secreted or transmembraneous form. In some examples, Agrin fragment or derivative thereof is in the secreted form. In some examples, the Agrin fragment or derivative thereof is soluble.

The Agrin fragment or derivative thereof as disclosed in the present application can be of any origin. In some examples, the Agrin fragment or derivative thereof is derived from human, non-human primates, mouse, rat, hamster, rabbit, goat or other mammalian species. In some specific examples, the Agrin fragment or derivative thereof is derived from human or mouse.

In some examples, the pharmaceutical compositions disclosed herein comprise about 0.01% to about 25%, or about 0.01% to about 10%, or about 0.03% to about 1%, or about 0.03% to about 5%, or about 1% to about 10% w/v, or about 6%, 8%, 10%, 15% or 20% w/v of Agrin fragment or derivative thereof.

In some examples, the pharmaceutical compositions disclosed herein further comprise other active agents acting synergistically on the wound for the promotion of wound healing or wound closure or the treatment of non-healing wounds.

In some examples, the pharmaceutical composition further comprises a pharmaceutically acceptable carrier and/or other pharmaceutically acceptable inert agents.

The term “pharmaceutically acceptable” as used herein refers to ingredients, agents, or compositions that are suitable for pharmaceutical administration without undue toxicity, incompatibility, instability, irritation, allergic response and the like.

The term “carrier” as used herein refers to diluents, adjuvants, excipients, vehicles, and other inert agents with which the Agrin fragment or derivative thereof is administered.

Examples of pharmaceutically acceptable carriers include but are not limited to sugars, starches, cellulose, excipients, oils, glycols, polyols, esters, agar, and buffering agents. The above are non-limiting examples of carriers. Pharmaceutically acceptable carriers may be easily formulated by those of ordinary skill in the art.

Examples of excipients include but are not limited to, solvents, emollients and/or emulsifiers, oil bases, preservatives, antioxidants, tonicity adjusters, penetration enhancers and solubilizers, chelating agents, buffering agents, surfactants, one or more polymers, and combinations thereof. Suitable solvents for an aqueous or hydrophilic topical formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for a hydrophobic topical formulation include mineral oils, vegetable oils, and silicone oils. If desired, the pharmaceutical composition as described herein may be dissolved or dispersed in a hydrophobic oil phase, and the oil phase may then be emulsified in an aqueous phase comprising water, alone or in combination with lower alcohols, glycerin, and/or glycols. Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids. Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil. Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl 5 myristate, oleyl stearate, and oleyl oleate. Specific examples of alkyl esters of diacids include diisopropyl adipate, diisohexyl adipate, bis(hexyldecyl) adipate, and diisopropyl sebacate. Other suitable classes of emollients or emulsifiers which may be used in topical formulations include fatty acids, fatty alcohols, fatty alcohol ethers, ethoxylated fatty alcohols, fatty acid esters of ethoxylated fatty alcohols, and waxes. Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol. Specific examples of waxes suitable for use as emollients include lanolin and derivatives thereof, including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes. Polyhydric alcohols and polyether derivatives may be used as solvents and/or surfactants in topical formulations. Suitable polyhydric alcohols and polyethers include propylene glycol, dipropylene glycol, polypropylene glycols 2000 and 4000, poly(oxyethylene co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-15 hexanediol, vicinal glycols having 15 to 18 carbon atoms, and polyoxypropylene derivatives of trimethylolpropane. Polydydric alcohol esters may be used as emulsifiers or emollients. Suitable polydydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters. Suitable emulsifiers for use in topical formulations include anionic, cationic, nonionic, and zwitterionic surfactants. Preferred ionic emulsifiers include phospholipids, such as lecithin and derivatives. Lecithin and other phospholipids may be used to prepare liposomes containing the composition as described herein. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the composition as described herein to keratinocytes by fusing with the cell membrane of the keratinocytes. Sterols including, for example, cholesterol and cholesterol fatty acid esters; amides such as fatty acid amides, ethoxylated fatty acid amides, and fatty acid alkanolamides may also be used as emollients and/or penetration enhancers. Suitable viscosity enhancers or thickeners which may be used to prepare a viscous gel or cream with an aqueous base include sodium polyacrylate, xanthan gum, polyvinyl pyrollidone, acrylic acid polymer, carrageenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Suitable preservatives and/or antioxidants for use in topical formulations include benzalkonium chloride, benzyl alcohol, phenol, urea, parabens, butylated hydroxytoluene (BHT), butylated hydroxyanisole 5 (BHA), Tocopherol, and mixtures thereof. Suitable chelating agents for use in topical formulations include ethylene diamine tetraacetic acid, alkali metal salts thereof, alkaline earth metal salts thereof, ammonium salts thereof, and tetraalkyl ammonium salts thereof. The carrier preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid.

The pharmaceutical compositions disclosed herein may additionally comprise conventional adjuvants such as propionic acid, propylene glycol, conventional buffers, preservatives, hydrophilic emulsifiers, lipophilic emulsifiers, perfumes, emollients, deodorants, humectants and the like. Colorants may also optionally be added in the compositions disclosed herein. Adjuvants which would be harmful to a wound or surrounding skin should be avoided, as well as those adjuvants which may react with and/or adversely reduce the effectiveness of the pharmaceutical composition.

The pharmaceutical compositions disclosed herein may be formulated into a wide variety of articles to be topically applied that include but are not limited to lotions, creams, gels, sticks, sprays, ointments, emulsions, pastes, foams, powders and film-forming products. Such pharmaceutical compositions may be formulated for time-controlled release. If the pharmaceutical composition is formulated into an emulsion, the emulsion may have a continuous aqueous phase and a discontinuous non-aqueous or oil phase (oil-in-water emulsion), or a continuous non-aqueous or oil phase and a discontinuous aqueous phase (water-in-oil emulsion).

In some examples, the pharmaceutical composition as disclosed herein further comprises one or more preservatives. Examples of preservatives include, but are not limited to chelators such as EDTA, diethylene triamine pentaacetic acid (DTPA), and catechins; sodium benzoate; potassium sorbate; and sodium nitrate. The compositions may comprise about 0.01% to about 5%, or about 0.1% to about 3%, or about 0.015% to about 1%, or about 0.015% to about 0.5%, or about 0.01% to about 0.1%, or about 0.0225% to about 0.1% w/v or about 0.015%, 0.225%, or 0.1% w/v of preservatives.

The compositions provided herein may further comprise one or more antimicrobial agents. The antimicrobial agents can act to counter any bacterial protease activity that may hamper the healing environment, which allows a wound to progress towards an optimal healing state. Examples of antimicrobial agents include, but are not limited to, components of aloe vera, ashitaba, bacteriophage, beta-defensin, quaternary ammonium compound, chlorhexidine, copper, dispersin B, essential oil, gentamicin, lactoferrin, lysostaphin, N-halamines, nitric oxide, oleic acid, PLUNC, polyhexanide biguanide (PHMB), bacteriocin, selenium, silver compound, triclosan, zinc, and combinations thereof. Aloe vera contains numerous photochemical compounds including but not limited to tannin, saponin, flavonoids, and fumaric acid. As used herein, the term “PLUNC” refers to the gene or clone encoding the palate, lung, nasal epithelium carcinoma associated protein and to the protein itself. Examples of quaternary ammonium compound include benzethonium chloride and benzalkonium chloride. An example of a beta-defensin is cathelicidin (LL-37). Examples of a silver compound may include colloidal silver, ionic silver, nonionic silver, silver chloride, silver nanoparticles, and silver sulfadiazine. Examples of essential oil include but are not limited to cinnamon oil, clove oil, eucalyptus oil, and tea tree oil. An example of chlorhexidine is chlorhexidine gluconate. The compositions may comprise about 0.01% to about 1%, or about 0.05% to about 1%, or about 0.05% to about 0.5% w/v of antimicrobial agents.

The pharmaceutical compositions disclosed herein may further comprise other agents such as growth factors, cytokines, and proteinase inhibitors. Examples of growth factors include but are not limited to, epidermal growth factor (EGF), transforming growth factor-α (TGF-α), platelet derived growth factor (PDGF), fibroblast growth factors (FGFs) including acidic fibroblast growth factor (α-FGF) and basic fibroblast growth factor (β-FGF), transforming growth factor-β (TGF-β) and insulin like growth factors (IGF-1 and IGF-2), and combination thereof.

In some examples, the pharmaceutical composition as disclosed herein may be infused within, injected into, absorbed by, layered on, encapsulated within, or coated on, a carrier material, such as a bandage, gauze, wound dressing, adhesive bandage, scaffold, or hydrogel. The carrier material may be either bioresorbable, for instance comprising polyglycolic acid, polylactic acid, polydioxanone, polyhydroxybutyrate, polyhydrozyvalerate, polyaminoacids polyorthoesters, polyvinly alcohol, collagen, gelatin, chitosan, oxidized regenerated cellulose, hyaluronic acid, alginate or derivatives thereof, or may be non-bioresorbable, comprising for instance, polyurethane, polyvinyl alcohol, or gauze. Carrier materials are distinct from the carriers and pharmaceutically acceptable carriers used in the pharmaceutical compositions.

Examples of suitable carrier materials include, but are not limited to: bandages, gauze, wound dressings, adhesive bandages, scaffold, hydrogels, in particular hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid as well as gelatin. The above carrier materials may include alginate (as a thickener or stimulant), buffers to control pH such as disodium hydrogen phosphate/sodium dihydrogen phosphate, agents to adjust osmolarity such as sodium chloride, and stabilizers such as EDTA.

In some specific examples, the carrier material is a hydrogel or a scaffold.

The term “hydrogel” as used herein refers to a three-dimensional (3D) network of hydrophilic polymers. Hydrogels can generally absorb a large amount of fluid and while maintaining the structure due to chemical or physical cross-linking of individual polymer chains. In equilibrium, hydrogels are typically 60-90% fluid and only 10-30% polymer. In some examples, the water content of a hydrogel is approximately 70-80%. Hydrogels are particularly useful because of the inherent biocompatibility of the crosslinked polymer network. Hydrogels can be prepared by crosslinking hydrophilic biopolymers or synthetic polymers. Examples of hydrogels formed by physical or chemical crosslinking of hydrophilic biopolymers include, but are not limited to, hyaluronans, chitosans, alginates, collagen, dextran, pectin, carrageenan, polylysine, gelatin, or agarose. These materials consist of high molecular weight framework chains made with linear or branched polysaccharides or polypeptides.

Hydrogels closely resemble the natural living extracellular matrix. Hydrogels can also be made to be degradable in vivo by incorporating PLA, PLGA, or PGA polymers. Furthermore, hydrogels can be modified with fibronectin, laminin, vitronectin, or, for example, with RGD for surface modification, which can promote cell adhesion and proliferation. Furthermore, alteration of molecular weights, block structures, degradable linkages, and crosslinking modes can influence the strength, elasticity, and degradation properties of hydrogels.

Hydrogels can also be modified with functional groups for the covalent attachment of a variety of proteins (eg, collagen) or compounds such as therapeutic agents. Therapeutic agents that can bind to the matrix include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, anti-arrhythmic, antituberculous, antitussive, antiviral, cardioactive, cathartic, chemotherapeutic agents, a colored or fluorescent imaging agent, corticosteroids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary antiinfectives, vasoconstrictors, vasodilators, vitamins, xanthine derivatives, and the like. The therapeutic agent may also be other small organic molecules, naturally isolated entities or their analogues, organometallic agents, chelated metals, or metal salts, peptide-based drugs, or binding or targeting agents to a peptide or non-peptide receptor. Molecules that can be incorporated into the hydrogel matrix include, but are not limited to, vitamins and other nutritional supplements; glycoproteins (eg, collagen); fibronectin; peptides and proteins, carbohydrates (both simple and complex); proteoglycans; antigens; oligonucleotides (sense and antisense DNA and/or RNA); antibodies (for example, against infectious agents, tumors, drugs, or hormones); and gene therapy reagents.

A number of classifications of hydrogels have been reported. For example, hydrogels can be divided into those formed from natural polymers and those formed from synthetic polymers. Depending on the ionic charges on the bound groups, hydrogels may be cationic, anionic, or neutral. Hydrogels can also be classified as inert, physical, chemical, or biochemical hydrogels. Inert hydrogels are inactive to normal chemical or biological processes, and they are resistant to degradation, and not absorbed by the body. Physical hydrogels can undergo a transition from liquid to a gel in response to a change in environmental conditions such as temperature, ionic concentration, pH, or other conditions such as mixing of two components. Chemical hydrogels use covalent bonding that introduces mechanical integrity and degradation resistance compared to other weak materials. In biochemical hydrogels, biological agents like enzymes or amino acids participate in the gelation process. It is also possible to divide hydrogels into groups based on their structure: amorphous, semicrystalline, crystalline, and hydrocolloid aggregates.

In some examples, the hydrogel is an inert hydrogel. In some other examples, the hydrogel is physical hydrogel, in particular a thermoresponsive hydrogel. Thermoresponsive hydrogels use temperature as external stimulus to show solution-gel transition and most of the thermoresponsive polymers can form hydrogels around body temperature. Various inert and thermoresponsive hydrogels are commercially available. For example, Vaseline is an inert hydrogel, and Pluronic F-127 is a thermoresponsive hydrogel.

A scaffold may be infused with, coated with, or comprised of cells, growth factors, extracellular matrix components, nutrients, integrins, or other substances to promote cell growth. The scaffold may also serve as a carrier material for the pharmaceutical composition disclosed herein. Scaffolds may be formed from biologic or synthetic scaffold materials, and are used in the field of tissue engineering to support protein adhesion and cellular ingrowth for tissue repair and regeneration. The current state of the art in scaffold technology relies upon the inherent characteristics of the surrounding tissue space for the adsorption of proteins and migration of cells. Nonlimiting examples of suitable scaffold materials include extracellular matrix proteins such as fibrin, collagen or fibronectin, and synthetic or naturally occurring polymers, including bioabsorbable or non-absorbable polymers, such as polylactic acid (PLA), polyglycolic acid (PGA), polylactide-co-glycolide (PLGA), polyvinylpyrrolidone, polycaprolactone, polycarbonates, polyfumarates, caprolactones, polyamides, polysaccharides (including alginates (e.g., calcium alginate) and chitosan), hyaluronic acid, polyhydroxybutyrate, polyhydroxyvalerate, polydioxanone, polyorthoesthers, polyethylene glycols, poloxamers, polyphosphazenes, polyanhydrides, polyamino acids, polyacetals, polycyanoacrylates, polyurethanes (e.g., GranuFoam®), polyacrylates, ethylene-vinyl acetate polymers and other acyl substituted cellulose acetates and derivatives thereof, polystyrenes, polyvinyl chloride, polyvinyl fluoride, poly(vinylimidazole), chlorosulphonated polyolefins, polyethylene oxide, polyvinyl alcohol, Teflon®, and nylon. The scaffold can also comprise ceramics such as hydroxyapatite, coralline apatite, calcium phosphate, calcium sulfate, calcium carbonate or other carbonates, bioglass, allografts, autografts, xenografts, decellularized tissues, or composites of any of the above. In some examples, the scaffold may comprise collagen (e.g., Biostep® or Promogran® scaffolds), polylactic acid (PLA), polyglycolic acid (PGA), polylactide-co-glycolide (PLGA), a polyurethane, a polysaccharide, an hydroxyapatite, or a polytherylene glycol. Additionally, the scaffold can comprise combinations of any two, three or more materials, either in separate or multiple areas of the scaffold, combined noncovalently or covalently (e.g., copolymers such as a polyethylene oxide-polypropylene glycol block copolymers, or terpolymers), or combinations thereof.

In another aspect, there is provided the pharmaceutical composition as disclosed herein for use in therapy.

In one aspect, there is provided method of treating a wound, the method comprises administering a pharmaceutically effective amount of the pharmaceutical composition as disclosed herein to a subject in need thereof.

The present disclosure also provides a method for promoting regeneration of epithelial tissue in a subject. In some examples, the regeneration of epithelial tissue is promoted at the site of a wound in the subject, and, thus, contributes to the promotion of wound healing in the subject.

The present disclosure also provides the pharmaceutical composition as disclosed herein for use in treating a wound in a subject. The present disclosure also provides the pharmaceutical composition as disclosed herein for promoting regeneration of epithelial tissue in a subject. Also provided are use of the pharmaceutical composition as disclosed herein in the manufacture of a medicament for treating a wound in a subject. Also provided are use of the pharmaceutical composition as disclosed herein in the manufacture of a medicament for promoting regeneration of epithelial tissue in a subject.

As used herein, the term “treating” includes reducing or alleviating at least one adverse effect or symptom of a disease or disorder. As used herein, “pharmaceutically effective amount” refers to an amount of the pharmaceutical composition that is sufficient to bring about a beneficial or desired clinical effect. Said amount could be administered in one or more administrations. However, the precise determination of what would be considered an effective amount may be based on factors individual to each patient, including, but not limited to, the patient's age, the size of wound, the type of wound, the severity of the wound, route of administration of the pharmaceutical composition, etc. This amount may be readily determined by the skilled person, based upon known procedures, including clinical trials, and methods disclosed herein.

As used herein, the term “subject” includes warm-blooded animals, preferably mammals, including humans. In some specific examples, the subject is a primate. In one specific example, the subject is a human.

In some examples, the subject is a subject suffering from, or thought to suffer from, an underlying condition or disease. In some other examples, the subject is suffering from or thought to suffer from cancer. In some further examples, the subject is suffering from, or thought to suffer from, diabetes. In yet some other examples, the subject is undergoing further treatment or has undergone further treatment, whereby the treatment is, but is not limited to, chemotherapy, chemoprevention, radiation therapy, immune suppressive therapy, steroid treatment, and the like.

As used herein, the term “wound” refers to an injury to a body that typically involves laceration or breaking of a membrane, for example, such as the skin. Wounding may also include damage to underlying tissues, and is, in most cases, usually a result of an external, physical force on the body.

In some examples, the wound is characterized as being slow healing, or nonhealing.

A nonhealing wound, for example, is a wound that does not heal according to an orderly set of stages and in a predictable amount of time the way most wounds do; wounds that do not heal within three months are often considered to be non-healing. Wounds can display a spectrum of healing rates, whereby acute and non-healing wounds lie at opposite ends of the spectrum.

A possible reason for the occurrence of a nonhealing or slow healing wound can be, for example, due to preexisting and/or underlying conditions or diseases, or because a subject is undergoing further treatment, whereby the further treatment results in impaired wound healing. These conditions or diseases may be pathological or non-pathological and can aggravate or exacerbate wound healing by being present in the subject. Examples of such conditions and/or diseases are, but are not limited to, cancer, diabetes (type I and type II), skin disorders, autoimmune disorders, inflammatory disorders (both internal and external) of the epithelial lining, the dermis and/or the sub-dermis, eczema, and the like.

Examples of wound include but are not limited to, chronic wounds, acute wounds, traumatic wounds, sub-acute wounds, and dehisced wounds, wounds caused by burns, partial-thickness burns, ulcers (such as diabetic, pressure, or venous insufficiency ulcers), flaps, and grafts. In one example the wound is caused by burns or a chronic wound.

The pharmaceutical composition as disclosed herein may be applied to a wound through direct topical application. Alternatively, the pharmaceutical composition may be applied to a carrier material, which is then applied to the wound. Such methods may include application of the pharmaceutical composition to a bandage, gauze, or dressing to be applied to the wound. The pharmaceutical composition provided herein may also be added to other known compositions for treating wounds.

The term “topical” application refers to application to skin, dermis or tissue site, and application to such tissue sites may include application adjacent to or within the tissue site.

The term “tissue site” as used herein broadly refers to a wound or defect located on or within tissue, including but not limited to, bone tissue, adipose tissue, muscle tissue, neural tissue, dermal tissue, vascular tissue, connective tissue, cartilage, tendons, or ligaments.

Other than topical application, the pharmaceutical composition as disclosed herein can also be administered to a subject in need thereof via other routes. Examples of modes of administration include but are not limited to, intravenous, intravascular, intramuscular, subcutaneous, intracerebral, intraperitoneal, soft tissue injection, surgical site, arthroscopic site, and percutaneous insertion, eg, by direct injection, cannulation, or catheterization. Any administration can be a single application of the pharmaceutical composition or multiple applications. Administrations can be at a single site or at more than one site in the subject to be treated. Multiple administrations can occur at essentially the same time or separate over time.

It is also shown in the present disclosure that the Agrin fragment or derivative of as described herein promotes collective keratinocyte migration. It is also shown that the Agrin promoted collective keratinocyte migration is achieved by engaging MMP12 as a downstream effector. Since wound re-epithelialization begins after keratinocytes at the wound margins becomes activated for migration, the present disclosure also provides a method for promoting wound re-epithelialization, the method comprises administering an Agrin fragment or derivative thereof as disclosed herein.

As used herein, the term “re-epithelialization” refers to the process of creating a new barrier between wound and environment through epithelial cell migration. The cellular and molecular processes involved in the initiation, maintenance, and completion of re-epithelialization are essential for successful wound closure.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

It will be understood by those skilled in the art that a wide variety of methods and techniques known in the art may be used in carrying out certain embodiments of the present invention.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

Materials and Methods

Antibodies, siRNAs, and Reagents

The following antibodies were used in the study: Mouse monoclonal anti-Agrin (D-2), Santa Cruz Biotechnology, Cat #sc-374117; RRID: AB_10947251; Human Agrin clone PIF12 (in house generated); Rabbit polyclonal Agrin antibody, Novus Biologicals, Cat #NBP1-90209, Mouse monoclonal anti-Integrin β1, Abcam, Cat #ab24693; RRID: AB_448230; Anti-Integrin Beta1, activated, Clone HUTS-4, Cat #MAB2079Z, RRID:AB_2233964; Mouse monoclonal anti-β Actin (C4), Santa Cruz Biotechnology, Cat #sc-47778; RRID: AB_2714189; Rabbit polyclonal anti-GAPDH (FL-335), Santa Cruz Biotechnology, Cat #sc-25778; RRID: AB_10167668; Phospho-Myosin Light Chain 2 (Ser19) Antibody #3671, RRID:AB_330248, Anti-Myosin light chain (phospho-S20) antibody (ab2480), RRID:AB_303094; Myosin Light Chain 2 Antibody #3672, RRID: AB_330278; MMP-12 Antibody clone G-2, Cat #sc-390863; Anti-MMP12 antibody, Abcam, Cat #ab137444; MMP12 Polyclonal Antibody, Thermo Fisher Scientific, Cat #PA5-27254, RRID: AB_2544730; Rabbit polyclonal anti-CD31, Abcam, Cat #ab28364; RRID: AB_726362; Goat anti-rabbit IgG-HRP, Santa Cruz Biotechnology, Cat #sc-2030; RRID: AB_631747; Goat anti-mouse IgG-HRP, Santa Cruz Biotechnology, Cat #sc-2005; RRID: AB_631736; Goat anti-mouse IgM-HRP, Santa Cruz Biotechnology, Cat #sc-2973; RRID: AB_650513; Goat anti-Rabbit IgG (H+L) Alexa Flour 488 Invitrogen, Cat #A11034; Goat anti-mouse IgM Alexa Flour 594, Invitrogen, Cat #A21044. The following siRNAs were used in this study: human Agrin Stealth siRNAs (Set of 3) HSS139721, HSS180123, HSS180124; mouse Agrin Stealth siRNAs (Set of 3) MSS201833, MSS201834, MSS201835 (Thermo Fisher Scientific), human Agrin smartpool (Cat #L-031716-00-0050), human MMP12 (Cat #L-005954-00-0050) from Dharmacon. The specific siRNA sequences are listed in Table 3. Lipofectamine RNAimax (Invitrogen) was used for siRNA transfections following the manufacturer's recommended guidelines. Fibronectin was obtained from Gibco, and Advanced Biomatrix. Rat tail collagen type 1 was from Corning. Alexa-488 conjugated F-actin phalloidin was from Thermo Fisher Scientific. Blebbistatin and Pluronic F127 was obtained from Sigma and MMP408 from Merck Millipore, respectively. Aquaphore was obtained from Beirsdorf AG while Vaseline was from Unilever. Dimethylsulfoxide was from Kanto Chemical, co., Inc., Cat #10378-00. CellTiter 96® AQueous One Solution Cell Proliferation Assay (MTS), Promega Cat #G3580, was used for proliferation assays. 5-bromo-2′-deoxyuridine-BrdU (#Cat ab142567) and Anti-BrdU antibody (Cat #ab6326) were purchased from Abcam.

Mice

Six to eight to week old female experimental ICR (nomenclature: IcrTac:ICR) mice purchased from InVivos were used for punch-biopsy wound healing in vivo assays. In the ex-vivo mouse skin explant assay, random ratios of male and female new born ICR mouse pups were used. Wherever possible, the experiments were performed taking into consideration an ethical and reductionist approach of animal usage. Animal studies were not performed in a blinded fashion. The animals were randomly assigned to different experimental groups. The number of animals used for each experiment is indicated in the legend as n=x mice per group. All animal experiments performed were done in accordance with experimental protocols reviewed by the Biological Resource Center (BRC), Agency for Science Technology and Research (A*STAR) under strict compliance to the Institutional Animal Care and Use Committee (IACUC) guidelines for ethical use of animal models in biological research.

Cell Lines and Human Skin Explants

Human epidermal keratinocyte cell line HaCaT was maintained in Dulbecco's modified Eagle's Medium (DMEM) (Gibco) containing 10% fetal bovine serum (FBS) with Penicillin and Streptomycin (Gibco Cat #15140148) antibiotics. The human foreskin normal fibroblasts (BJ) obtained from ATCC (ATCC® CRL-2522™) were passaged in DMEM (Gibco) with antibiotics. Normal primary adult epidermal keratinocytes (HEK) purchased from CELL Applications, Inc., (Cat #C-12003) were maintained in manufacturer provided keratinocyte growth medium as recommended. Primary keratinocytes from C57BL mouse strain were purchased from CellBiologics (Cat #C57-6066K) and maintained as per manufacturer recommended Epithelial cell growth medium (Cat #M6621, CellBiologics). The mouse primary dermal fibroblasts were isolated from C57BL mouse strain (Cat #m-GFP-6067), and cultured in complete fibroblast medium (CellBiologics, Cat #M2267). Epiderm full-thickness (EPIDERM-FT™) human skin explants on a basement membrane bearing 3 mm punch wounds were purchased from MatTek Lifesciences and maintained using the company supplied medium. All cells were propagated at standard culture conditions of 37° C. and 5% CO₂.

RNA Interference and Knockdown In Vitro and In Vivo

The desired siRNAs were dissolved as per the manufacturer's recommended buffers. For in vitro studies, 50 nM of control or targeted siRNA was mixed with 5 μl of Lipofectamine RNAimax (Thermo Fisher Scientific), and incubated for 30 min at room temperature. The siRNA: lipofectamine mixture was added to 250 μl of reduced growth factor medium. The knockdown was verified by RT-PCR or Western blot after 72 h. For in vivo and ex vivo RNAi, 75 nM of stealth siRNAs were mixed with 8 μl of Lipofectamine RNAiMAX (Thermo Fisher Scientific) for 30 min at room temperature. Subsequently, the siRNA mix was slowly incorporated into a 1:1 mixture of Aquaphore gel in Phosphate buffered saline (PBS) for topical delivery. For treatment with skin explants, the siRNA lipofectamine mix was added to Aquaphore as 1:1 mixture at day 0 post-wounding and incubated for the indicated number of days. For efficient knockdown, the topical siRNA ointment was re-applied on day 4 post-wounding. For the explants that were rescued with sAgrin, an Aquaphore mix containing 20 μg protein was added on days 2, 4 and 6 post-wounding.

Recombinant Agrin Expression and Purification

The C-terminus fragment containing 8 amino acid ‘Z8’ insert and LG3 domain (AgrinZ8LG3, having the sequence of DTLAFDGRTFVEYLNAVTESELANEIPVEKALQSNHFELSLRTEATQGLVLWSGKA TERADYVALAIVDGHLQLSYNLGSQPVVLRSTVPVNTNRWLRVVAHREQREGSLQ VGNEAPVTGSSPLGATQLDTDGDCALWLGGLPELPVGPALPKAYGTGFVGCLRDVVV GRHPLHLLEDAVTKPELRPCPTP (SEQ ID NO: 7)) was cloned into a pET28 vector containing His-ta and expressed in Escherichia coli (strain BL21-DE3). The bacteria were transformed with the plasmid. A single colony was inoculated in TB medium containing antibiotics and induced with IPTG for 16 h at 15° C. The cells were harvested by centrifugation and cell pellets were lysed in lysis buffer followed by sonication. The resultant supernatant containing Agrin protein fragment was purified using gel filtration chromatography via Superdex75 columns in imidazole buffer, and sterilized by passing through a 22 μm low-protein binding filter and was dissolved in PBS solution.

Punch-Biopsy Wound Healing Models

The mice were housed under standard conditions of 21° C. and a 12 h light-dark cycle with free access to food. Subsequently, the mice were intraperitoneally (i.p.) anesthezised using Ketamine 100 mg/kg and Xylazin 10 mg/kg diluted in 100 ml of saline solution. The fur was shaved from the base of the neck towards the back on the entire shoulder region. The skin was wiped with an alcohol swab and 10% povidone-iodine (Betadine) antiseptic solution. Circular symmetrical 4 mm wounds were inflicted on either side of the midline in the shoulder region using a sterile 4 mm punch biopsy needle (Integra Miltex, Integra York, P.A., Inc.). The wound skin was carefully removed using a scalpel and a pair of scissors to generate a full-thickness wound that were left open for topical administrations during the analysed time periods. For splinted wound healing models, a 10 mm transparent donut shaped nylon sheet (Grace Bio-Laboratories, Bend, Oreg.) was placed around the 4 mm wound. The splint was placed with the wound at the center, glued to the skin by adhesive (Krazy Glue®; Elmer's Inc.), and covered by Tegaderm dressing. The wounds were treated with different formulations based on either petroleum jelly (Vaseline), a thermoresponsive hydrogel (Pluronic F127-Sigma) or a commercially available skin ointment (Aquaphore-Beiersdorf, Inc.). For Vaseline based ointment cream preparation, 10 mg per cm² vaseline ointment was mixed with filtered PBS solutions containing the indicated amounts of Agrin (100 μg, 200 μg or 500 μg) and 100 μg BSA (w/w) were used and applied topically every two days. For siRNA mediated knockdown experiments, 75 nM of indicated siRNAs were incubated with lipofectamine in PBS. Subsequently, a 1:1 mixture of PBS (containing the siRNAs): Aquaphore was applied topically on the wound region every two days. A topical formulation comprising of 20% (w/w) Pluronic F127 (Sigma) was used to dissolve in sterile and 0.22μ filtered solution containing a final concentration of 200 μg/ml sAgrin or BSA. This mixture was dissolved overnight at 4° C. to ensure that liquid phase is achieved. The mixture forms a homogenous gel at temperatures above ˜20° C. and was dispersed topically to cover the wound. Wound closure was assessed using a Vernier caliper every alternate day. For both models, the animals were caged individually throughout the observed time-points of healing at the institutional animal facility. All wound healing animal experiments performed were done in accordance with experimental protocols reviewed by the Biological Resource Center (BRC), Agency for Science Technology and Research (A*STAR) under strict compliance to the Institutional Animal Care and Use Committee (IACUC) guidelines for ethical use of animal models in biological research.

Mouse Skin Explant Assay

Three days old ICR strain mouse pups were sacrificed and the hind skin on either side of the midline in the shoulder region was washed in 70% ethanol and excised using scalpel. The excised skin was washed in 70% ethanol and placed (dermis side down) on the pre-made gel substrates of defined stiffness, either in the absence or presence of 20 μg/ml sAgrin. The explants were allowed to adhere for 2 h before adding the keratinocyte culture medium (DMEM) containing 300 μM Ca²⁺ as described previously³¹. The keratinocyte outgrowth and the skin tissues regularly imaged under an Axiovert-200 inverted microscope and were subsequently fixed in 4% paraformaldehyde and immunostained for Keratin 17 antibody.

Substrate Stiffness Manipulations and Micropatterning

Tissue culture plates were coated with Col-T-gel (Fischer Scientific) of stiffness ranging from 0.8 KPa (soft) to 30 KPa (stiff) as per manufacturer recommendations and allowed to solidify for 40 min at 37° C. inside tissue culture incubators. Some experiments were performed with cells seeded on the poly-hydrogel plates of defined stiffness: 0.2 kPa and 16 kPa soft and hard polyhydrogels, respectively (CytoSoft, Advanced Biomatrix, Inc.,). The determination of stiffness of silicone substrates were done by the manufacturer as per previously published protocols. For certain experiments, indicated amounts of sAgrin was incorporated into the 0.8 KPa collagen gels before gelation process. Upon gelation, trypsinized cells or excised mouse skin explants were plated onto the gels of different stiffness either alone or containing sAgrin. The cells/tissues were incubated under standard culture condition with 500 μl recommended culture medium for the indicated days. The crossbow shaped micropatterns with fibronectin coated islands were generated by microlithography on a 19.5×19.5 mm coverslide from Cytoo, Inc., France. The micropatterns had surface areas of 800μ² or 1600μ², respectively. Fifty thousand cells after initial siRNA treatment was placed on the micropatterns for 4-6 h before processing them for immunofluorescence. For some experiments, 10 μg/ml sAgrin was added on the slides 18 h prior to the addition of cells.

3D-Stiffness Dependent Keratinocyte Fibroblast Co-Culture Migration Assay

A 3D in-vitro wound healing model was created using collagen constructs of defined stiffness modifying a previously published protocol. Collagen constructs were made with Col-T-gel (0.8 KPa-soft) or (30 KPa-stiff) as per manufacturer's recommended protocol containing 300,000 primary mouse dermal fibroblasts (DFs). For some experiments, siControl and siAgrin treated DFs were incorporated within soft or stiff collagen constructs. The constructs bearing fibroblasts were allowed to solidify for 45 min inside incubator. A second layer of collagen matching the stiffness of the underneath layer was added containing 350,000 of primary mouse keratinocytes (KRTs) and the construct was allowed to solidify for 3-4 h. After removing excess media, the constructs were washed gently with 1×PBS twice. An upside down 2μ pipette tip was inserted and gently rotated at the center of the constructs to create a circular wound. The collagen and cells was quickly removed from the wound area and replaced with 40 μl of soft or stiff Col-T-gel matching the consistency and set-up of the constructs. The resultant wounded constructs were placed inside the incubator and imaged after 30 min for day 0 time-points. The migratory keratinocytes were imaged till day 5 post-wounding.

Western Blot Analysis

Indicated cells post-manipulation were washed twice with cold Phosphate buffered saline (PBS) and lysed with cold 1% NP-40 lysis buffer supplemented with 1× protease inhibitor cocktail (Roche Applied Biosciences) for 15 mins at 4° C. The cell lysate was centrifuged at 13,000 rpm for 15 min. This was followed by protein estimation using Bradford reagent. Subsequently, 40-50 μl of total protein was mixed with an equal volume of 2× Laemmli sample buffer and heated at 95° C. for 5 mins. This was followed by resolution with SDS-PAGE gel. The resolved proteins were transferred onto nitrocellulose membrane and blocked in 5% skimmed milk reconstituted in 1×PBS containing 0.1% Tween-20, and probed overnight with the respective primary antibody. The membrane was then washed with 1×PBS supplemented with 0.1% Tween-20: three times at 15 min intervals. This was followed by 1 h incubation in conjugated horseradish peroxidase HRP secondary antibody (Santa Cruz Biotechnology). Post-incubation, the blot was again washed three times as above and then overlaid with enhanced chemiluminescence (ECL) substrate (Pierce/Bio-Rad) and visualized on X-ray film by image processor or digitally by Chemidoc analyzer (Bio-Rad). The density of the various bands was quantified using the Image-J software.

Immunofluorescence and Confocal Microscopy

Cells were cultured on eight-well chamber slides or coverslips over-night. Cells were then washed twice with PBS and fixed for 15 min with 4% paraformaldehyde. Subsequently, the cells were permeabilized for 15 min with 0.1% Triton X in Phosphate buffered saline containing 1 mM Ca⁺² and 1 mM Mg⁺² (PBSCM) at room temperature. The permeabilized cells were incubated with indicated antibodies in fluorescent dilution buffer (FDB) for 1-2 h at RT or overnight at 4° C., followed by 5 washes with PBSCM and incubation with secondary antibody; Alexa Fluor (Thermo Fisher Scientific) for 1 h at RT. Slides were again washed five times with PBSCM and mounted with Vectashield medium containing DAPI. The stained cells were then imaged by a Zeiss confocal microscope. The images were processed and analyzed by Zen blue software. Intensity measurements were acquired using tool selection parameter in image analysis within the Zen blue software.

Histology and Immunofluorescence-Histology

Human skin equivalents and mice skin tissues were harvested, embedded, and subjected to routine histology using Hematoxylin and Eosin. For immunostaining, the tissues were fixed for 24 h in 10% neutral buffered formalin solution. Subsequently, the tissues were sectioned using a microtome and subjected to antigen-retrieval at pH 9 or pH 6 (for the indicated antibodies). Primary antibodies were used at a concentration of 1:50 dilution, while the respective Alexa-488 and Alexa-594 secondary antibodies (Thermo Fisher Scientific) were used at a concentration of 1:100 and 1:500, respectively in fluorescent dilution buffer. The slides were mounted in a VECTASHIELD Hardset™ antifade mounting medium containing DAP. Quantification of collagen as a marker for ECM deposition was done by picrosirius red staining of paraffin embedded mouse skin tissues, as per previously published protocol.

In Vitro Wound-Healing Assay

A confluent monolayer of control and Agrin depleted cells in a 6-well plate was subjected to a unidirectional scratch using a 20 μl pipette tip. This was followed by washing with 1×PBS at room temperature and incubation in complete culture media at 37° C. and 5% CO₂ with or without sAgrin used as indicated. Phase contrast images of the wound area were taken periodically at the indicated time-points. For some wound-healing migration assays, keratinocytes were cultured on either soft or stiff substrates within a stencil barrier (Nalge Nunc., Inc) for 18 h. Upon removal of the barrier, the cells were allowed to migrate and imaged using an Axiovert 200 inverted microscope at indicated time-points.

Traction Force Microscopy

Substrate preparation—Two-part silicone elastomer, DOWSIL CY52-276 (Dow Inc.), is first mixed thoroughly in a 1:1 weight ratio. The mixture is then poured into a 35 mm glass-bottom culture dish (iwaki 3930-035, Asahi Techno Glass Corporation, Japan) placed on a level surface, to a thickness of ˜500 μm. After pre-curing overnight, the silicone coated culture dish is further baked for 2 hours at 80° C. to fully cure. To maximize subsequent microsphere attachment, the silicone film is pre-treated with (3-aminopropyl) triethoxysilane (A3648, Sigma-Aldrich). To silanize, a 1 ml solution containing 96% v/v, 2% v/v APTES and 1% v/v acetic acid (A6283, Sigma Aldrich) is added to the glass-bottom culture dish, covering the whole silicone film, and left for 10 minutes to react. The silicone film is then twice rinsed with 96% v/v ethanol and followed by a quick dip in Milli-Q water to remove all the solution: removing solution from the film this way is preferred over blow-drying with nitrogen gas because the latter may introduce dust to the surface which subsequently affects the uniformity of attached microsphere distribution. Following this, the culture-dish is baked at 80° C. for another 2 hours to promote siloxane bond formation. For the physisorption of fluorescent latex microspheres on to the silicone film, 6 μl of 100 nm orange fluorescent (540/560) carboxylate-modified microspheres (F8800, ThermoFisher Scientific) is first diluted in 10 ml of Milli-Q water and sonicated for 10 minutes. The diluted microspheres are then passed through a 0.22 μm filter directly into a 15 ml falcon tube containing 500 μl of 500 mM MES buffer, pH 6.0 (M3671, Sigma Aldrich). The microspheres are then sonicated again for 10 minutes before being added to cover the APTES silanized silicone film within the culture dish previously prepared. The solution was left for 5 minutes to allow the microspheres to attach. Then, to remove the solution, the culture dish is carefully and quickly tipped so that all the solution is removed in a single action, as the solution's surface tension would decouple microspheres if allowed to flow back. The microsphere coupled culture dishes are then baked at 80° C. for 2 hours and typically used within a week. To facilitate the attachment of adherent cells, the microsphere coupled silicone films are coated with human blood plasma fibronectin (10838039001, from Sigma Aldrich) with a concentration of 50 μg/ml to a surface density of 5 μg/cm². The stiffness of silicone substrates used for TFM were determined by atomic force microscopy as per previously established protocols as described in the section below.

Traction force imaging—A Nikon Biostation IMQ is used to live-cell image the HACAT cell-sheet migration and microsphere displacement over 24 hours with 10-minute intervals, at 37° C. and 5% CO₂. Both phase contrast and epifluorescence images were acquired using the internal 20× objective (0.5 NA) and 1.3-megapixel monochrome camera. The light source for the epifluorescence is the Intensilight Hg Pre-Centred Fibre Illuminator and orange microspheres are imaged using a Texas Red filter set. To account for z-drift, 2-3 planes 1 μm apart either side of the focal plane is typically taken in a z-stack. Three independent samples are imaged for each condition, and for each independent sample 3-4 locations are randomly selected and tracked.

Cell Stiffness by Atomic Force Microscopy (AFM)

The stiffness of the mouse keratinocyte cells was measured using a Nanowizard IV BioAFM system (JPK Instruments, Germany). Confluent mouse kerantinocyte monolayers were cultured on a petri dish in PBS media. The monolayers were scratched across the center of the petri dish using a 100 μl pipette tip and characterised after 4 hrs. Indentations were performed on randomly selected cells at the wound edge with a polystyrene bead of diameter (˜4.5 μm) attached to the end of a cantilever (k=0.03 N/m, Novascan Technologies, Inc., Ames, Iowa) using a force of 3 nN at 1 Hz. More than 60 cells from triplicate experiments were characterized and averaged to evaluate the Young's modulus for each condition. Young's modulus values were calculated using JPK Data Processing Software (JPK Instruments, Germany), which employs Hertz's contact model for spherical indenters (diameter 4.5 μm; Poisson's ratio 0.5) fitted to the extend curves.

Fabrication of Polyacrylamide Block for Collective Traction Force Measurements in a Wound Healing Assay Model

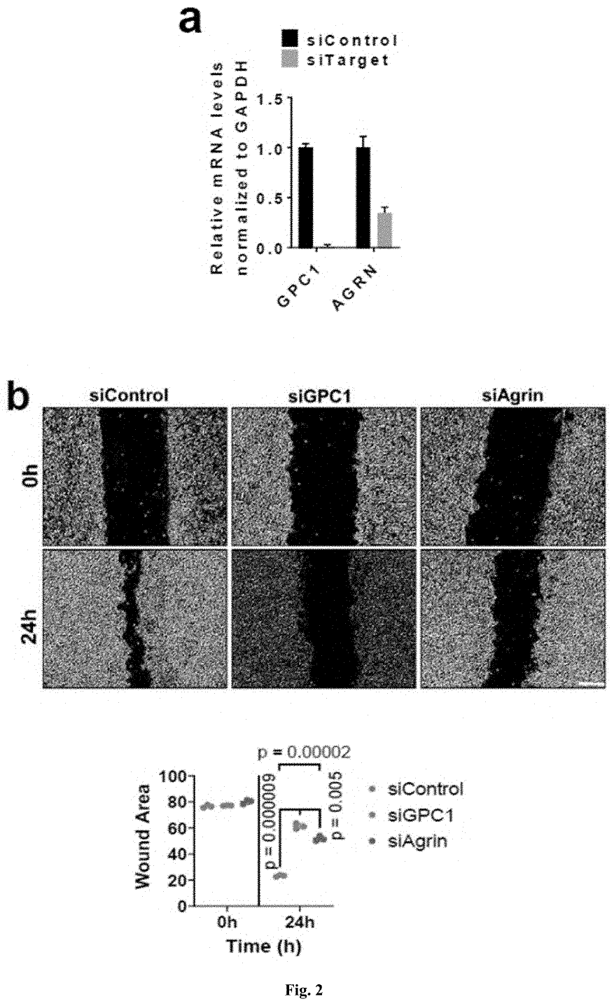

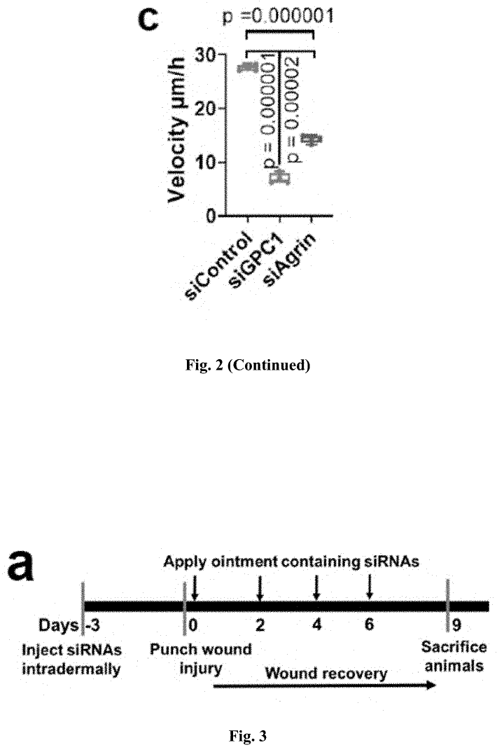

A polyacrylamide precursor mixture is first prepared as follows. To prepare 1 ml of such precursor mixture, 200 μl of 40% w/v aqueous AC (1610140, Bio-Rad), 200 μl of 2% w/v aqueous BIS (1610142, Bio-Rad), 1.5 μl of TEMED (1610800, Bio-Rad), and 583 μl of Milli-Q water are thoroughly mixed together. 500 μl of this mixture is then combined with 8 μl of 10% ammonium persulfate (1610700, Bio-Rad) and quickly pipetted into to a 3D printed mould with a cavity size of 1.2×1.2×0.5 cm. A slightly larger square piece of 3-(trimethoxysilyl) propyl methacrylate (440159, Sigma-Aldrich) silanized glass is then placed over the top. The glass will covalently bind to the polyacrylamide once reaction completes, and acts as a solid support for the block; it will also facilitate adding weights to the block. The silanization procedure for this glass support is similar to that performed for the silicone film in the main text, except TMSPMA is used instead of APTES. The solid hydrogel is removed from the mould after 30 minutes and immersed in Milli-Q water overnight to wash out remaining toxic components. The mould and the finished block are shown in FIG. 2(a) and the intended placement of block in the glass-bottom culture dish is demonstrated in FIG. 0 2(b).

Wound Assay Using Microfabricated Device for Collective Traction Force Microscopy

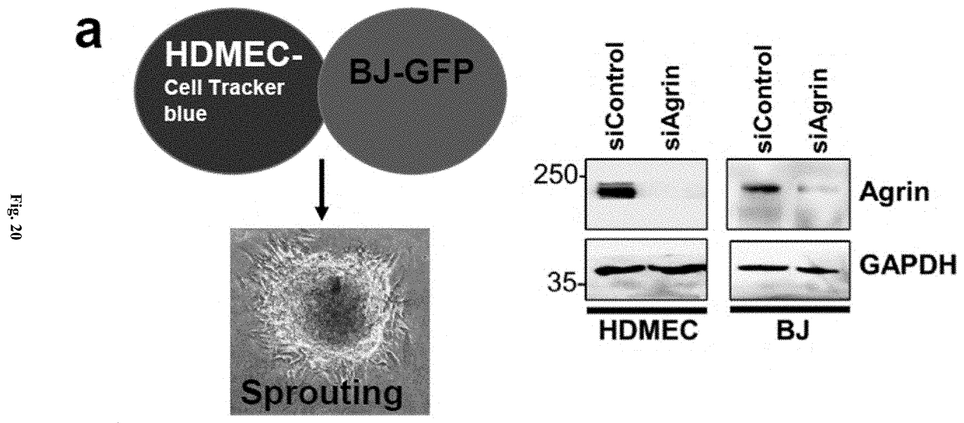

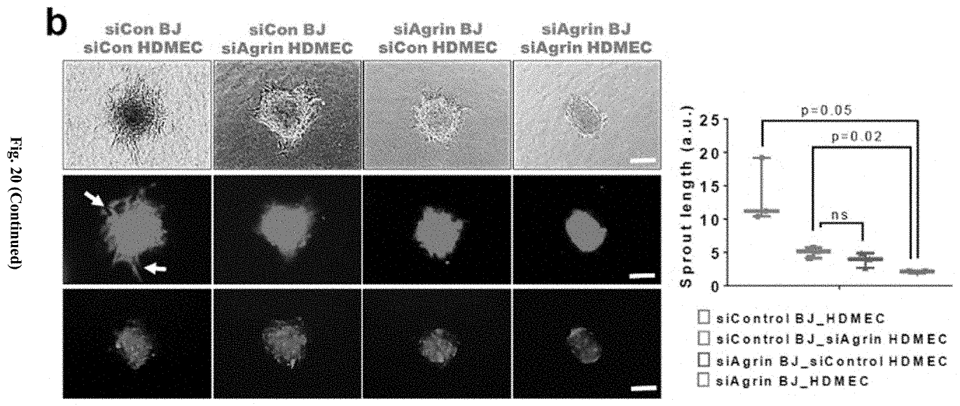

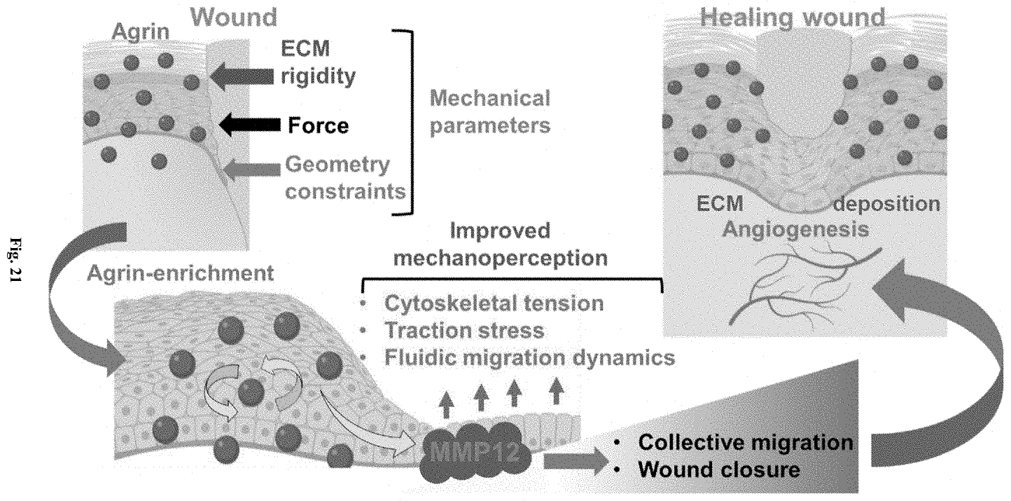

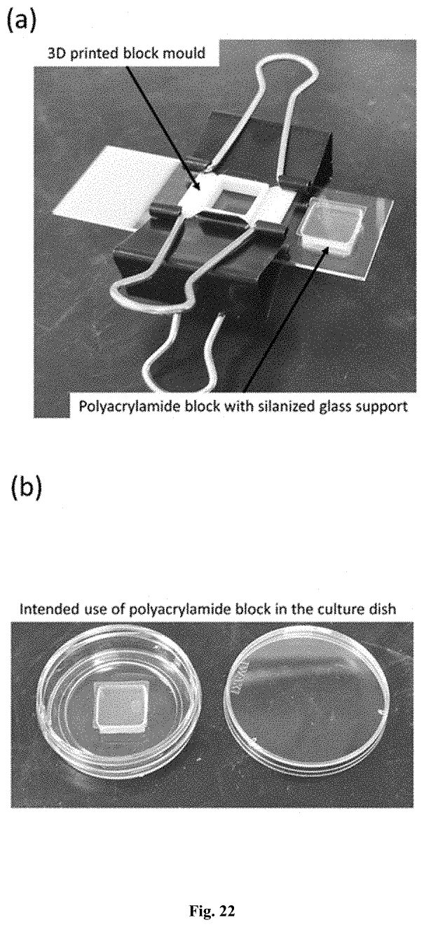

Prior to seeding the HaCaT cells, the 35 mm culture dish containing the ECM coated silicone film is setup with a block of polyacrylamide hydrogel (1.2×1.2×0.5 cm, see also FIG. 22) which will eventually act to create a clean wound in the HaCaT cell sheet. Polyacrylamide hydrogel is used for the fact that it is resistant to protein binding and would therefore prevent cell adhering to it and also prevent the removal of ECM from the silicone surface. After equilibrating the hydrogel block in the culture dish with growth media for 30 minutes, the dish is replaced with fresh media and ˜7×10⁵ cells are seeded evenly around the hydrogel block. The setup is incubated for 24 hours before the hydrogel block is removed, and the cells imaged thereafter (see also FIG. 2).

Force Transmission Via Ligand Conjugated Magnetic Beads

The ligands were conjugated to 4.5 μm epoxide paramagnetic beads (Dynabeads M-450 Epoxy, Thermo Fisher Scientific) according to the manufacturer's manual. Briefly, the beads were washed twice in 0.1 M sodium phosphate buffer, pH 7.4. Subsequently. 8×10⁷ beads were conjugated to 20 μg sAgrin, BSA and FN per in 0.1 M sodium phosphate buffer at pH 7.4 for 16-18 h at 4° C. with gentle rotation throughout. The degree of protein conjugation to the beads was verified by a reducing SDS-PAGE gel analysis using a subset of ligand conjugated beads. The remaining beads were washed and subsequently stored in 0.1% BSA-phosphate buffered saline (PBS) pH 7.4 at 4° C. They were subsequently suspended in medium and added on the cells for 30 min at 37° C. After a brief wash with PBS to remove excess non-adherent beads, the cells were placed under a permanent neodymium magnet (J Magnetics, USA) at a distance of 6 mm apart for another 30 min that allowed a vertical tensile force in the magnitudes of ˜200 pN on the beads. The cells were then fixed in 4% paraformaldehyde, permeabilized and processed for immunofluorescence.

Mathematical Computation of Traction Forces

The displacement fields associated with the fluorescent bead images were computed using PIVLab, an open-source Particle Image Velocimetry MATLAB package, which is based on cross-correlation. Images of the substrate in a stress-free state (i.e. sufficiently long after the addition of 1% v/v sodium dodecyl sulfate (L4509, Sigma Aldrich) to the culture to kill the cells) were used as the reference images to compute these deformations. During cross-correlation, four window passes were used, each of (square) window sizes of 64, 32, 16, and 16 pixels respectively, with 50% window overlap between strides. These computed displacement fields were then used to perform Fourier Transform Traction Cytometry (FTTC) to infer traction forces, using the MATLAB code. The Young's modulus and Poisson's ratio were assumed to be 10 kPa and 0.5 for all of the FTTC calculations, respectively. The L-Curve criterion was used to select the optimal L2 regularisation parameter. More specifically, an L-curve was constructed for the 1^{st}, 10^{th}, 20^{th}, 30^{th}, 40^{th}, 50^{th}, and 60^{th} frame of each sample movie, and then the optimal regularization parameter corresponding to each L-curve was selected using the l-corner function for MATLAB. The median of all the optimal regularization (i.e. the parameters calculated for each frame of every sample) was taken to be the optimal regularisation parameter (found to be 1.26×10⁻⁹), which was then fixed for all samples. Noting that a larger regularization parameter will reduce the magnitude of our computed force fields, this was done to avoid creating artificial differences in the magnitude of the computed traction fields by systematically selecting smaller or larger regularisation parameters for different sample conditions.

Image Stitching

An in-house script based on image cross-correlation was used to stitch neighbouring image fields together to generate a larger traction force field.

Dermal Endothelial Cell Fibroblast 3D-Angiogenesis Assay

One hundred thousand Human Dermal Microvascular endothelial cells (HDMEC) cells were pre-labeled overnight with Cell Tracker Blue CMAC (7-amino-4-chloromethyl coumarin-invitrogen) and mixed with equal amounts of GFP expressing BJ cells. Both the cells were treated with the concerned siRNA and allowed to form co-culture spheroids as per previously established protocols. Images of sprouting ECs was captured 24 h post-embedding and quantified using Sprout morphology plugin from Fiji (Image J) as described previously.

MMP12 Activity Assay

MMP12 activity were monitored via Gelatin and Casein zymography as per previously published protocols. Briefly, control or Agrin depleted HaCaT cells were grown till 80-90% confluency in media without FBS. Supernatants collected from each culture dish were spun down at 10,000 rpm for 5 min and concentrated using Amicon ultra columns. Twenty microliter of medium was mixed with 2×SDS loading buffer and loaded on 10% gelatin or casein gel. The gels were washed with incubation buffer and subsequently stained with Coomassie Brilliant Blue for 1 h. The gels were de-stained for 30 min before imaging using a Chemidoc imager.

Quantitative Reverse Transcription PCR (RT-PCR) and RNA Sequencing

Total RNA was extracted from indicated cells using Qiagen RNeasy mini kit as per the manufacturer's recommended protocol. The total RNA was then reverse transcribed using High-Capacity cDNA reverse transcription kit (Applied Biosystems). The generated cDNA (200 ng) was used as a template for the RT-PCR using the SYBR™Green or Tagman® (Thermo Fisher Scientific) based master mix and probes. The data was normalized to GAPDH as endogenous controls. The RT-PCR primers used in the study for the respective target genes are provided in the Table 1. For RNA-sequencing, the RNA quality was analyzed on a Bioanalyzer instrument (Agilent) using the Agilent RNA 6000 Pico Kit. A total of 4 μg RNA was used for RNA-Sequencing library preparation with the TruSeq Stranded mRNA Library Prep kit (Illumina) based on the manufacturer's instructions. Amplification of libraries was limited to 7 PCR cycles. Purified libraries were quantified by qPCR (KAPA Library Quantification Kit for Illumina, Roche). The Agilent High Sensitivity DNA Kit was used to assess the fragment lengths of a subset of libraries, before combining the rest of the libraries into one pool and further subjecting to a sequencing run on a NextSeq500 (Illumina). The condition of sequencing was represented by a single read high output run at 75 bp read length. Raw reads from fastq files were aligned to the hg38 genome using STAR 2.6.1d. Bam files were sorted and indexed with samtools. The relative number of reads mapping to each gene was quantified with htseqcount on features from the gtf file gencode.v29.annotation.gtf. The raw counts of reads were then imported into EdgeR in R to perform the differential expression analyses using the Gene-ontology, GSEA or other analysis, respectively. Gene set enrichment analysis (GSEA) was performed running the GSEA Pre-ranked tool of the GSEA Software 3.0, using the gene sets from the Molecular Signatures Database (MSigDB) v6.2. Enriched Gene Ontology terms and KEGG pathways were identified using Metascape (https://metascape.org/). Only genes with a false discovery rate (FDR) below 1% and more than 2-fold change in expression between conditions were considered.

TABLE 1
List of RT-PCR primers used

| | Forward/ | | |
|---|---|---|---|
| | Reverse | SEQ | |
| Gene | primer | ID NO | Sequence (5′ to 3′) |
| MMP12 | Forward | 16 | GATGCACGCACCTCGATGT |
| | Reverse | 17 | GGCCCCCCTGGCATT |
| MMP10 | Forward | 18 | GACCCCAGACAAATGTGATCCT |
| | Reverse | 19 | TTCAGGCTCGGGATTCCA |
| MMP-1 | Forward | 20 | CCTAGTCTATTCATAGCTAATCAAGA |
| | Reverse | 21 | AGTGGAGGAAAGCTGTGCATAC |
| GSDMB | Forward | 22 | AAAGCGACCGGCAATATAAA |
| | Reverse | 23 | ATAGCTCAGGACCCGATTTG |
| Duox2 | Forward | 24 | ACGCAGCTCTGTGTCAAAGGT |
| | Reverse | 25 | TGATGAACGAGACTCGACAGC |
| Dkk3 | Forward | 26 | GCGGGAGCGAGCAGATCCAG |
| | Reverse | 27 | GGAAGCTGGCAAACTGGCAG |
| SERPINE2 | Forward | 28 | TCTCATTGCAAGATCATCGCC |
| | Reverse | 29 | CCCCATGAATAACACAGCACC |
| SQLE | Forward | 30 | TTAGAGGAGAAATGCCAAGGAA |
| | Reverse | 31 | CACTGATGAAGGAGGAAGGAAG |
| IFI27 | Forward | 32 | TGGACTCTCCGGATTGACCAAGTT |
| | Reverse | 33 | ATTTGGGATAGTTGGCTCCTCGCT |
| IFI6 | Forward | 34 | CCCATCTATCAGCAGGCTCC |
| | Reverse | 35 | AAAGCGATACCGCCTTCTG |
| ARHGAP26 | Forward | 36 | TAAGAATGCTTCCAGGACCACTC |
| | Reverse | 37 | GCTGTAACATCTGCCGATTTTTC |
| TPD52L1 | Forward | 38 | CTCGGCATGAATCTGATGAATG |
| | Reverse | 39 | TGGCAGTTCCCACGTTATTG |
| EPHB6 | Forward | 40 | TGGACTATCAGCTCCGCTACTATG |
| | Reverse | 41 | GTGGCAGTGTTGGTCTCGC |
| ACAN | Forward | 42 | CTACACGCTACACCCTCGAC |
| | Reverse | 43 | ACGTCCTCACACCAGGAAAC |
| CXCL1 | Forward | 44 | GCGCCCAAACCGAAGTCATA |
| | Reverse | 45 | ATGGGGGATGCAGGATTGAG |
| PDK4 | Forward | 46 | CCGTATTTCTACTCGGATGCTG |
| | Reverse | 47 | TGGCTTGGGTTTCCTGTC |
| AJAP1 | Forward | 48 | GGACTCAGCTCCATGTCTATCC |
| | Reverse | 49 | ACTGAGGTCTCCCCTAAGATCC |
| NPTX4 | Forward | 50 | GAGAAAGTGGTTGAGAGG |
| | Reverse | 51 | GTAATCAACGACGGCAAG |
| ACTB | Forward | 52 | TGACAGGATGCAGAAGGAGATTA |
| | Reverse | 53 | AGCCACCGATCCACACAGA |
| GAPDH | Forward | 54 | CTATAAATTGAGCCCGCAGC |
| | Reverse | 55 | GACCAAATCCGTTGACTCCG |
| AGRN | Forward | 56 | ACACCGTCCTCAACCTGAAG |
| | Reverse | 57 | CCAGGTTGTAGCTCAGTTGC |
| GPC1 | Forward | 58 | CGGCCCCGCCATGGAGCTCC |
| | Reverse | 59 | GGCAGTTACCGCCACCGGGG |
| HSPG2 | Forward | 60 | AGCATGGACGTGGCTGTGCC |
| | Reverse | 61 | GGCGTGCGTGTGTAGCCTGT |
| AGRN | Forward | 62 | AATGGCACGGACTAATTTGC |
| (mouse) | Reverse | 63 | TATGAGGGTTTGTGGGGTGT |
| MMP12 | Forward | 64 | AATGCTGCAGCCCCAAGGAAT |
| (mouse) | Reverse | 65 | CTGGGCAACTGGACAACTCAACTC |
| GAPDH | Forward | 66 | CATCACTGCCACCCAGAAGACTG |
| (mouse) | Reverse | 67 | ATGCCAGTGAGCTTCCCGTTCAG |

Statistical Analysis

The number of biological and technical repeats for each experiment is indicated in the figure legends. For most in-vitro experiments, three biological repeats are performed unless stated differently in the legend. For in vivo experiments, no animals were excluded from analysis and the sample size was not pre-determined using power analysis. The age and sex of animals are mentioned in the methods section. Randomization is not applied for experiments using cell lines. Data are presented as mean+/−s.d. Students ‘t’ test was employed to detect paired comparisons and ANOVA or Multiple t tests were used to compare multiple groups using GraphPad Prism software. The data was considered statistically significant when *p<0.05, **p<0.005, ***p<0.0005, respectively. Data considered insignificant was designated as ‘ns’. No prior statistical tests or assumptions were used to determine the sample size of in vitro, ex vivo and in vivo experiments. Three biological replicates were chosen for in-vitro experiments as it adheres to the commonly held practice in biomedical research.

Results

Skin Wounding Actuates an Agrin-Enriched Microenvironment

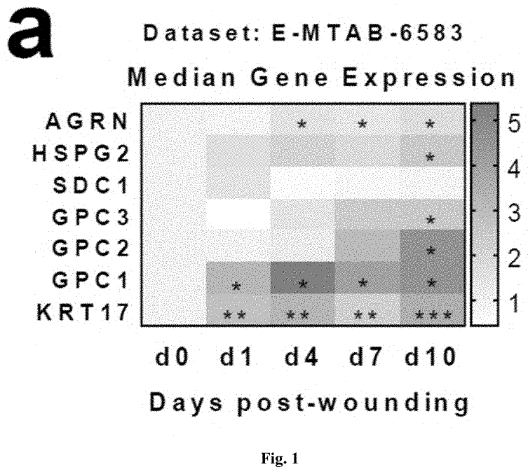

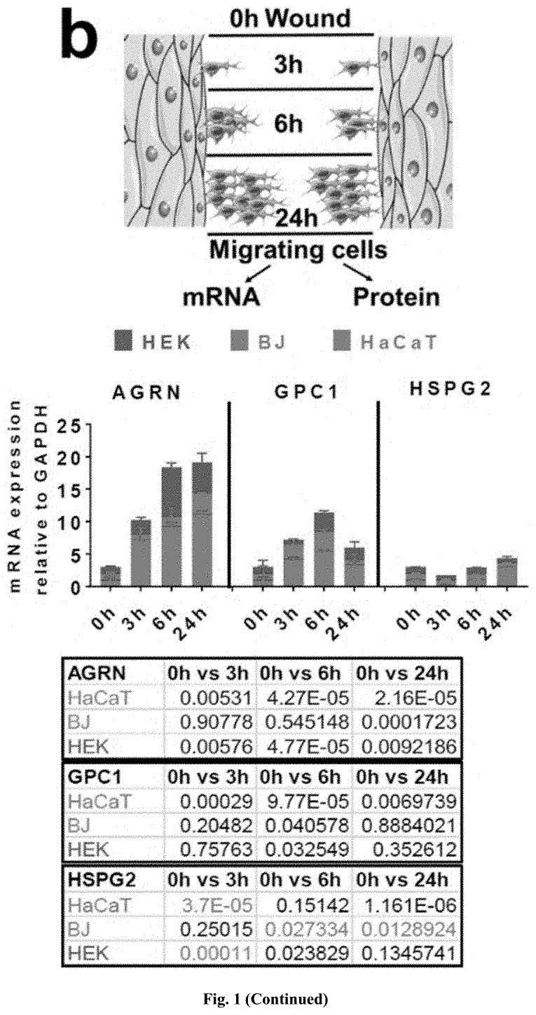

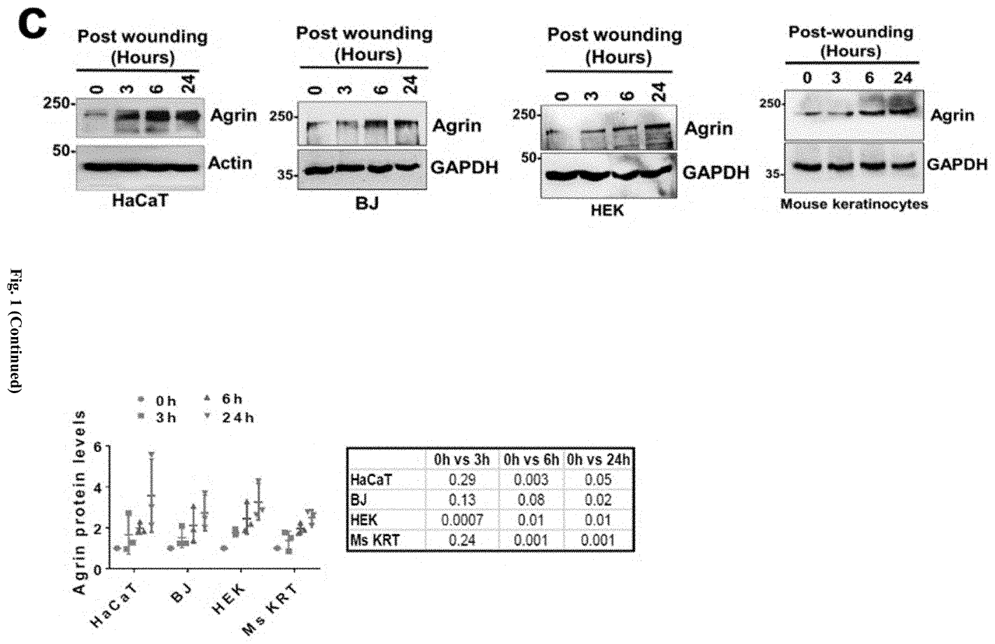

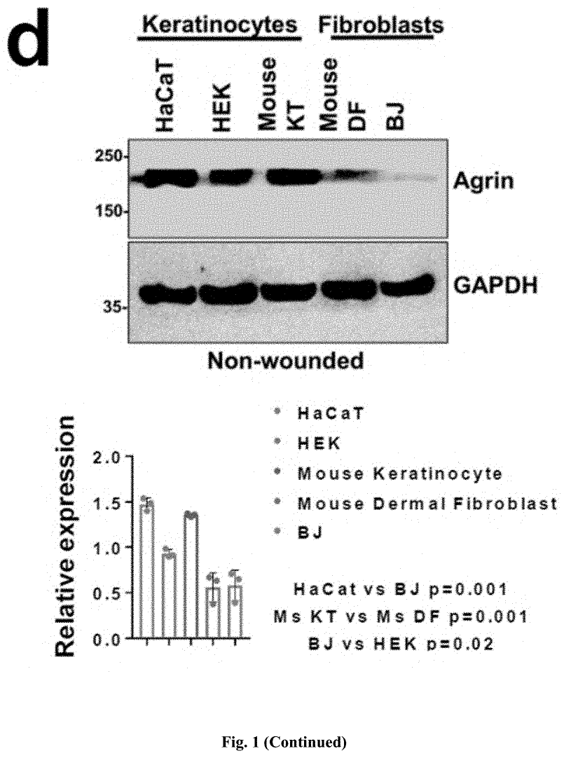

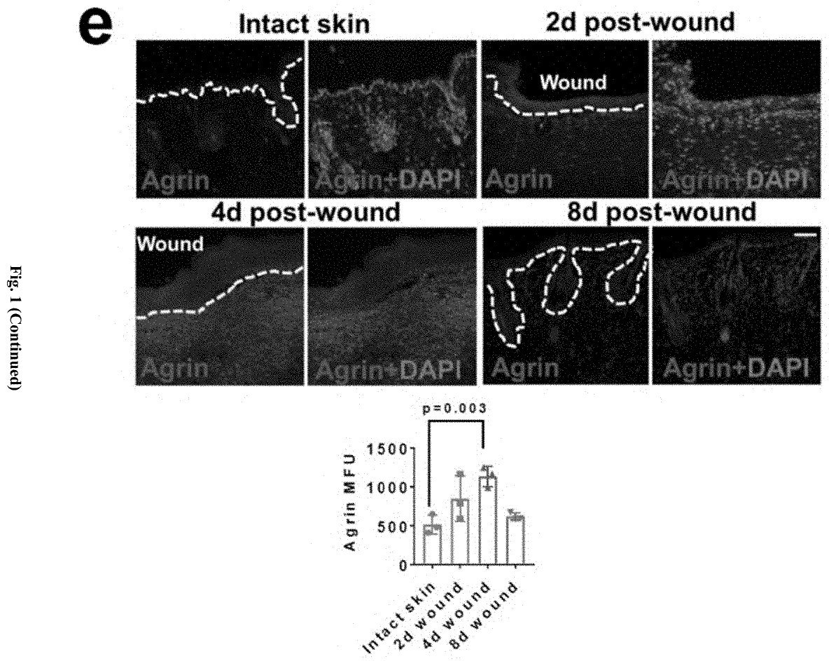

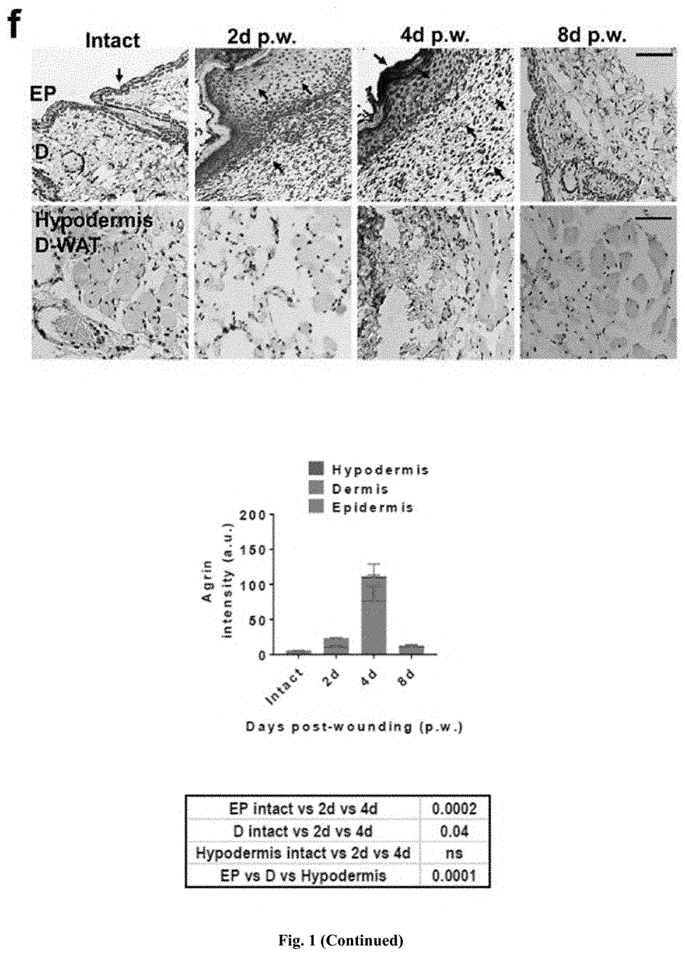

Epithelial wound healing is dramatically influenced by the re-establishment of lost ECM components to generate a new stroma that supports re-epithelialization of keratinocytes facilitating wound closure. Focusing on the early phase of the wound healing where re-epithelization is marked by Keratin 17 (KRT17) expressing keratinocytes, we identified a wound signature of ECM proteins including Agrin (AGRN), Perlecan (HSPG2), Glypican 1-3 (GPC1-3), that were enhanced within days 1-10 post punch wound biopsies in mouse skin (FIG. 1a). Following this trend, the expression levels of these ECM proteins were validated in vitro using a scratch wound model that phenocopies a mechanically challenged environment in several skin cell types including the immortalized and primary human keratinocytes (HaCaT and HEK) and human skin fibroblasts (BJ), respectively. As shown in FIG. 1b, the inventors analyzed the mRNA and protein levels in the migratory cells that initiate wound coverage within an early time-frame lasting for 24 hours. Proteoglycans AGRN, GPC1, and HSPG2 were chosen as they were significantly upregulated during the early phase of healing in mice models (FIG. 1a). An increase in the mRNA levels of the selected ECM wound signature gene(s) was observed across the panel of skin cells that initiated migration following mechanical wounding; out of which the expression of AGRN consistently increased in all the analyzed skin cell types during the observed 24 hours post-wounding (FIG. 1b). Of note, the Agrin expression significantly increased in the keratinocytes HaCaT and HEK within 3-24 h post-injury, whereas significant enhancement in fibroblasts (BJ) were observed by 24 h post-wounding (FIG. 1b). In contrast, GPC1 mRNA increased by 2-5 folds only in HaCaT cells within 3-24 h post-injury without showing significant increment in HEK and BJ cells (FIG. 1b). HSPG2 did not show any significant increase in any of the analyzed cell lines (FIG. 1b). Consistent with the data analyzed in FIG. 1a, it was observed that depleting GPC1 displayed greater inhibition of HaCaT cell migration velocities post-wounding when compared to that of Agrin knockdown (FIG. 2a-c). Despite the fact that Agrin expression was induced more robustly in both keratinocytes and fibroblasts upon injury (FIG. 1b), GPC1 may also serve as an important ECM proteoglycan promoting skin wound healing. In this study, the inventors focused on Agrin as its role has never been documented in skin injury-related models. Besides, the protein levels of Agrin also showed a consistent ˜2-4 folds increase within the observed 24 h post-wounding across the panel of human and mouse keratinocytes and fibroblasts (FIG. 1c). Even in non-wounded states, both human and mouse keratinocytes expressed higher Agrin levels when compared to their respective dermal fibroblasts, implying that keratinocytes sourced Agrin may play critical roles in skin wound repair (FIG. 1d). In a 4 mm punch-wound healing mouse model, robust Agrin expression was detected around wound edges and in wound-beds as early as day 2, which peaked at day 4 and subsequently normalized within day 8 post-wounding (FIG. 1e). Immunohistochemical analysis further revealed a significant surge of Agrin expression within the keratinocyte layers of the epidermis in comparison to the dermis at day 2 that maximized by day 4 post-wound injury (FIG. 1f). While increased Agrin expression was also observed within the injured dermis layers between days 2-4, no significant change was detected in the hypodermis and dermal-white adipose tissues (D-WAT) at any stage post-injury (FIG. 1f). Together, these results revealed that Agrin expression is significantly triggered within the epidermal and dermal layers of skin upon mechanical injury.

Depletion of Agrin Impairs Skin Wound Healing

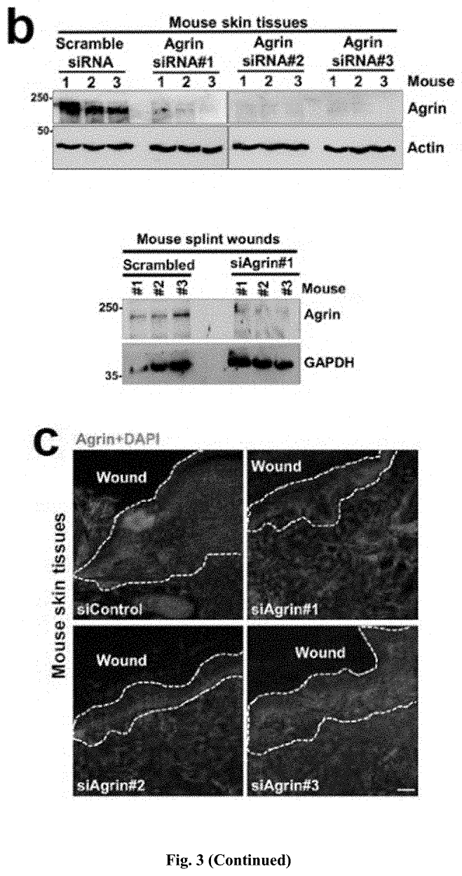

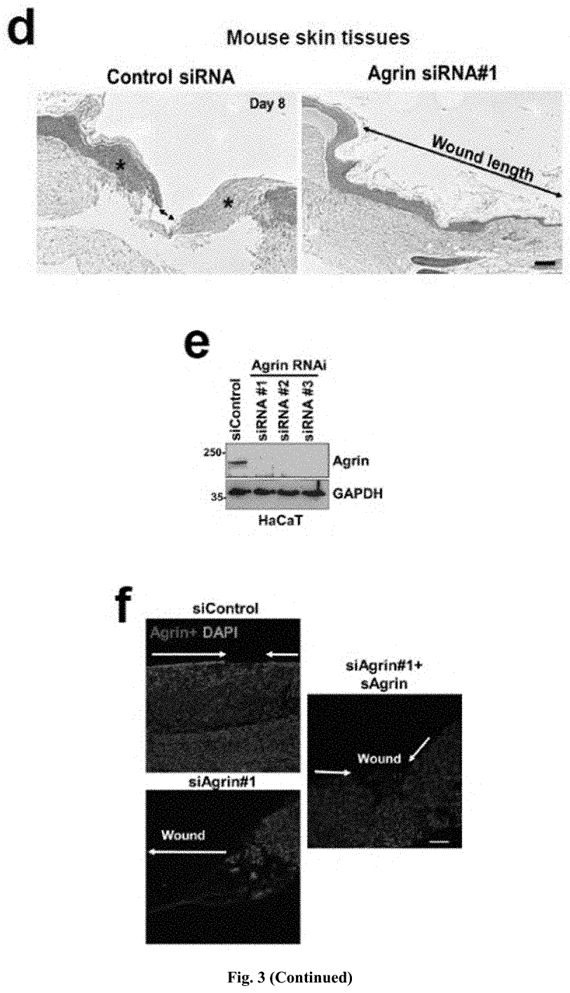

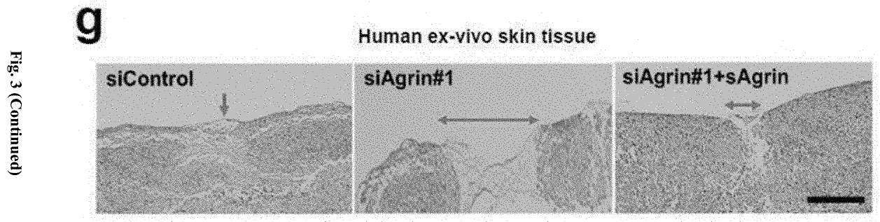

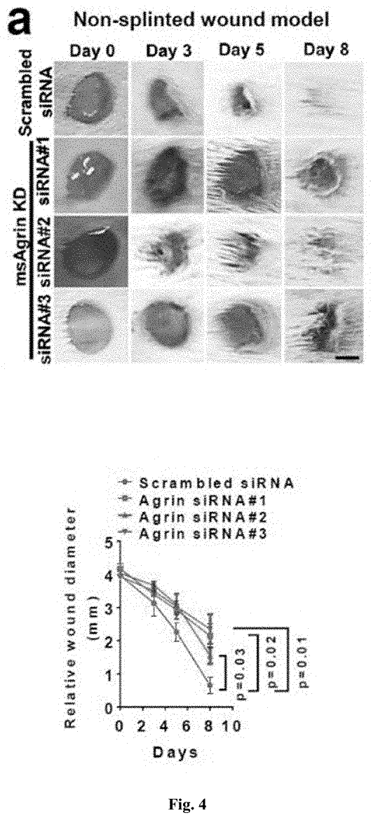

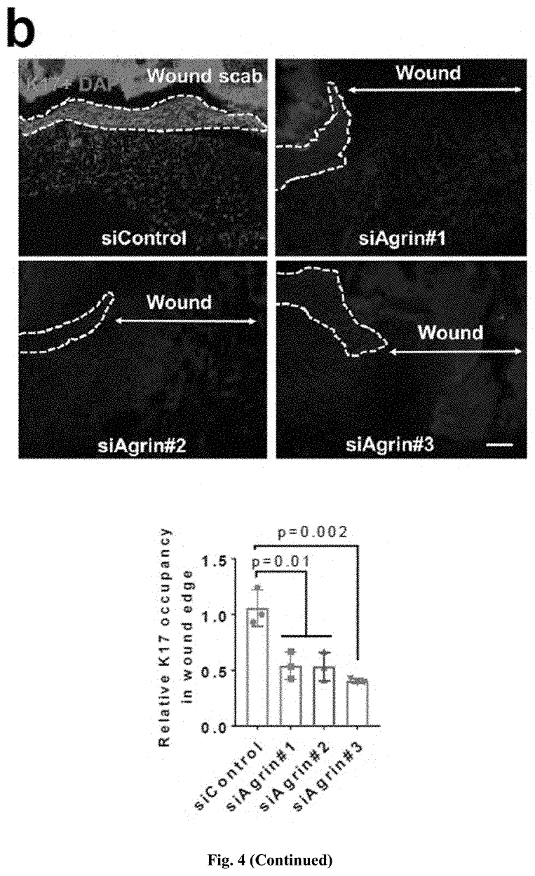

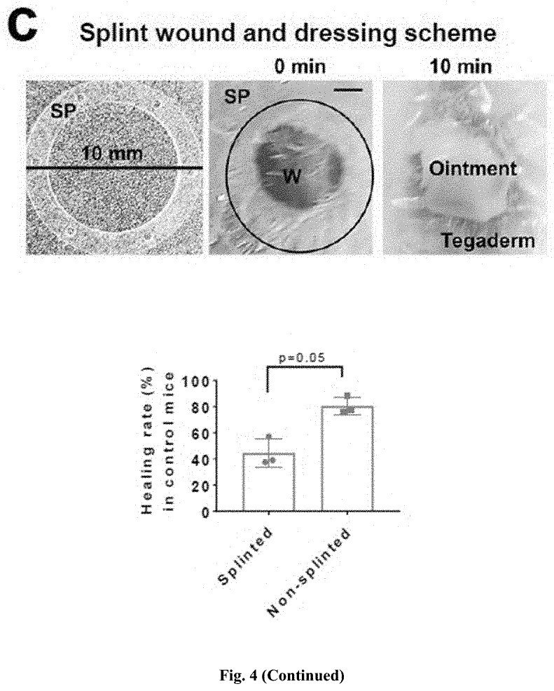

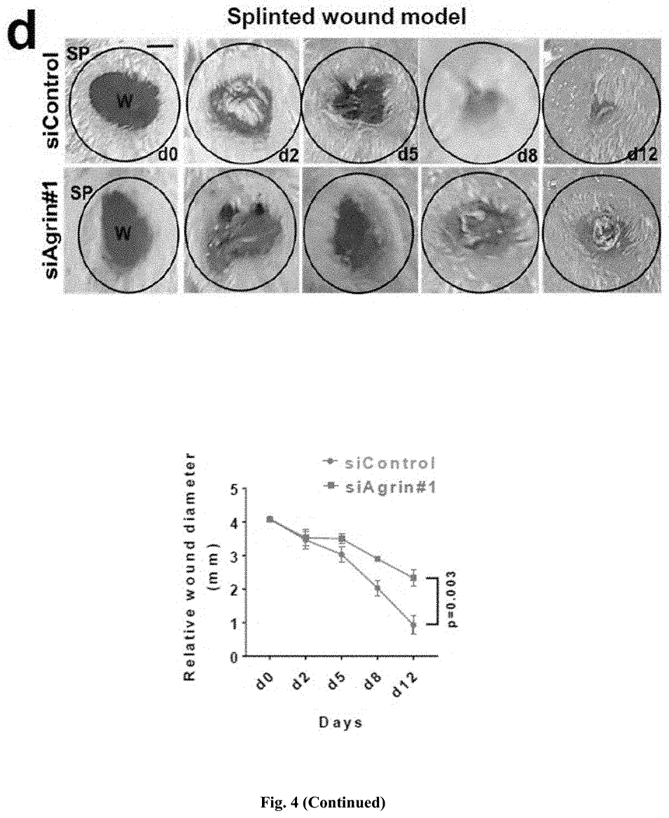

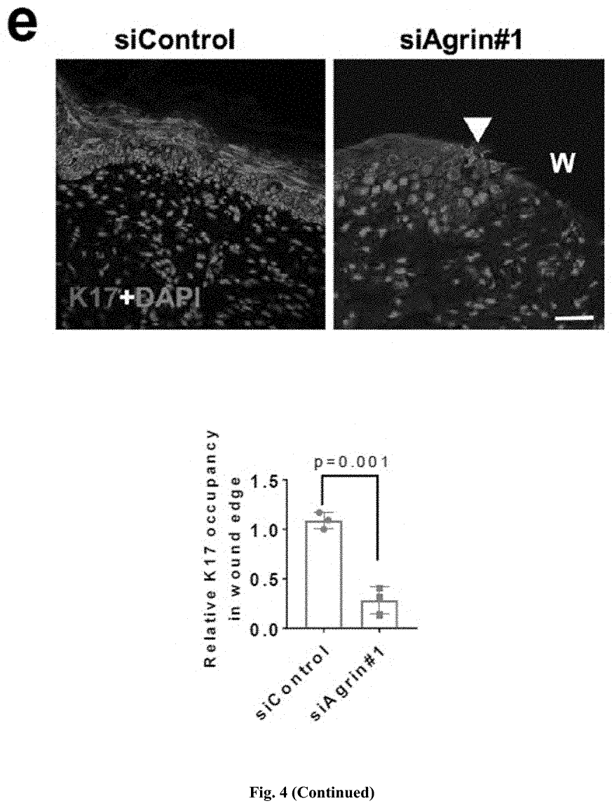

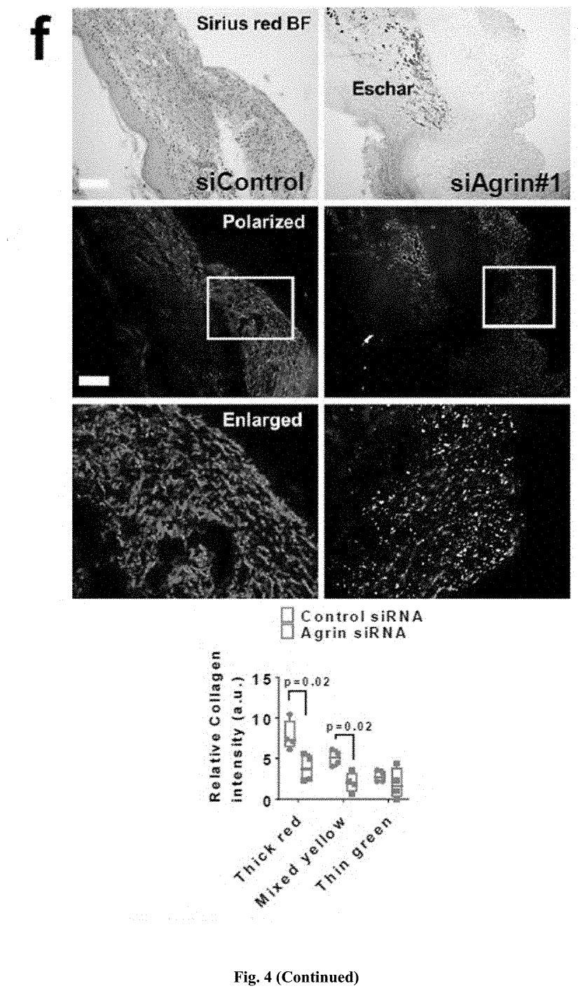

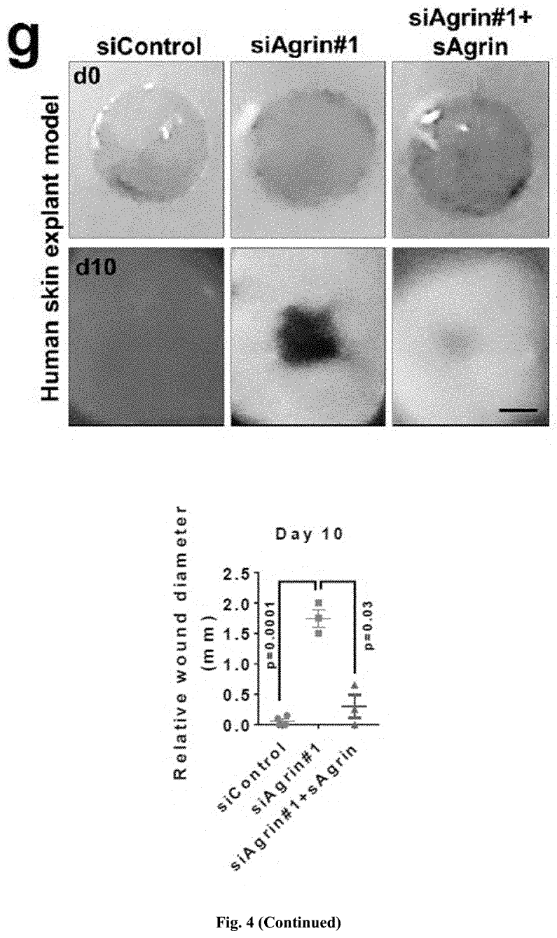

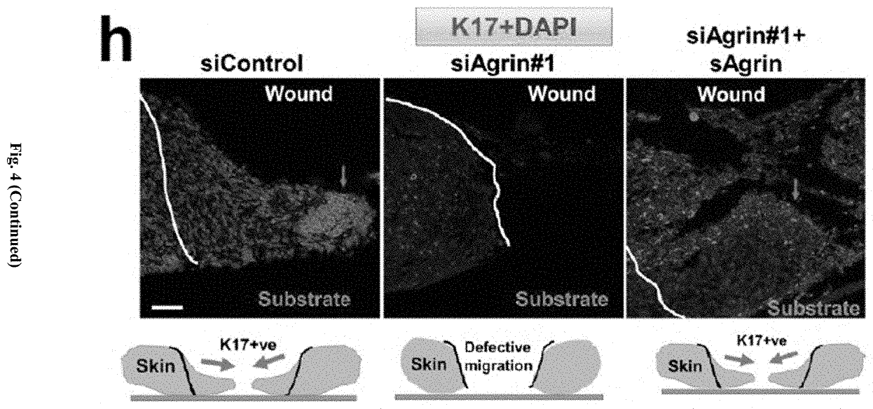

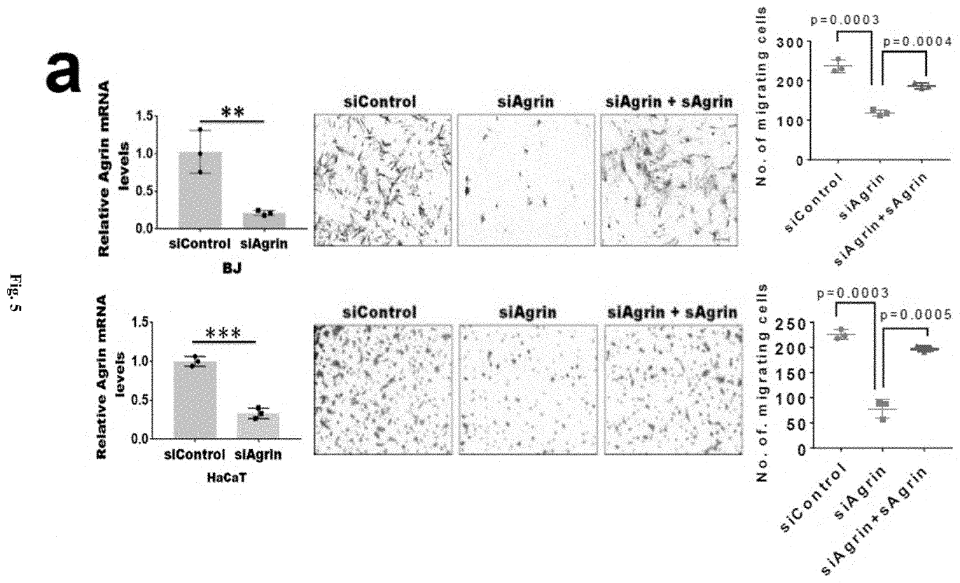

To test the functional relevance of an Agrin-enriched microenvironment in promoting wound healing in vivo, the inventors utilized three independent stealth siRNAs to knockdown Agrin in the mouse skin to see the impact on healing rates following punch-biopsy wounds under ‘non-splinted’ and ‘splinted’ conditions, respectively (FIG. 3a). Stealth siRNAs offer enhanced stability, minimal off-target effects, and accessibility to skin tissues, hence are increasingly used for efficient knockdowns in animal models. In both models, the siRNAs were locally injected at the prospective wound site three days before wounding that efficiently reduced the basal Agrin levels at the skin injury site on the day of wounding (FIG. 3b, left panel). The scrambled or Agrin siRNAs were mixed in a topical ointment preparation and applied at the open wound site every two days to robustly deplete Agrin expression throughout the early phase(s) of healing comprising of 9 days post-wounding in the non-splinted models. At day 4, strong Agrin expression detected around wound edges and wound bed of control skin was strikingly diminished in mice ectopically treated with Agrin siRNAs which validated the efficacy of Agrin knockdown (FIG. 3c). The suppression of Agrin expression significantly delayed the in vivo cutaneous wound healing, indicating that Agrin is important for skin wound repair (FIG. 4a). Moreover, efficient wound healing is dependent on the ability of keratinocytes to express several wound responsive Keratins such as Keratin 6, 14, and 17, that propel the formers' migratory behavior over the wound site. Wound closure is initiated by a stretch of migrating keratinocytes referred to as ‘epithelial tongue’. Prominent epithelial tongue predominantly concealed the wound site in scrambled siRNA treated skins while leaving the vast majority of wound uncovered in Agrin depleted skin sections (FIG. 3d). In this vein, a significant loss (>50%) of keratinocyte migration (as shown by the relative K17 occupancy) over the wounded site was observed in mouse skin treated with Agrin siRNAs (FIG. 4b).

Furthermore, the 4 mm punch-wounds were surrounded by a 10 mm splint tightly adhered to the skin, thereby representing a ‘closed’ splinted condition for wound healing. The ointments containing the respective siRNAs were applied and the wound region were subsequently covered by Tegaderm (FIG. 4c). As such, the usage of splints minimized the ‘purse-string’ mediated wound contraction and majorly facilitated wound closure by re-epithelialization. Covered splinted wound dressings reduced wound healing rates at day 7 when compared to those in non-splinted conditions (FIG. 4c). Using this in vivo model as an index for measuring keratinocytes' migration, cutaneous Agrin levels were efficiently suppressed (FIG. 3b, right panels). Agrin depletion in splinted mouse models delayed skin wound healing by attenuating keratinocyte re-epithelialization as shown by reduced K17 occupancy (FIGS. 4d-e). Coupled to impaired re-epithelialization, Agrin depletion severely dampened the deposition of mature and intermediate collagen fibers in the wound beds, indicating that compromised ECM replenishment in Agrin depleted skin resulted in delayed healing response (FIG. 4f).

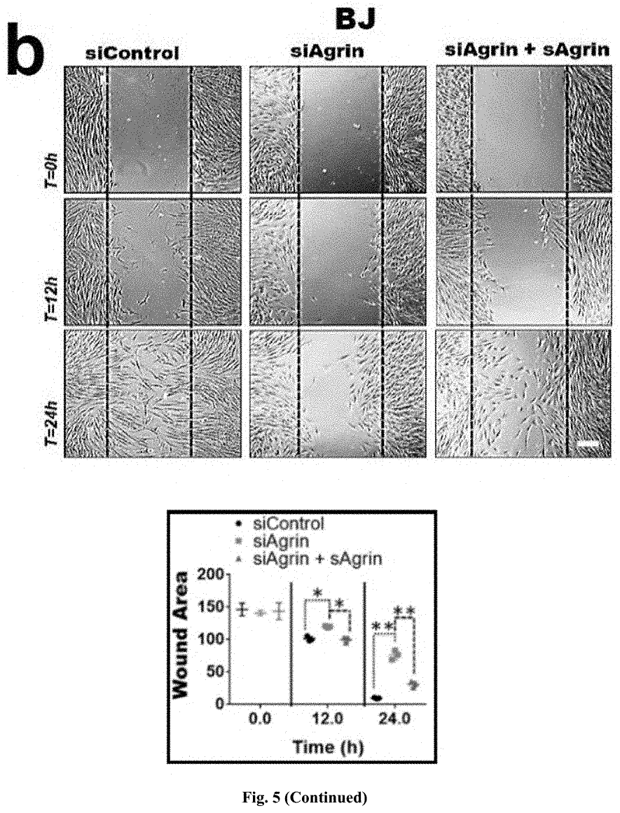

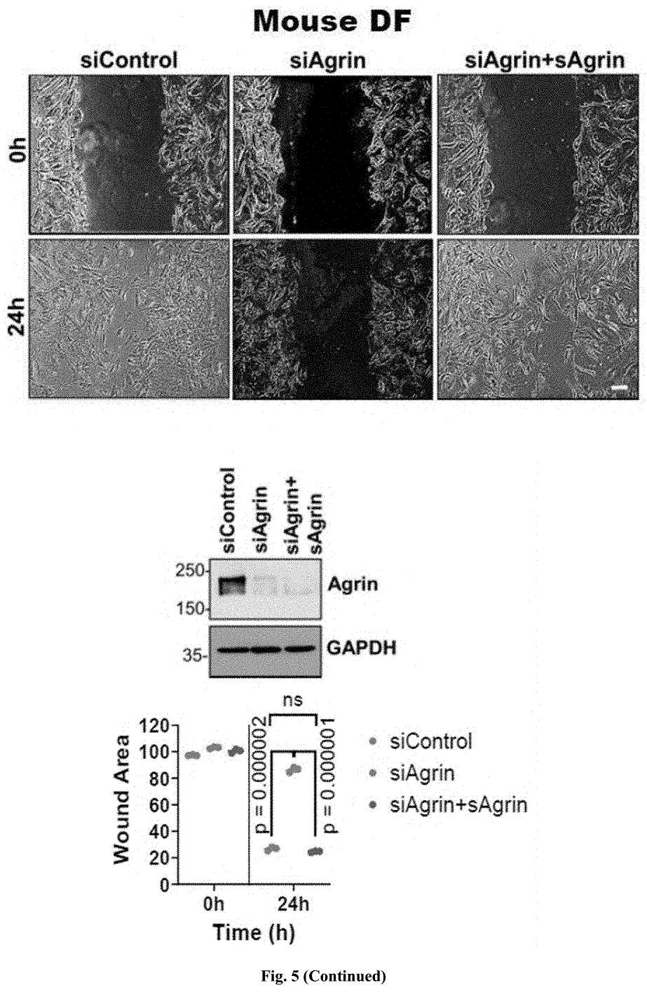

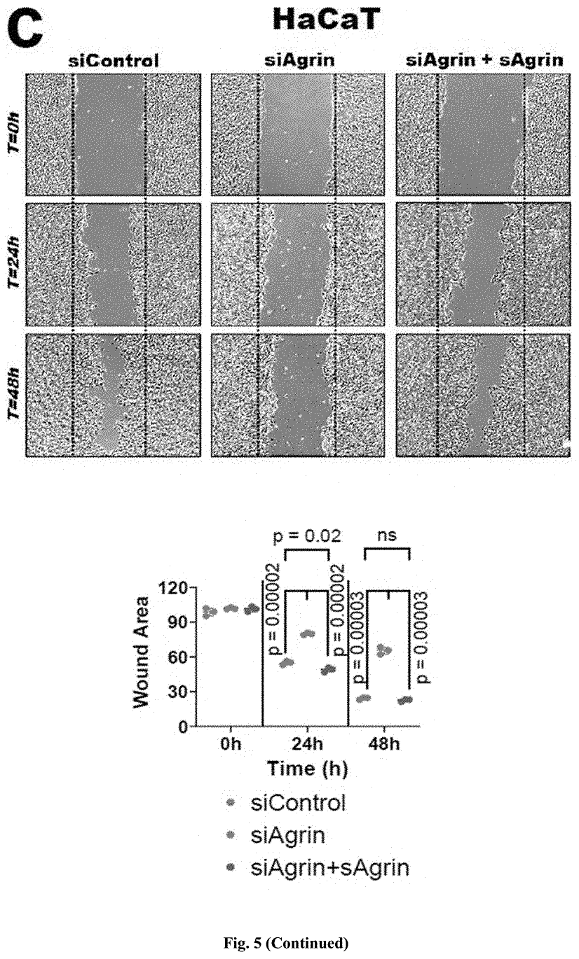

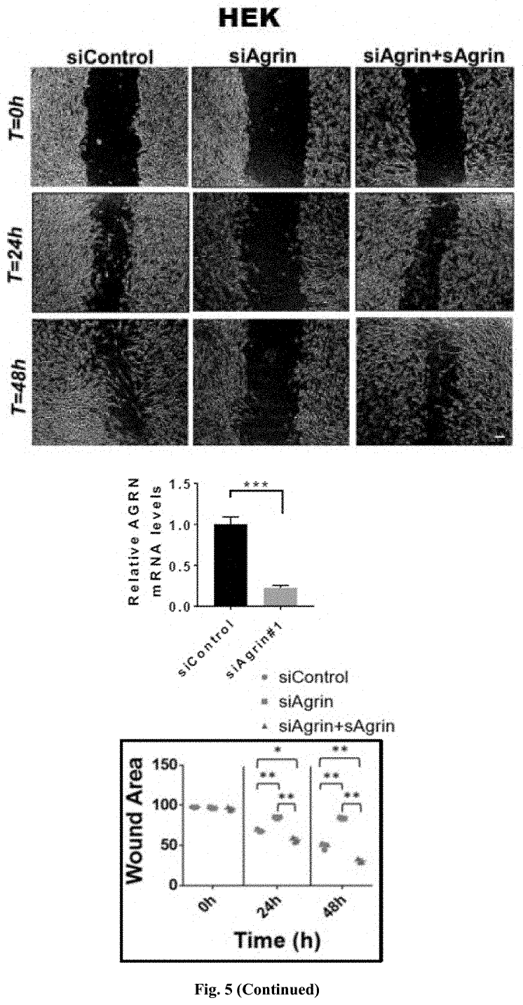

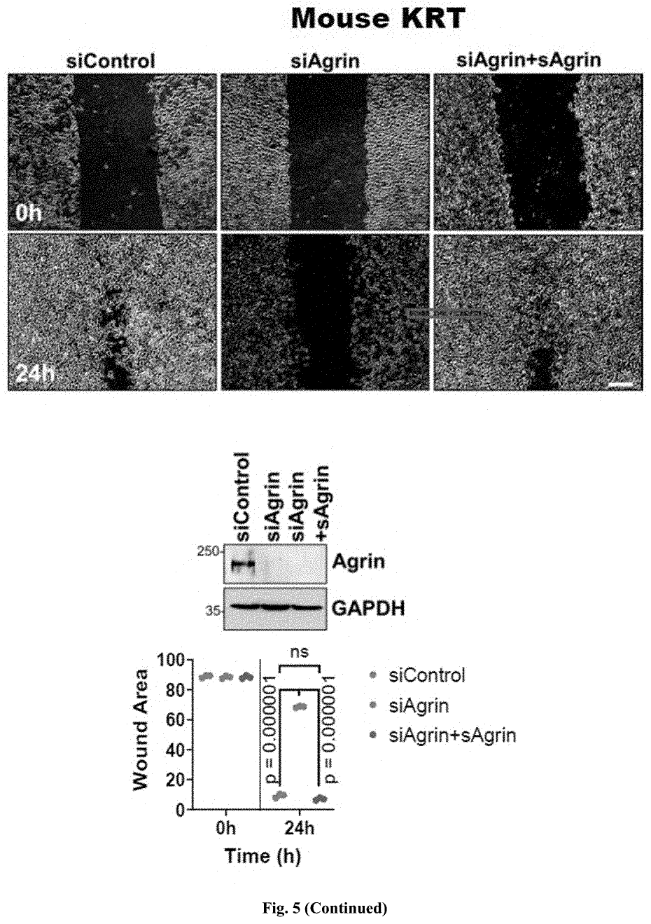

Moreover, siRNAs against human Agrin were used to inhibit its expression in keratinocyte and in a human skin explant model (FIGS. 3e-f). Notably, Agrin expressing keratinocytes effectively migrated to close the wound in control siRNA treated skin explants, which was drastically inhibited by Agrin siRNA #1 (FIGS. 3f-g). The depletion of Agrin (by Agrin siRNA #1) robustly attenuated the K17 expressing epithelial tongue migration and thereby significantly retarded the ex vivo wound closure rates in these human skin explants (FIGS. 4g-h, 3g). Interestingly, supplementing the C-terminus recombinant protein fragment of Agrin (sAgrin) harboring the binding sites to its receptors Lipoprotein related receptor-4 (Lrp4) and integrins, significantly rescued the wound healing and the migration of keratinocytes in the human skin explant model (FIGS. 4g-h). In accordance to these in vivo and ex vivo functions, knockdown of Agrin severely impaired the trans-well migration and scratch wound-responsive migration velocities in dermal fibroblasts and keratinocytes (of human and mouse origin) that was again in part, rescued by sAgrin (FIGS. 5a-d).

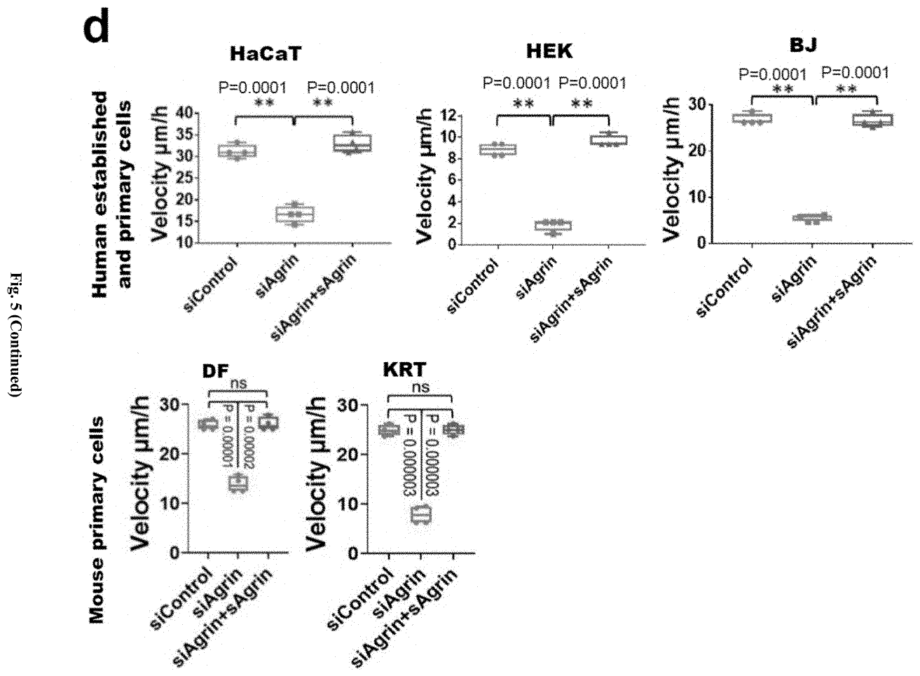

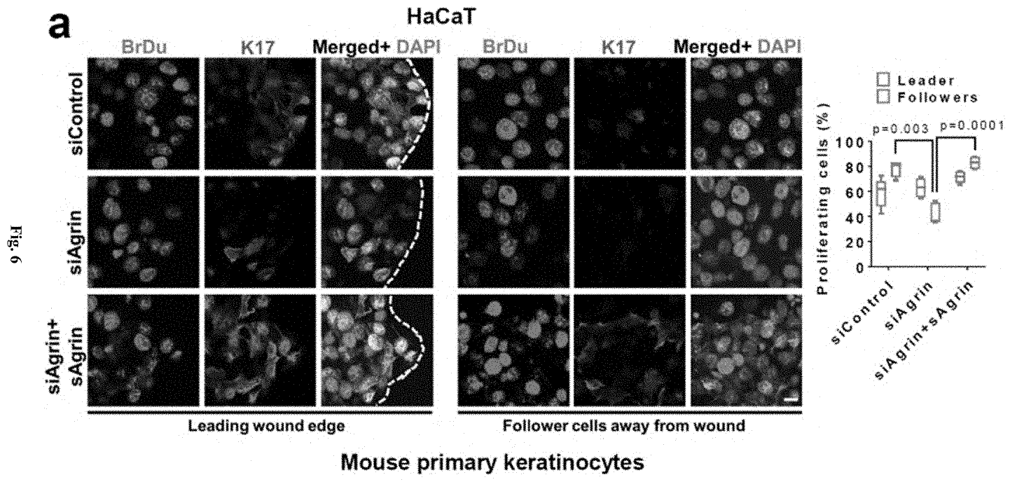

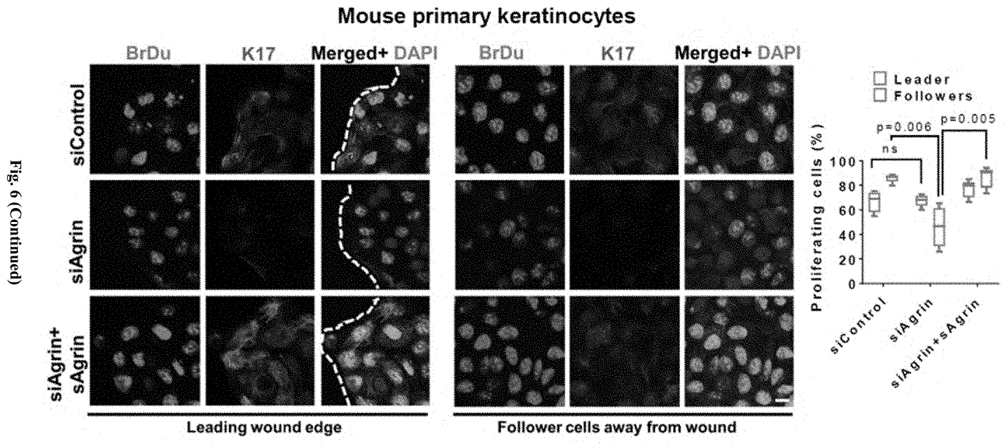

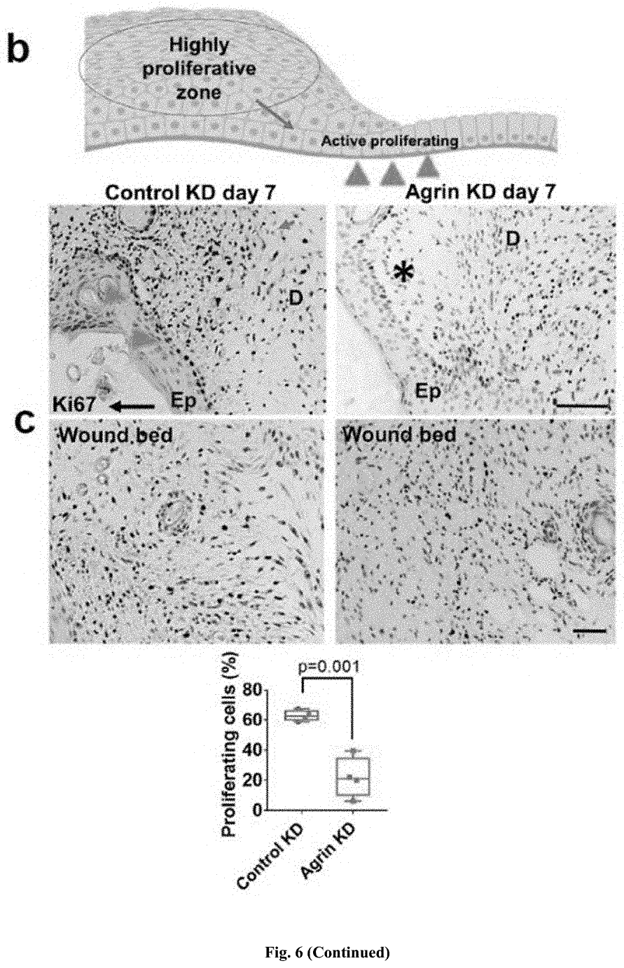

As keratinocyte re-epithelialization is tightly coordinated with their proliferative states, it was next documented whether Agrin supported keratinocytes' proliferation during their migratory phase post-wound injury. During early in vitro migration within 4 h post-wound injury, Agrin depletion did not affect the proliferative rates of the leader cells as measured by 5-bromo-2′-deoxyuridine (BrdU) incorporation assays (FIG. 6a). However, the proliferation of follower cells away from the wound margin were significantly reduced by Agrin knockdown, an effect which was rescued by sAgrin supplementation (FIG. 6a). Thus, Agrin depletion differentially affects the proliferative states of leader and follower cells in vitro. In vivo, an efficient wound re-epithelialization is achieved when highly proliferative keratinocytes (followers) at the wound edge push the migrating cells (leaders) over the wound (FIG. 6b). As shown in the control siRNA treated wound sections, proliferative keratinocytes at the base of the epidermis (filled arrows) are actively pushed during re-epithelialization by the hyper-proliferative wound edge regions (arrows) at day 7 (FIG. 6b). Coupled to impaired proliferation at the base of epidermis, Agrin depletion severely hampered the keratinocyte proliferation in the dermal layers at the wound edge and beds, respectively (FIGS. 6b-c). Therefore, these shreds of evidence suggest that Agrin enriched in the wound environment supports keratinocyte proliferation that primes an efficient collective migration phase and wound closure.

Agrin Sensitizes Keratinocytes Towards ECM Rigidity and Fluidic Collective Migration

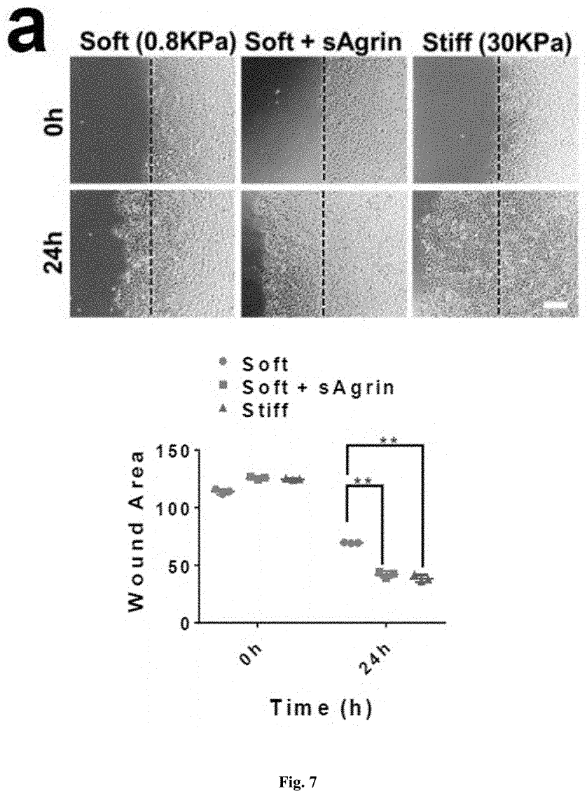

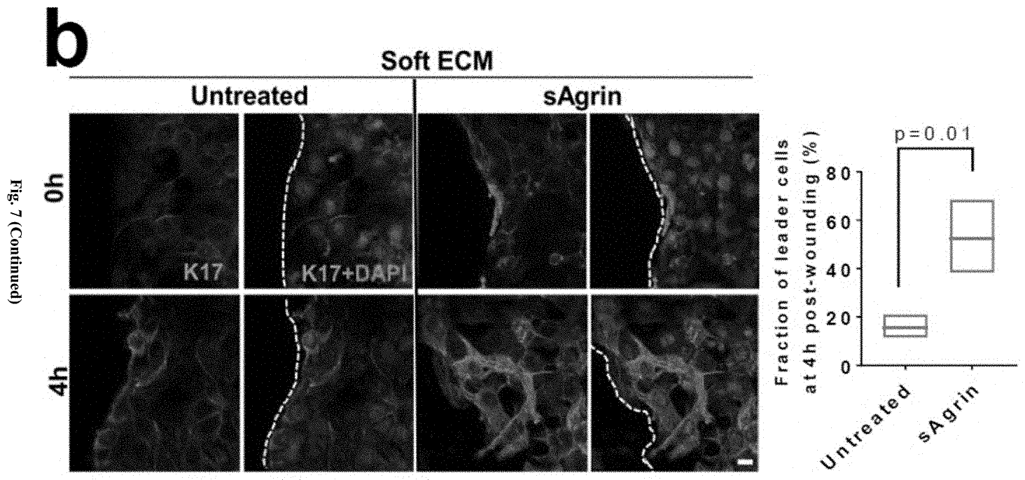

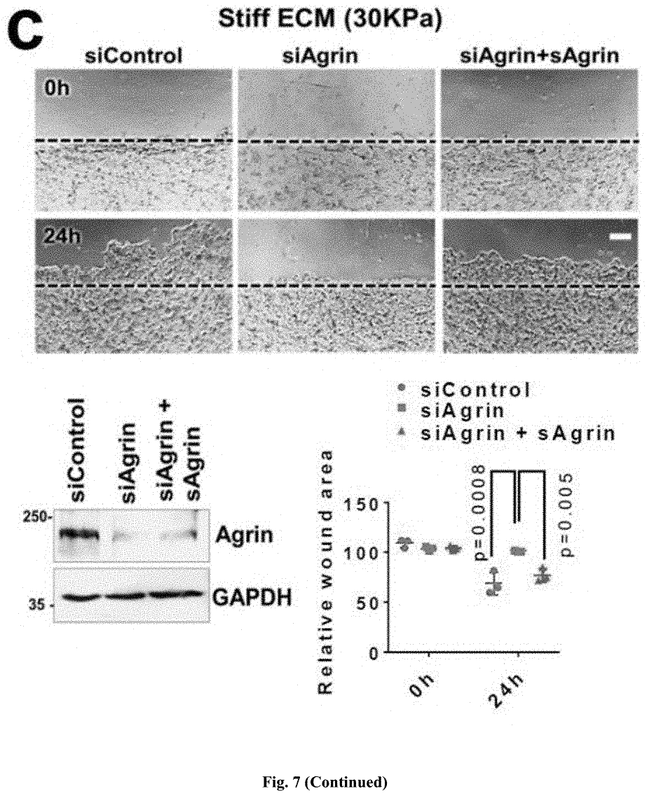

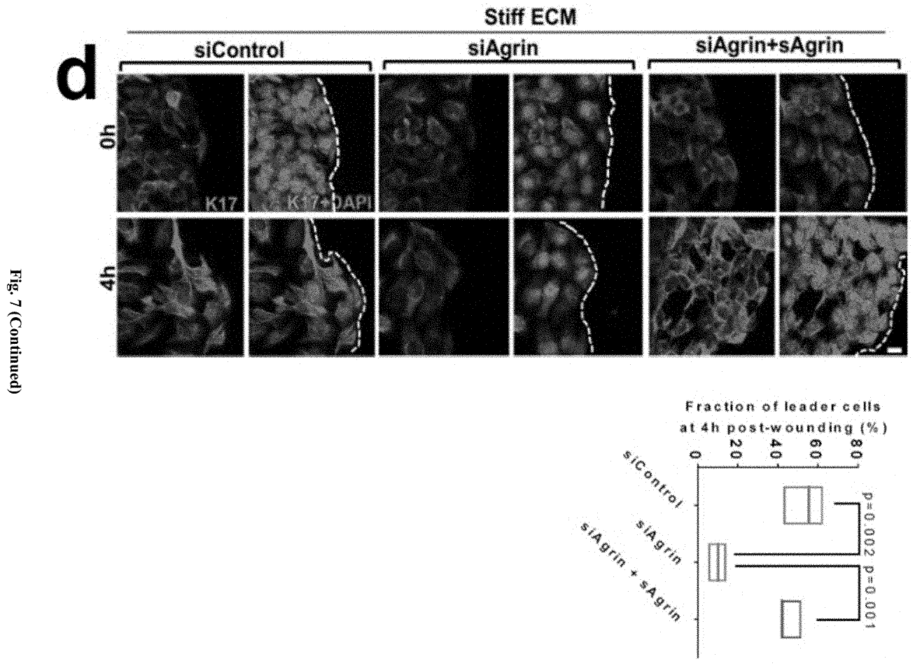

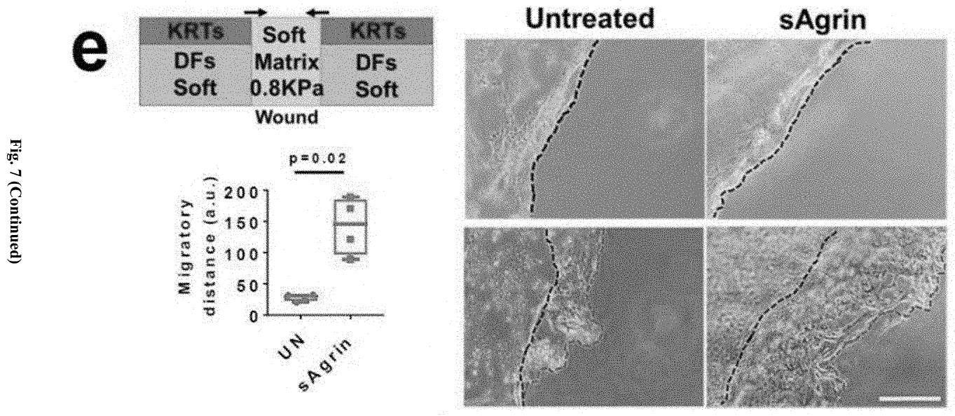

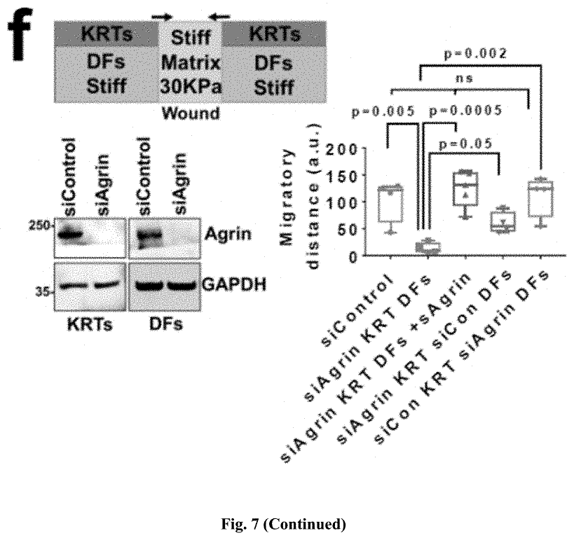

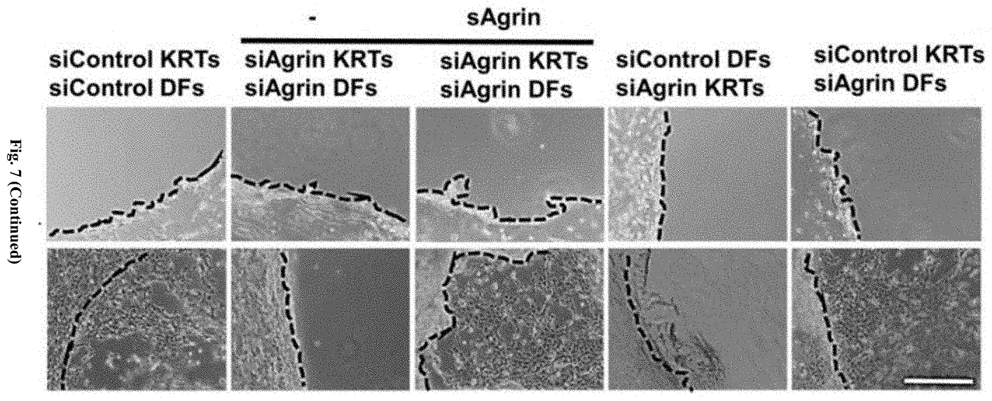

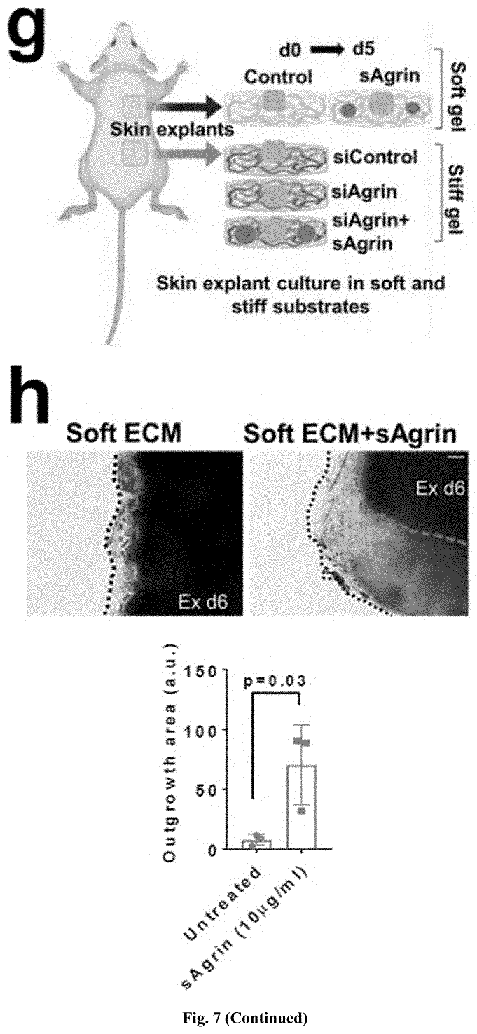

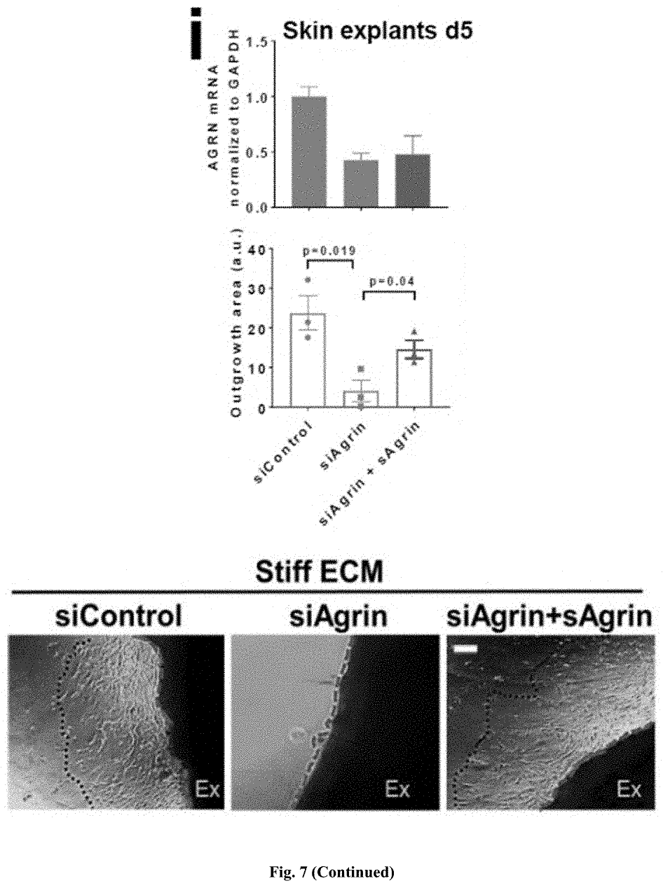

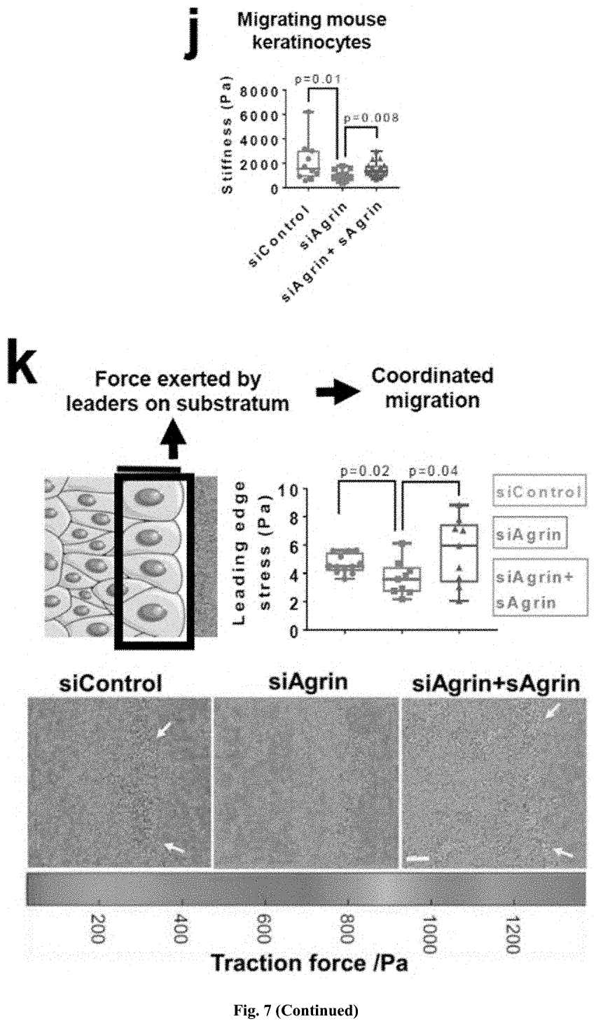

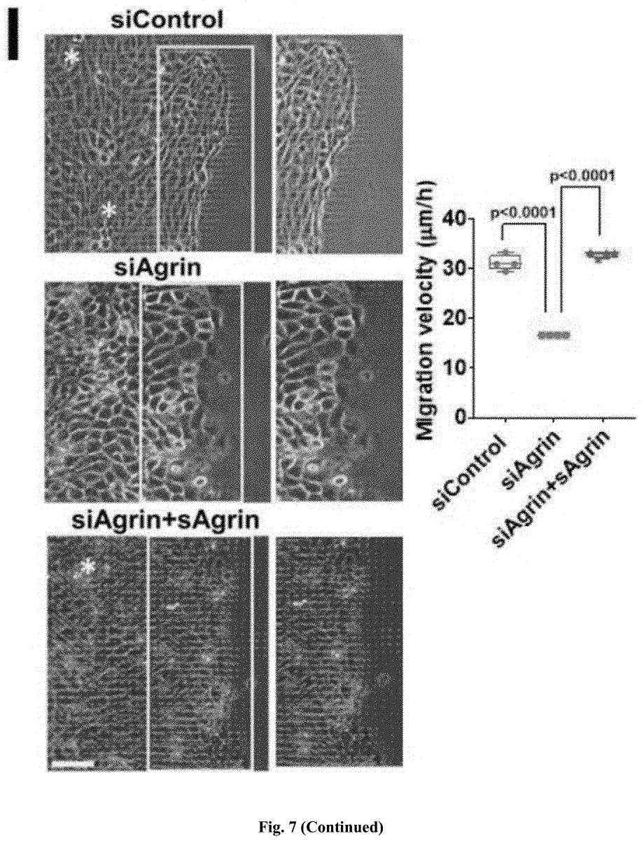

Having demonstrated a role in wound healing response, the inventors explored whether Agrin generates a mechanically competent environment favoring collective keratinocyte migration and wound closure. Since bulk stiffness from the ECM stimulates the migration in a variety of cell types, the inventors rationalized that Agrin may integrate ECM stiffness signals and collective keratinocyte migration within the wounded skin environment. Collective migration of HaCaT cells cultured on stiff (30 kPa) substrates was significantly higher than in compliant ones (0.8 kPa) (FIG. 7a). Quite interestingly, incorporation of sAgrin within compliant substrates significantly promoted migration rates that was comparable to those in stiff ECM alone (FIG. 7a). The collective keratinocyte migration is initiated by the expression of Keratin 17 (K17), particularly by the leader cells located at the wound edges. In such conditions, supplementing sAgrin to soft substrates stimulated collective migration by activating K17 expressing leader cells (FIG. 7b). In contrast, compared to control cells, Agrin depleted keratinocytes on stiff substrates had slower migratory potential and this was significantly rescued when sAgrin was integrated into the stiff matrix (FIG. 7c). Agrin depletion in cells experiencing stiff substrates also resulted in a dramatic loss of migrating leader cells expressing K17 (FIG. 7d). The fraction of K17 expressing leader cells was significantly restored in Agrin depleted cells that sensed sAgrin supplemented in the stiff ECM and subsequently showcased enhanced migration (FIGS. 7c-d).

Complementing the 2D scratch-wound assays, the inventors next devised an in vitro 3D-substrate stiffness dependent migration assay to recapitulate the role of Agrin in mediating keratinocytes' migration under the simultaneous influence of underlying dermal cultures and bulk substrate rigidity as experienced by native skin tissues. In this strategy, primary mouse dermal fibroblasts (DFs) were first embedded within a compliant collagen matrix (0.8 KPa) which were subsequently overlaid with mouse keratinocytes (KRTs) (FIG. 7e). A wound was created at the center and the resulting area was replenished by soft matrix immediately allowing the keratinocytes to migrate through a compliant environment. As shown in FIG. 7e, supplementing sAgrin within the soft matrix strongly enhanced keratinocyte migration by day 5 post-injury. On scenarios recreated by stiff matrix, Agrin depletion in both KRTs and DFs robustly abolished the migratory rates (FIG. 7f, second panel). Interestingly, supplementing sAgrin in the stiff matrix strongly restored keratinocyte migration (FIG. 7f, third panel). Of note, Agrin expressed by the DFs was not sufficient to rescue the migration of Agrin depleted keratinocytes over the stiff substrates (FIG. 7f, fourth panel). Furthermore, Agrin depleted DFs had minimal effects on blocking the keratinocyte migration over stiff substrates (FIG. 7f, fifth panel). Together, these data indicate that increased Agrin expression within keratinocytes majorly responds to ECM rigidity sensing which is necessary and sufficient to drive migration in the wound microenvironment.

In the next paradigm, the ability of sAgrin to sensitize collective ex vivo keratinocyte outgrowth from mouse skin explants experiencing varied substrate rigidity was tested. Accordingly, collective keratinocyte outgrowth was monitored when full-thickness hind skin from 2-day old mouse pup was placed on either collagen matrix corresponding to soft or stiff substrates in the presence or absence of sAgrin, respectively (FIG. 7g). Compared to the explants on soft collagen matrices alone, sAgrin supplemented compliant substrates significantly enhanced keratinocyte outgrowth (FIG. 7h). Agrin depletion by mouse specific siRNA treatment in these skin explants experiencing a stiff substrate without any exogenous sAgrin failed to generate keratinocyte outgrowth after five days post-culture (FIG. 7i, middle panel). In distinction, Agrin depleted skin explants that were sensitized by sAgrin scaffolded stiff substrates exhibited higher keratinocyte outgrowth (FIG. 7i, third panel). Cumulatively, these data suggest that Agrin empowers ECM rigidity sensing in keratinocytes that guides collective migration post-wounding.

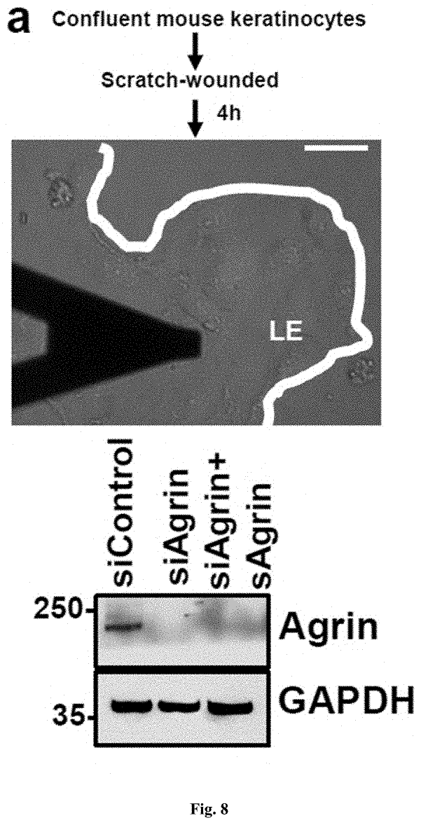

Cells respond to bulk ECM stiffness by introducing subtle changes to their intrinsic material properties by enhancing their cytoskeletal tension that favor greater motility. To examine the mechanoperception of keratinocytes bestowed by an Agrin-enriched environment, the stiffness of migrating keratinocytes post-injury was first measured by Atomic Force Microscopy (AFM) as a marker for cell-intrinsic material property. The stiffness of migrating mouse keratinocytes recorded as ˜2.1 KPa at 4 h post-wound scratch was significantly decreased to ˜0.9 KPa upon Agrin knockdown (FIGS. 7j and 8a-b). Notably, sAgrin attributed significant increase in cell stiffness (˜1.5 KPa), thereby enhancing mechanoperception of these migrating keratinocytes (FIGS. 7j and 8b). As such, Agrin knockdown ‘softens’ the migratory cell making them incompetent to navigate across wound bed. Next, traction force microscopy (TFM) was performed to measure the cumulative forces exerted by migrating cells on the substrate in a mechanically stressed environment mimicking a wound injury. In this collective migration model, the maximal traction force is exerted by the cells at the leading edge on the ECM. The collective migration of Agrin depleted cells were severely hampered illustrating lower mean traction stress when compared to the control cells (FIG. 7k). While the control cells had higher collective traction forces at the leading edges, Agrin depleted ones had significantly lesser traction stress within the leader and follower cells that accounted for lower migratory potential (FIG. 7k). The average traction stress of cells at leading edge after 6 h post-migration was ˜4.67 Pa that reduced by ˜32% upon Agrin depletion. Also, the velocity field was mapped using particle image velocimetry (PIV) to characterize the fluidic dynamics of collective cell migration implicated in epithelial wounds. Homogenous velocity distribution was observed in control cells attributed by large magnitude velocity vectors with occasional swirling motions and vortices presenting a fluid-elastic-like migratory behavior (FIG. 7l). This fluidic migration was largely disrupted by the suppression of Agrin (FIG. 7l). Strikingly, collective traction stress at the leading front, coordinated velocity fields and fluidic collective migration were significantly enhanced by supplementing sAgrin to Agrin depleted cells (FIG. 7k-l). These results illustrate that Agrin confers cell-intrinsic stiffness and enhanced traction to ECM attributing coordinated fluidic dynamics to collective keratinocyte migration.

Agrin Mechanotransduction Tunes Cell Mechanics Post-Injury

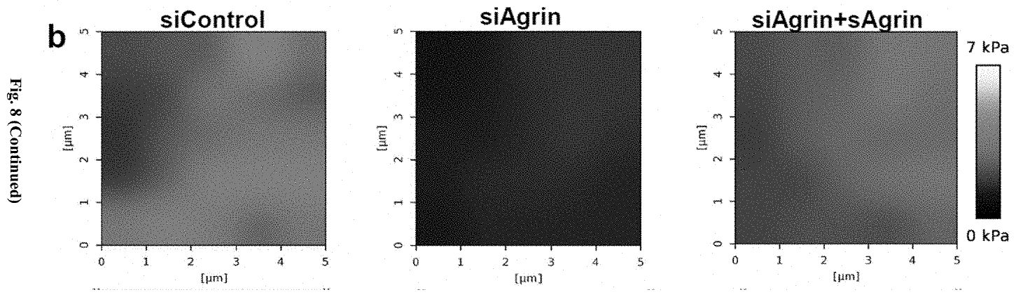

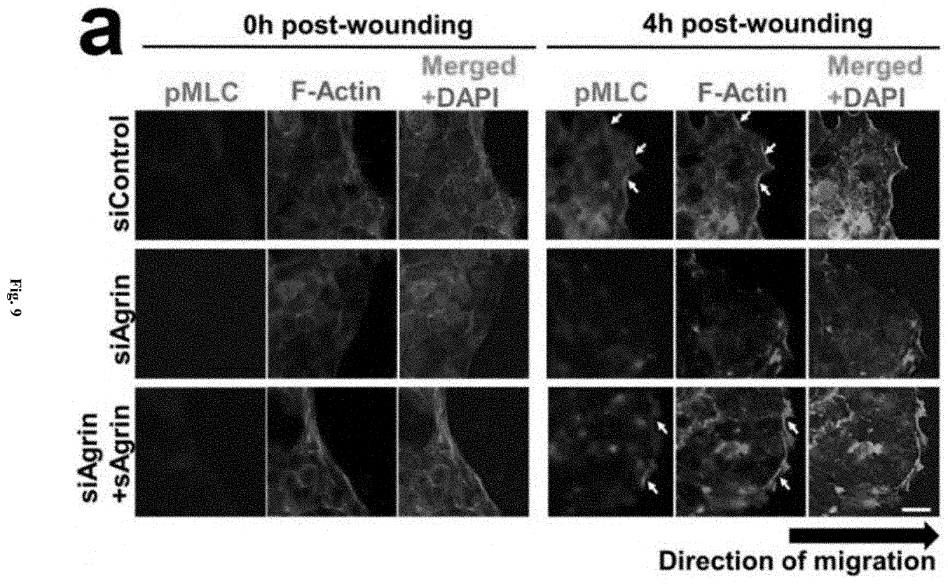

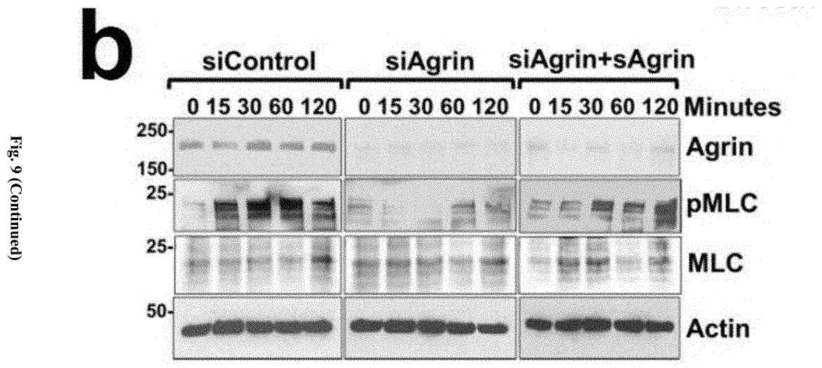

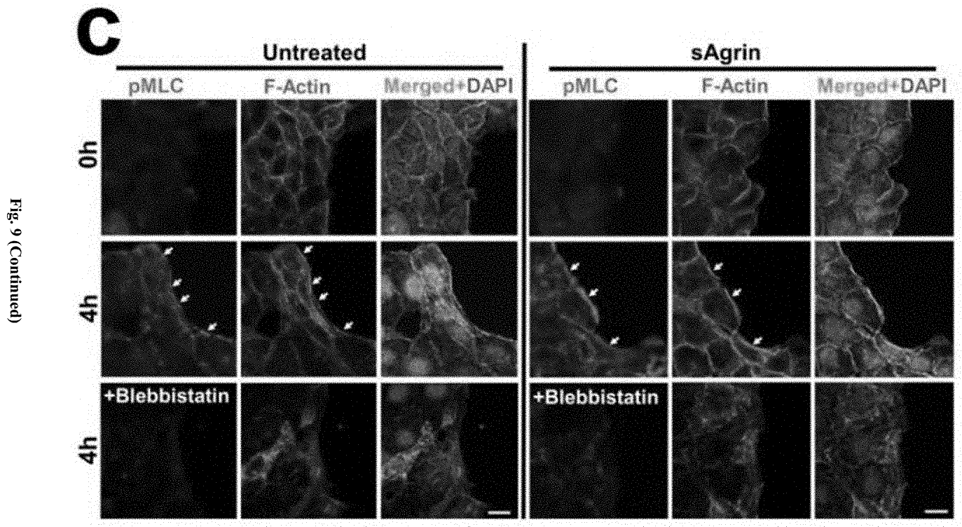

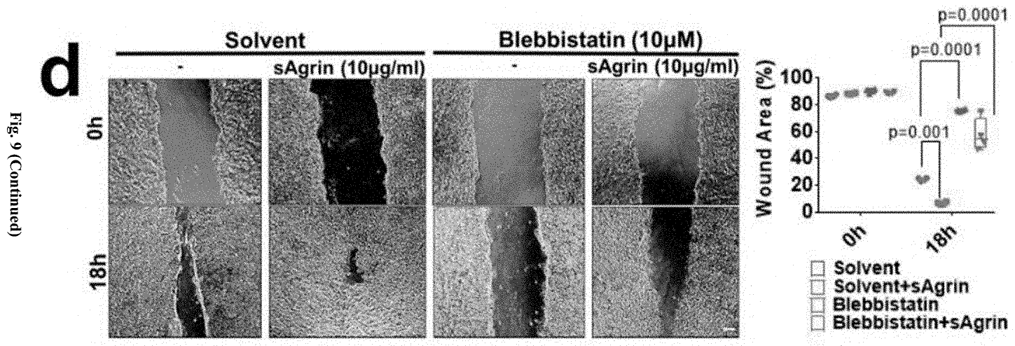

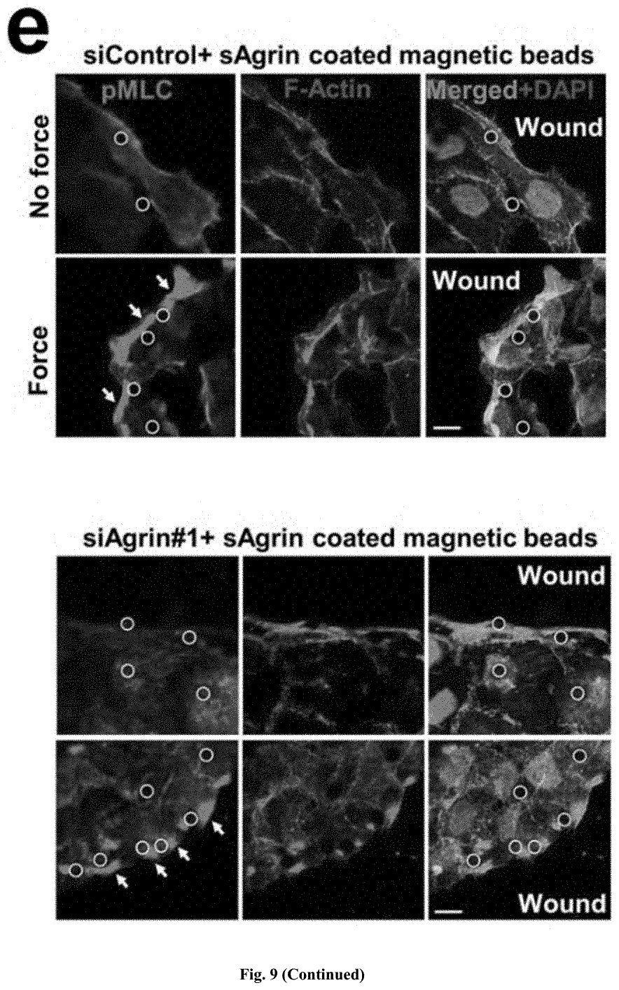

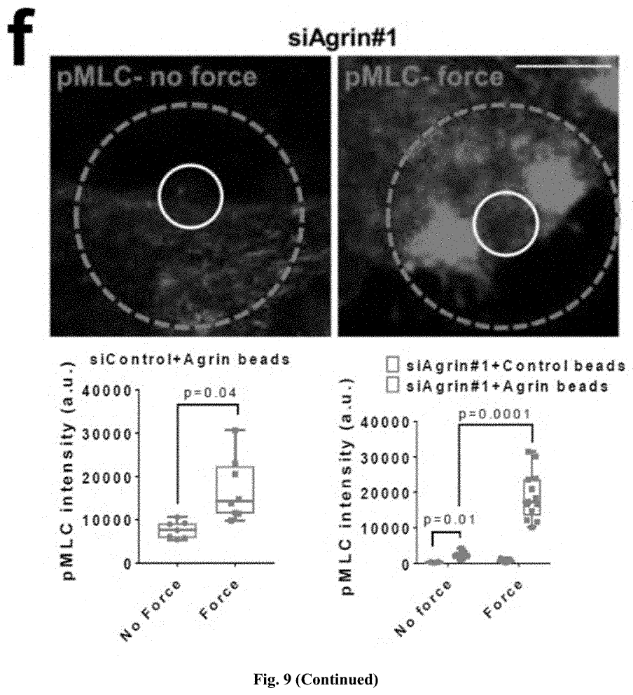

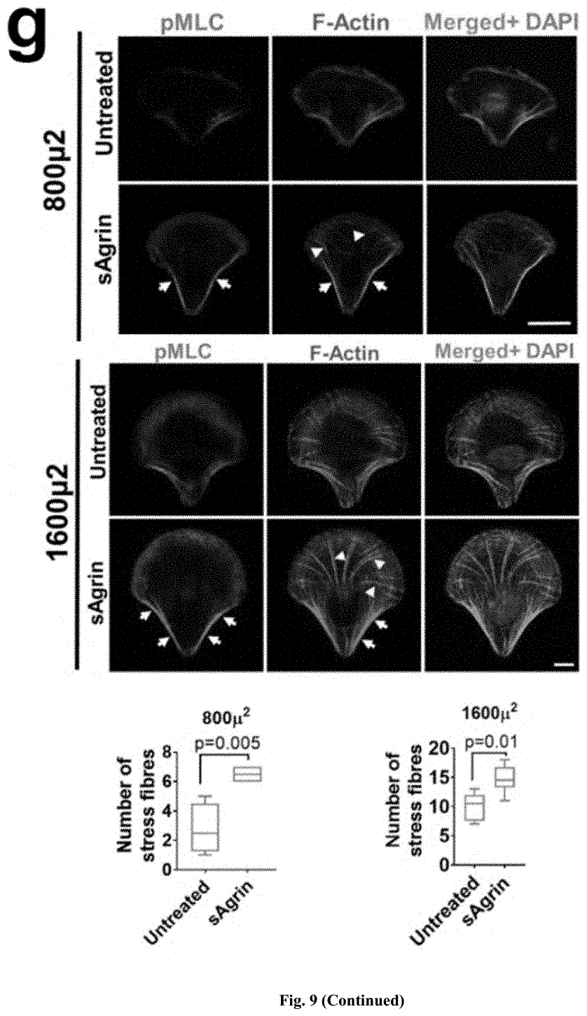

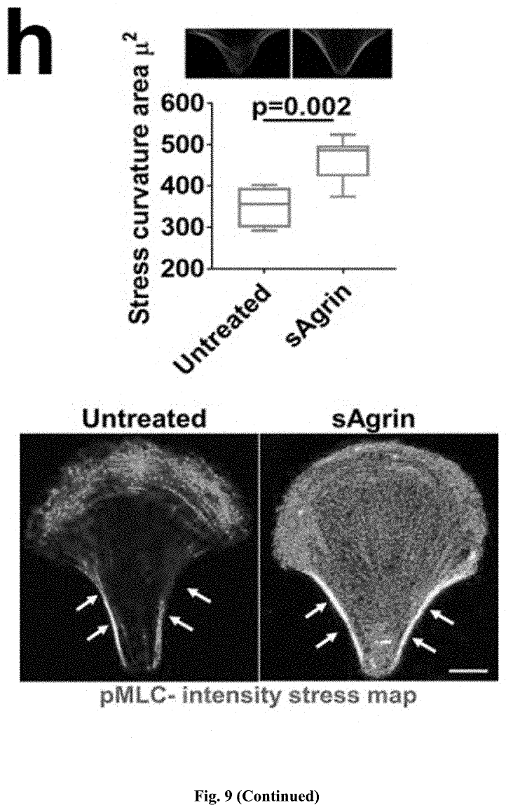

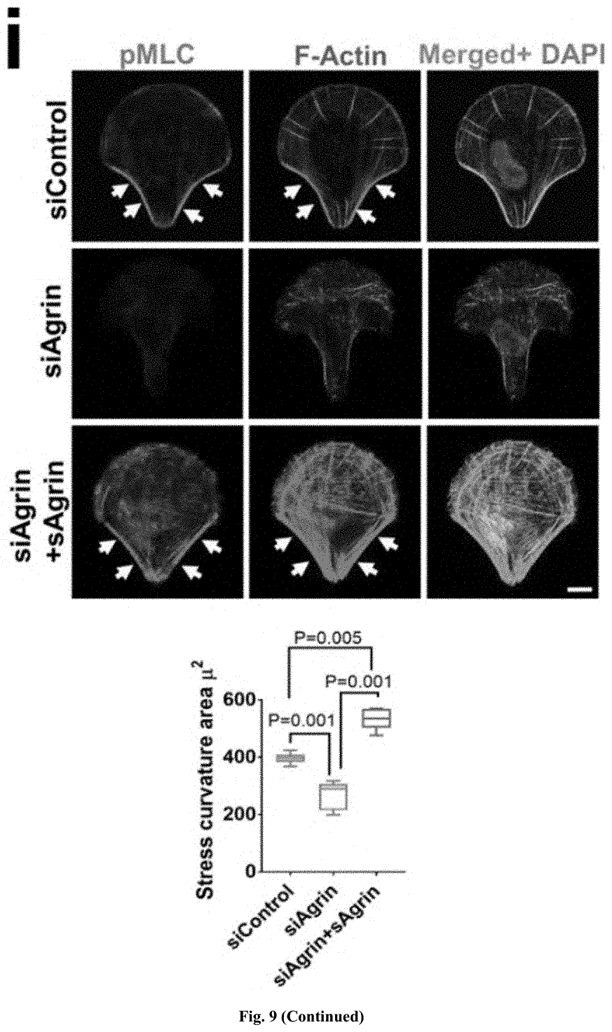

Central to the outcome of enhanced keratinocytes' fluidic motility, it was asked whether Agrin tunes cellular mechanics during wound injury via coordinating cytoskeletal architecture. An organized cytoskeletal architecture determining the integrity of collective migration in keratinocytes is showcased by the formation of actomyosin cables at the leading front in embryonic and adult wound healing. First, the inventors examined whether Agrin orchestrated actomyosin dynamics at the leading edge during wound stress. Wounding generated robust actomyosin cables within 4 h in control keratinocytes (FIG. 9a). Agrin deprived cells lacked these actomyosin cables which were restored by exogenously treated Agrin (FIG. 9a). This actomyosin cable network is underlined by the dramatic induction of phosphorylated myosin light chain (pMLC) within 2 hours post-wound injury in control cells that bestows enhanced contractility and migration velocity (FIG. 9a-b). Agrin depleted cells consistently showed a severe dampening of pMLC activation within 2 h post-wounding, and this was restored by sAgrin pre-treatment (FIG. 9b). Recombinant sAgrin treatment led to strong recruitment of pMLC to wound edges forming stable actomyosin cables that were abolished by the MLC inhibitor, blebbistatin (FIG. 9c). Second, the formation of robust actomyosin cables was integral to collective cell migration as blebbistatin treatment abolished the sAgrin induced migration of HaCaTs post-wound injury (FIG. 9d). These lines of evidence support that Agrin organizes actomyosin architecture at the leading front for sustaining migratory potential of cells post-wounding.

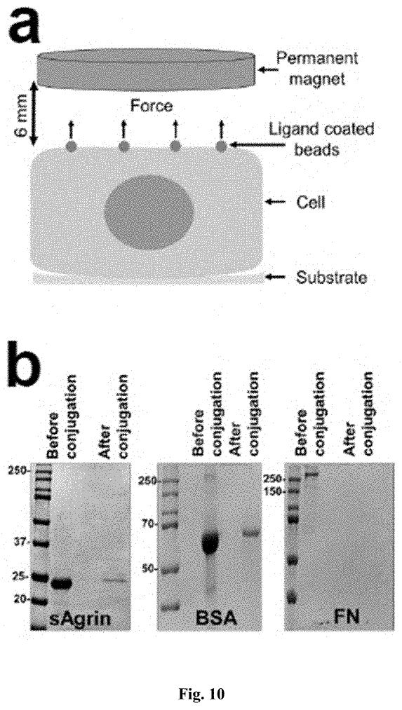

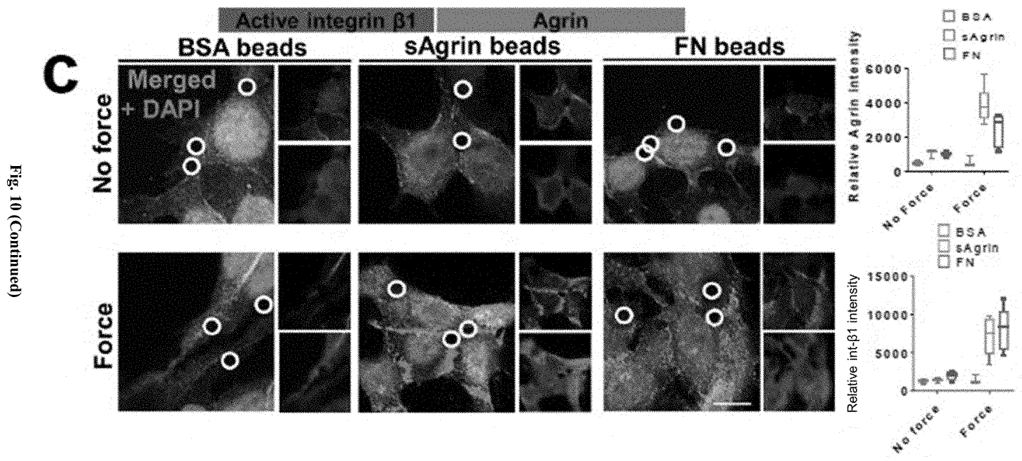

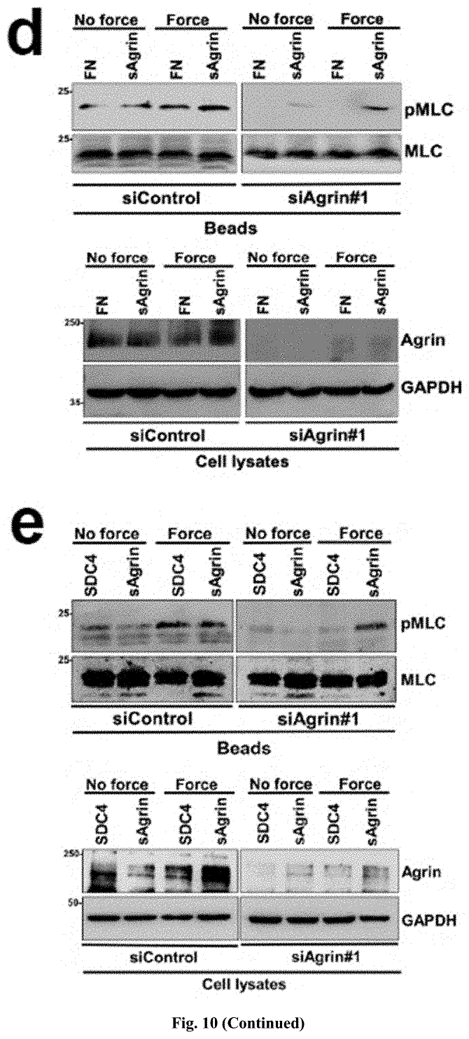

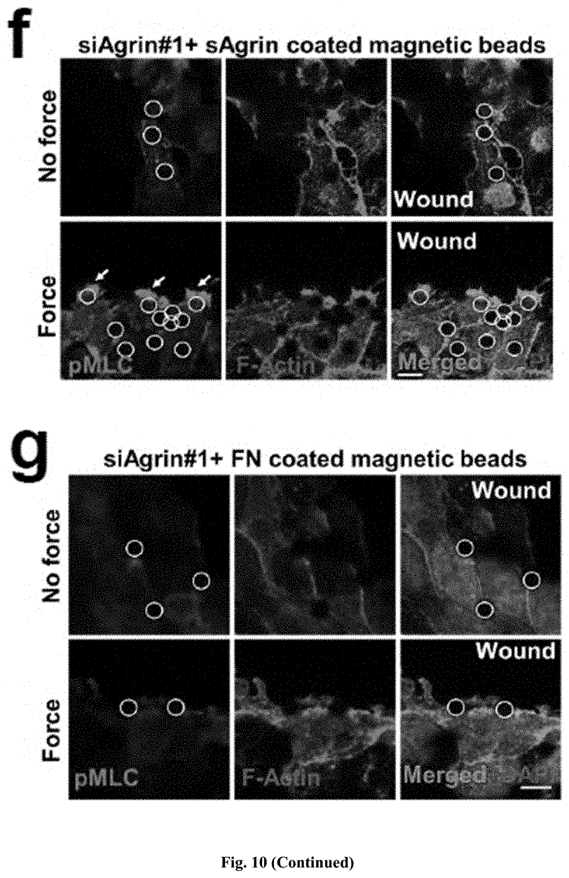

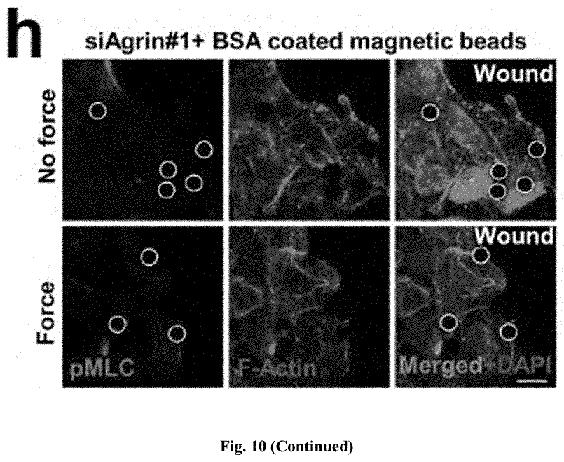

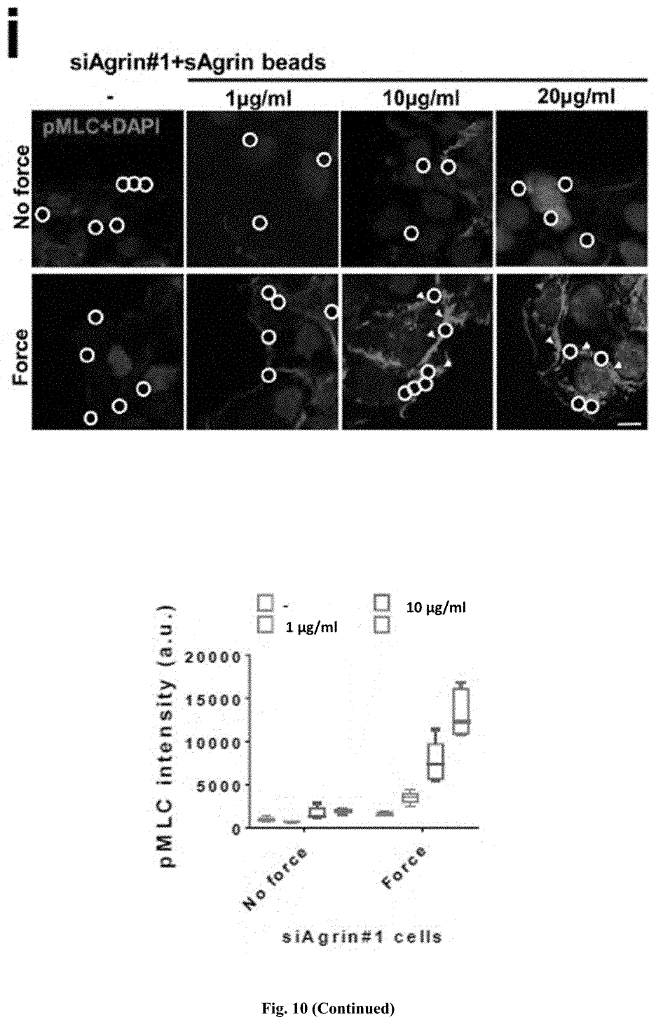

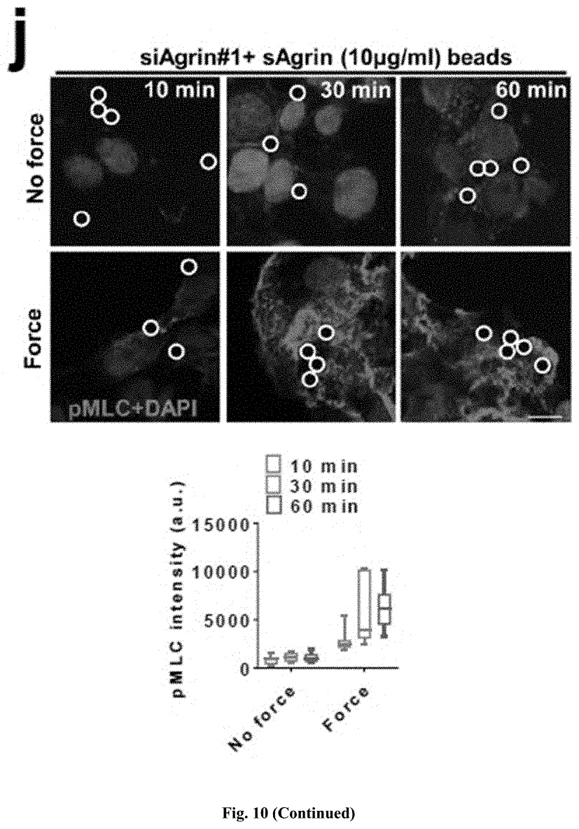

To gain deeper insights on how cellular mechanoperception is calibrated through actomyosin engagement by an Agrin-induced force recognition mechanism, the inventors used a permanent magnet to apply mechanical force on wounded keratinocytes via ligand coated magnetic beads that simulated the effects of abnormal mechanical force experienced by the wound microenvironment. In this setup, magnetic beads were conjugated with control proteins (Bovine Serum Albumin-BSA or Fibronectin-FN) and sAgrin, respectively, and were subsequently allowed to bind to the cell surface. A permanent magnet placed 6 mm above the cells in culture plate ensured a sustained force of 200 pN was applied for 30 min post-wound scratch through the ligand coated beads (FIG. 10a). The efficacious ligand conjugation to the beads via covalent bonding is shown by the abrupt reduction of remnant free proteins post-conjugation (FIG. 10b). Similar to the integrin ligand FN, sAgrin beads substantially increased activated integrin β1-Agrin localization in control migrating keratinocytes, suggesting an enhanced mechanotension is induced upon localized force application (FIG. 10c). Actomyosin cables were detected at 30 min post-wounding in control cells without any additional magnetic force (FIG. 9e, no force panel); however, application of exogenous force further surged pMLC recruitment localized near sAgrin beads forming robust actomyosin cables (FIG. 9e, force panel). In stark contrast, Agrin knockdown cells exhibited poor localization of pMLC and actomyosin cables under normal conditions post-wounding, as reported in FIG. 9a, respectively (FIG. 9e). It was anticipated that transient force transmission by sAgrin, in part, should render greater actomyosin recruitment as a manifestation of enhanced mechanoresponse to the wounded cells. Accordingly, application of force with sAgrin for 30 min led to a ˜2 folds increase of pMLC around the bead vicinity that partially rescued the actomyosin cables activity in Agrin depleted cells without any prior exposure to sAgrin in the culture media (FIG. 9e-f). Upon force application to wounded cells, sAgrin induced greater pMLC recruitment in control cells compared to FN, but similar to that induced by Syndecan-4 (SDC4) (FIG. 10d-e, siControl panels). Although FN stimulated integrins as global mechanosensors and syndecan-4 tuned cell mechanics, these were rather insufficient to rescue myosin mechanotension in Agrin deprived cells. Essentially, the reduced pMLC in Agrin depleted cells was only restored by force transduced by sAgrin (and not by FN and SDC4) beads (FIG. 10d-e, siAgrin panel). Likewise, compared to FN or BSA beads, sAgrin coated beads enhanced pMLC recruitment upon force application in Agrin depleted cells at 30 min post-wounding (FIG. 10f-h). The specificity of Agrin as a mechanotransducer was additionally tested by the following strategies: first, increasing sAgrin coated beads exerted greater pMLC recruitment at 30 min post-wound scratch in a dose-dependent fashion (FIG. 10i). Second, temporal increase of force application via constant stimulation by sAgrin coated beads up to 60 min led to an enhanced pMLC mechanoperception following wound-injury (FIG. 10j). These results advocate that Agrin acts as a mechanotransducer of extrinsic force sufficient to overhaul cytoskeletal dynamics required for collective migration following wound injury.

In addition to the loss of ECM components, keratinocytes often navigate through wounded areas adapting to large-scale changes to their morphology and cytoskeleton under different geometrical tensions. To simulate whether Agrin influences keratinocytes' ability to shift their cytoskeletal tension upwards upon exposure to geometrical constraints, normal HEK cells were cultured in crossbow shaped FN patterns of different surface areas. The FN coated crossbow micropatterns force the cells to assume a polarized orientation with F-actin stress fibers originating from the dorsal arc and extensive actomyosin stress fiber bundling at the transverse arc towards the base. Smaller (800μ²) fibronectin patterns compressed cells even further leading into severe loss of F-actin stress fibers (FIG. 9g). Despite within small confinements, sAgrin supplemented crossbow patterns partially rescued F-actin stress fibers and higher pMLC recruitment to the transverse arc (FIG. 9g). This effect of sAgrin induced stress fiber formation in geometrically constrained HEK cells was further accentuated in large crossbow (1600μ²) patterns, where cells demonstrated a prominent tension signature comprising of elongated F-actin stress fibers and actomyosin tension at the transverse arc, respectively (FIG. 9g, lower panels). Of note, sAgrin conferred significantly higher tension curvatures that were enriched with activated MLC at the transverse arcs of cells in large crossbow patterns (FIG. 9h). Importantly, F-actin stress fibers and pMLC enriched transverse arc tension bundles were completely abrogated in Agrin depleted cells (FIG. 9i, first and second panels, respectively). Interestingly, sAgrin incorporated within the FN crossbow matrix restored the tension signature of F-actin stress fiber network and pMLC enriched transverse arcs (FIG. 9i, third panel). Together, these results suggest that tension sensing mechanisms are conferred by Agrin in keratinocytes under geometric constraints, thereby adapting them towards an ECM matrix that is favorable for the healing program.

MMP12 as a Mediator of Agrin-Mechanotransduction Following Wound Injury

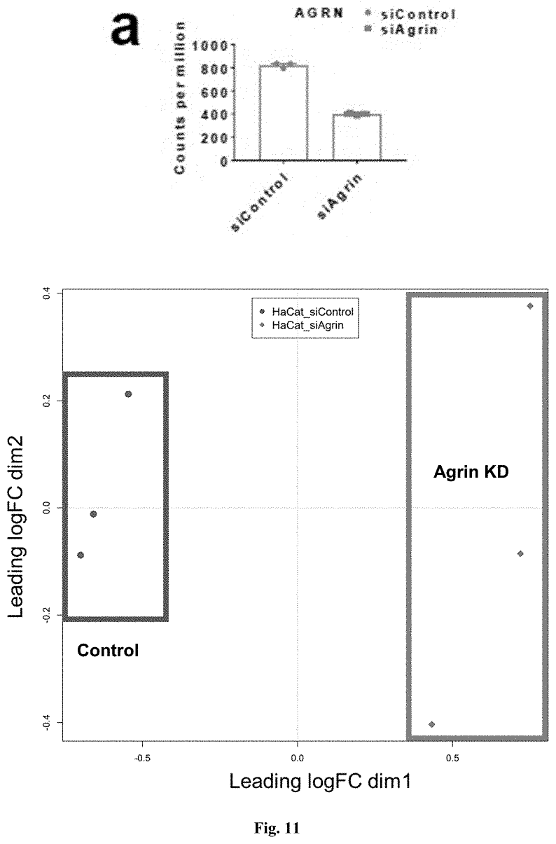

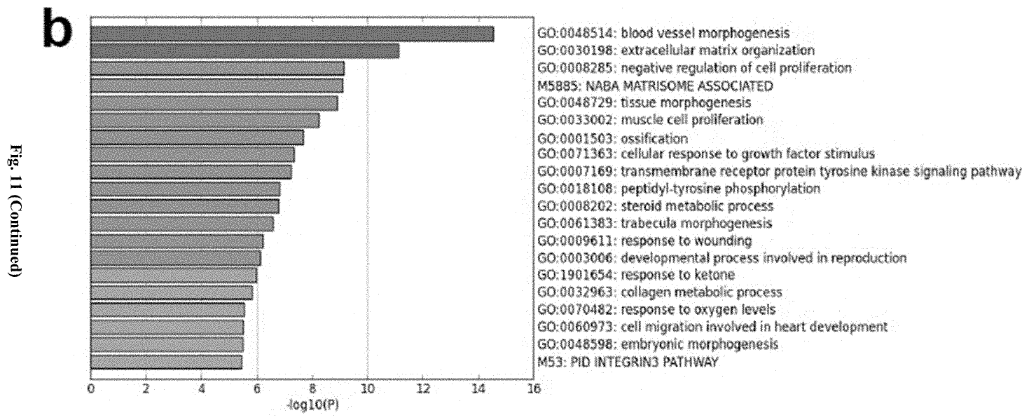

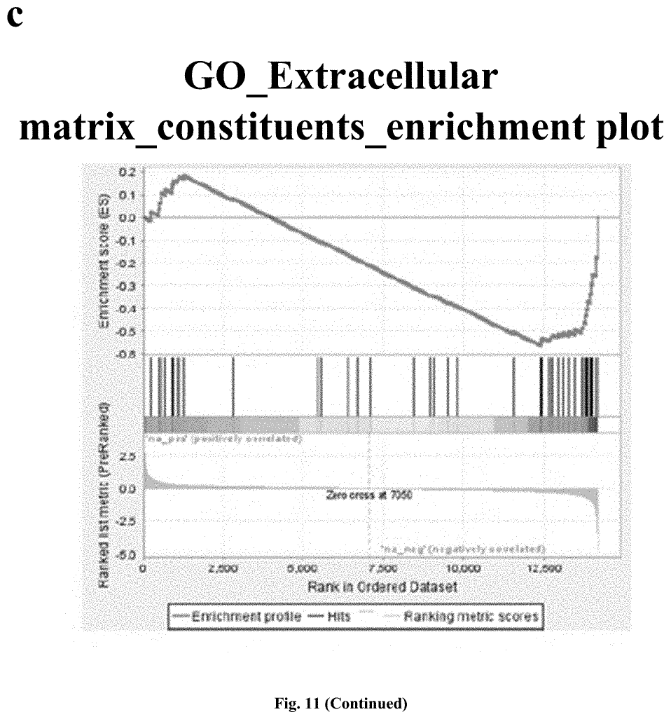

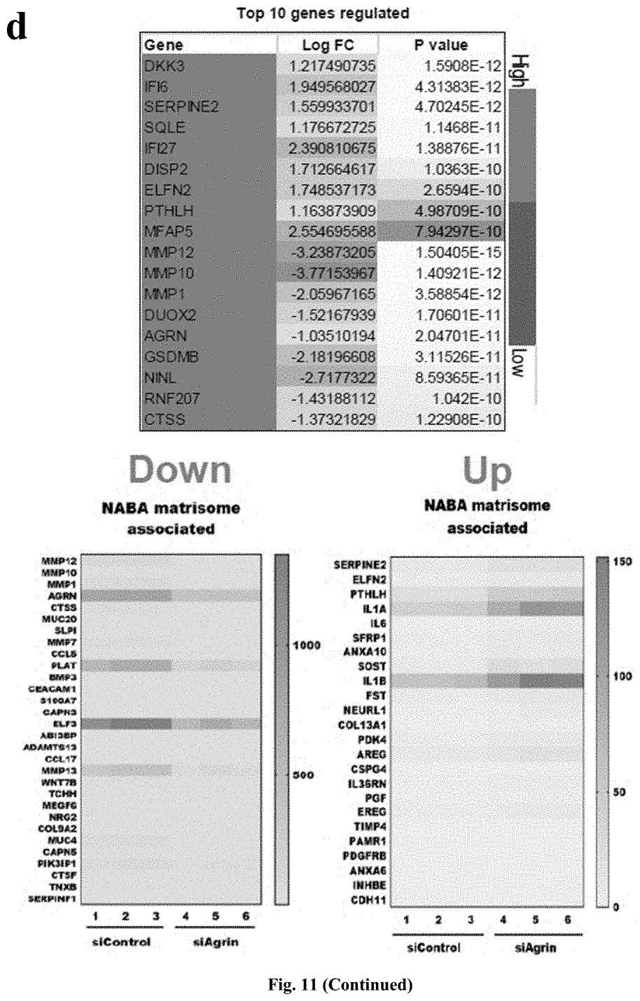

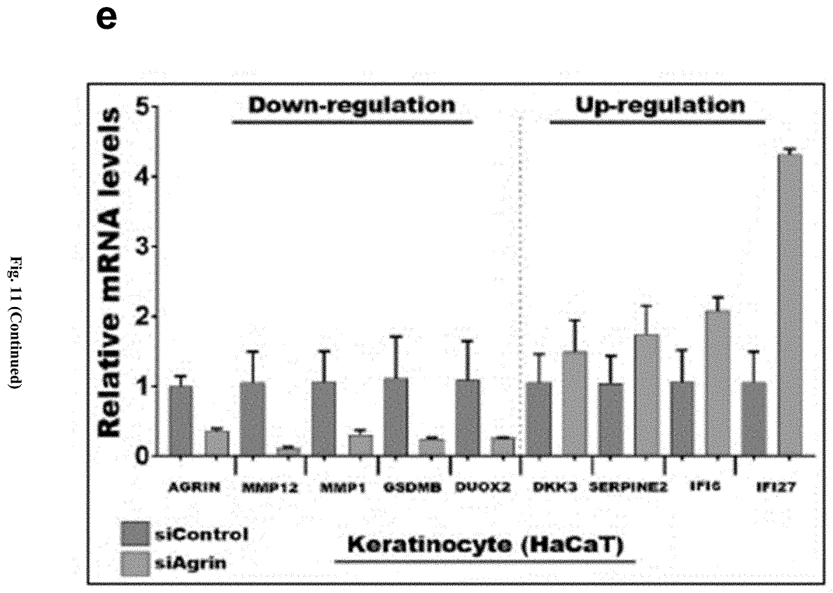

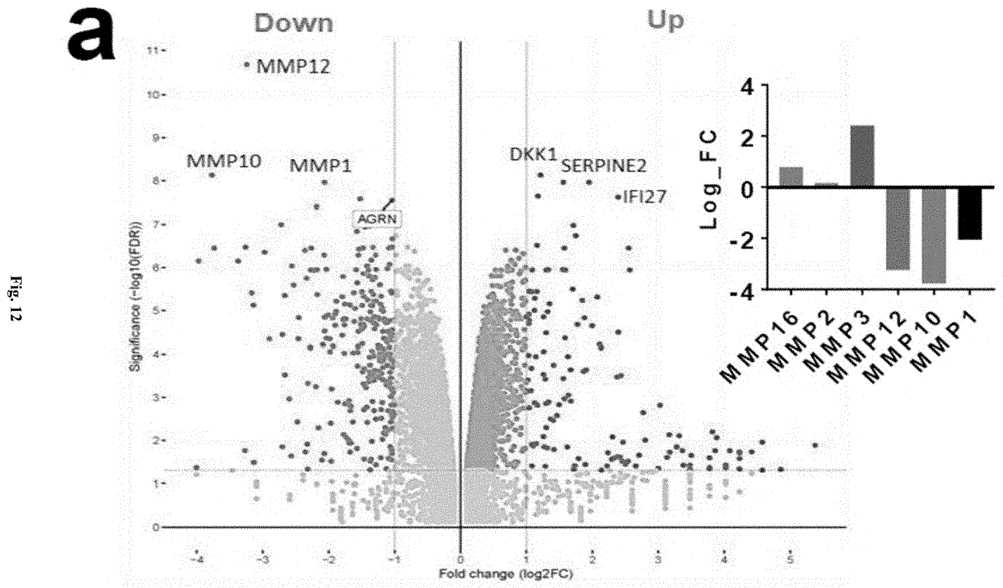

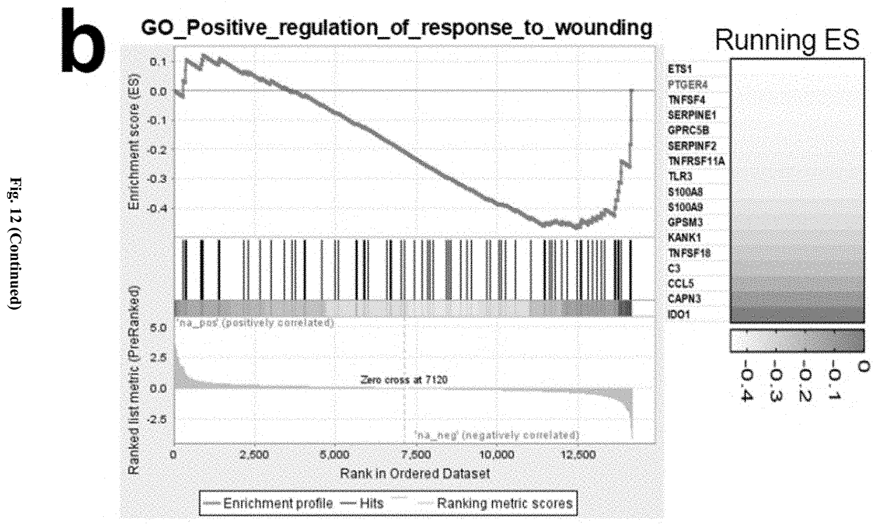

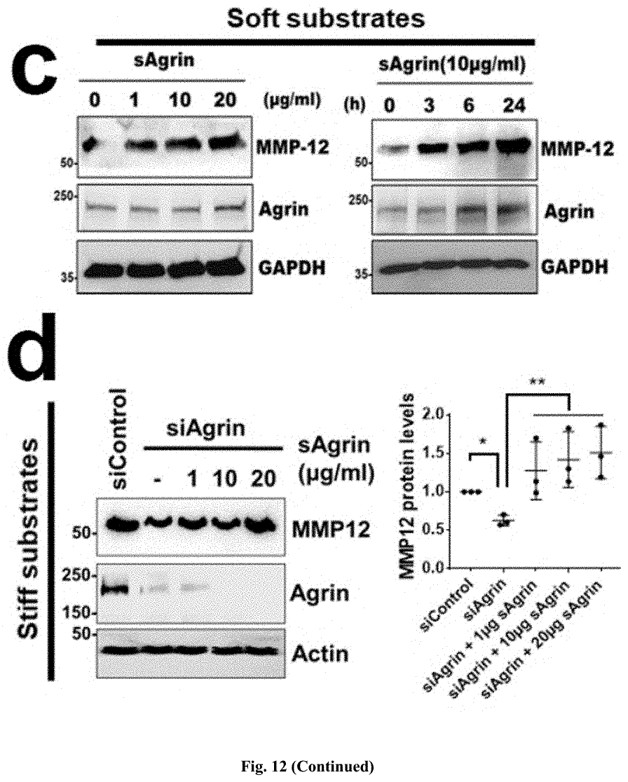

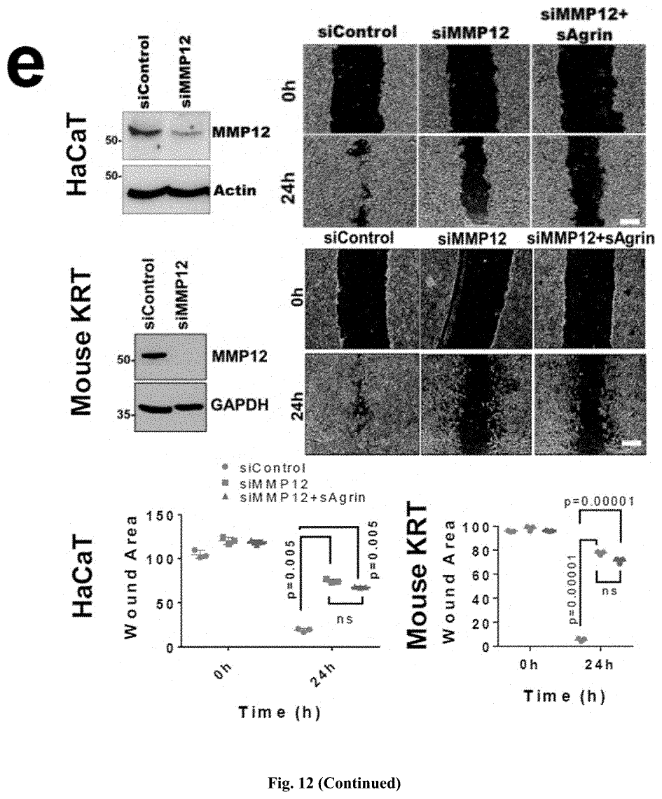

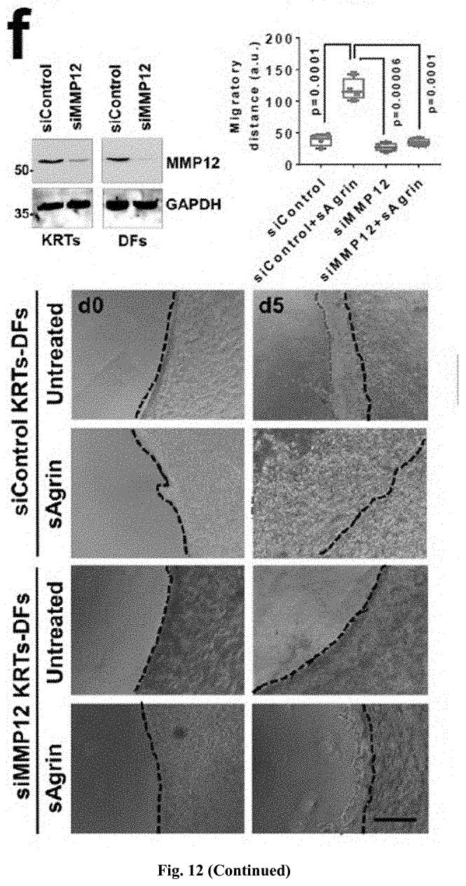

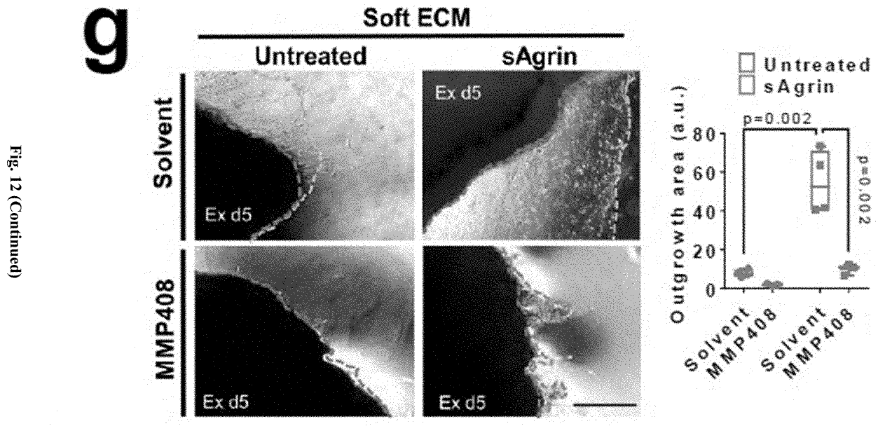

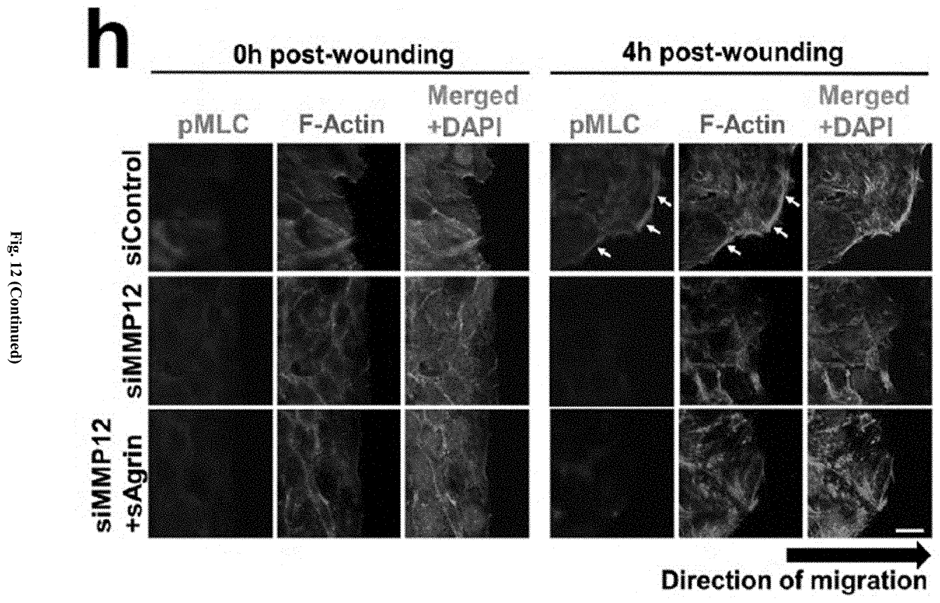

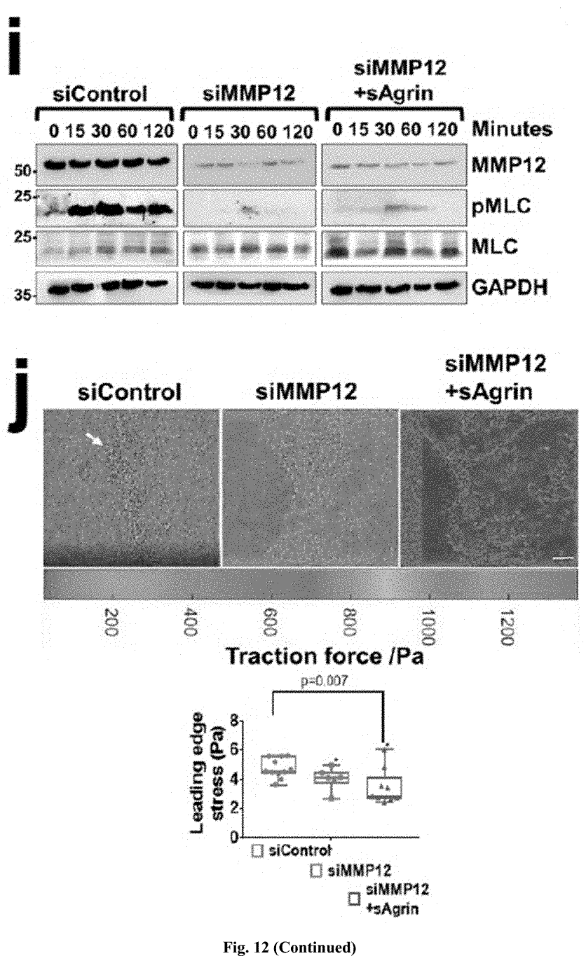

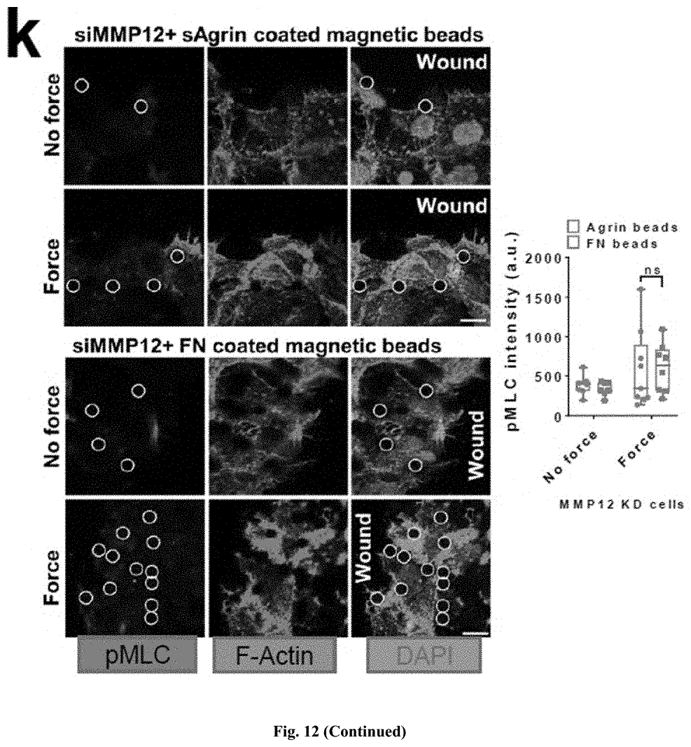

To identify the downstream effectors of Agrin mediating mechanotension in keratinocytes upon wound injury, the inventors performed transcriptome-wide comparison via RNAseq analysis in control versus Agrin depleted cells cultured in plastics that mimic a highly stiff ECM (FIG. 11a). Multidimensional scaling (MDS) revealed distinct gene profiles between the control and Agrin depleted bulk RNA population (FIG. 11a). Upon stratifying a staggering number of genes that were differentially regulated by Agrin in stiff ECM, a few highly significant clusters using gene ontology (GO) analysis were selected (FIGS. 12a, 11b, Tables 2 and 3). GO, network and Gene set enrichment analysis (GSEA) revealed that the majority of genes that were drastically down-regulated upon Agrin knockdown belonged to ECM modulation including matrisome-associated and structural components of ECM (FIGS. 11b-d). In the top 10 significant genes that were regulated by Agrin, several matrisomal proteins including the matrix metalloproteases (MMPs) were consistently reduced while others such as Serpine2, Interleukin 1a-b, were upregulated upon Agrin depletion (FIGS. 12a and 11d). In line with effects on cell migration, GSEA analysis also revealed a global reduction in gene cluster that positively regulated wound healing (FIG. 12b). We validated the mRNA expression(s) of selected set of up- and down-regulated genes (FIG. 11e). Expression of mRNAs of several MMPs including MMP12, 10 and 1 were found to be significantly reduced upon Agrin knockdown.

TABLE 2
Pro-inflammatory cytokines and chemokines
primers used in the study

| Target Gene | Forward/ | SEQ | |
|---|---|---|---|
| (Mouse) | Reverse | ID NO | Sequence (5′ to 3′) |
| IL-18 | Forward | 68 | GCCTCAAACCTTCCAAATCA |
| | Reverse | 69 | TGGATCCATTTCCTCAAAGG |
| TGF-BETA 1 | Forward | 70 | CCTGTCCAAACTAAGGC |
| | Reverse | 71 | GGTTTTCTCATAGATGGCG |
| VEGF | Forward | 72 | GTACCTCCACCATGCCAAGT |
| | Reverse | 73 | TCACATCTGCAAGTACGTTCG |
| IL1beta | Forward | 74 | CCTTCCAGGATGAGGACATGA |
| | Reverse | 75 | TGAGTCACAGAGGATGGGCTC |
| TLR3 | Forward | 76 | CCTCCAACTGTCTACCAGTTCC |
| | Reverse | 77 | GCCTGGCTAAGTTATTGTGC |
| MIP-1α | Forward | 78 | TGAATGCCTGAGAGTCTTGG |
| | Reverse | 79 | TTGGCAGCAAACAGCTTATC |
| MIP-1β | Forward | 80 | TGCTCGTGGCTGCCTTCT |
| | Reverse | 81 | CAGGAAGTGGGAGGGTCAGA |
| MIP-2 | Forward | 82 | CGCCCAGACAGAAGTCATAG |
| | Reverse | 83 | TCCTCCTTTCCAGGTCAGTTA |
| KC | Forward | 84 | TGCACCCAAACCGAAGTCAT |
| | Reverse | 85 | TTGTCAGAAGCCAGCGTTCAC |
| TARC | Forward | 86 | CAAGCTCATCTGTGCAGACC |
| | Reverse | 87 | CGCCTGTAGTGCATAAGAGTCC |
| RANTES | Forward | 88 | CACCACTCCCTGCTGCTT |
| | Reverse | 89 | ACACTTGGCGGTTCCTTC |
| MCP-1 | Forward | 90 | TAGGCTGGAGATCTACAAGAGG |
| | Reverse | 91 | AGTGCTTGAGGTGGTTGTGG |
| MIP-3ß | Forward | 92 | AGCCTTCCGCTACCTTCTTA |
| | Reverse | 93 | GCTGTTGCCTTTGTTCTTGG |
| I-309 | Forward | 94 | CGTGTGGATACAGGATGTTGACAG |
| | Reverse | 95 | AGGAGGAGCCCATCTTTCTGTAAC |
| TRIF | Forward | 96 | GGTTCACGATCCTGCTCCTGAC |
| | Reverse | 97 | GCTGGGCCTGAGAACACTCAAG |
| GAPDH | Forward | 98 | TGAGCAAGAGAGGCCCTATC |
| | Reverse | 99 | AGGCCCCTCCTGTTATTATG |
| TNF-α | Forward | 100 | GCCTCTTCTCATTCCTGCTTG |
| | Reverse | 101 | CTGATGAGAGGGAGGCCATT |
| IL-6 | Forward | 102 | ACGGCCTTCCCTACTTCACA |
| | Reverse | 103 | CATTTCCACGATTTCCCAGA |
| ß-actin | Forward | 104 | CGTGCGTGACATCAAAGAGAA |
| | Reverse | 105 | TGGATGCCACAGGATTCCAT |

TABLE 3
Small interfering RNA (siRNA) sequences

| Name | Company | Primer Name | SEQ ID NO | Sequence (5′ to 3′) |
|---|---|---|---|---|
| Human | Invitrogen | AGRNHSS139721 | 106 | CCUUUGUCGAGUACCUCAACGCUGU |
| AGRN | (Stealth) | | 107 | ACAGCGUUGAGGUACUCGACAAAGG |
| | | AGRNHSS180123 | 108 | CAUACGGCAACGAGUGUCAGCUGAA |
| | | | 109 | UUCAGCUGACACUCGUUGCCGUAUG |
| | | AGRNHSS180124 | 110 | GCGAUUUAUGGACUUUGACUGGUUU |
| | | | 111 | AAACCAGUCAAAGUCCAUAAAUCGC |
| Mouse | Invitrogen | AgrnMSS201833 | 112 | GGAGACCUGCCAGUUUAACUCUGUA |
| AGRN | (Stealth) | | 113 | UACAGAGUUAAACUGGCAGGUCUCC |
| | | AgrnMSS201834 | 114 | AGGUUCCCUUCAGGUGGGCAAUGAA |
| | | | 115 | UUCAUUGCCCACCUGAAGGGAACCU |
| | | AgrnMSS201835 | 116 | CCAAAGUCCUGUGAUUCCCAGCCUU |
| | | | 117 | AAGGCUGGGAAUCACAGGACUUUGG |
| Mouse | Invitrogen | MMP12MSS206678 | 118 | GGAGCUCACGGAGACUUCAACUAUU |
| MMP12 | (Stealth) | | 119 | AAUAGUUGAAGUCUCCGUGAGCUCC |

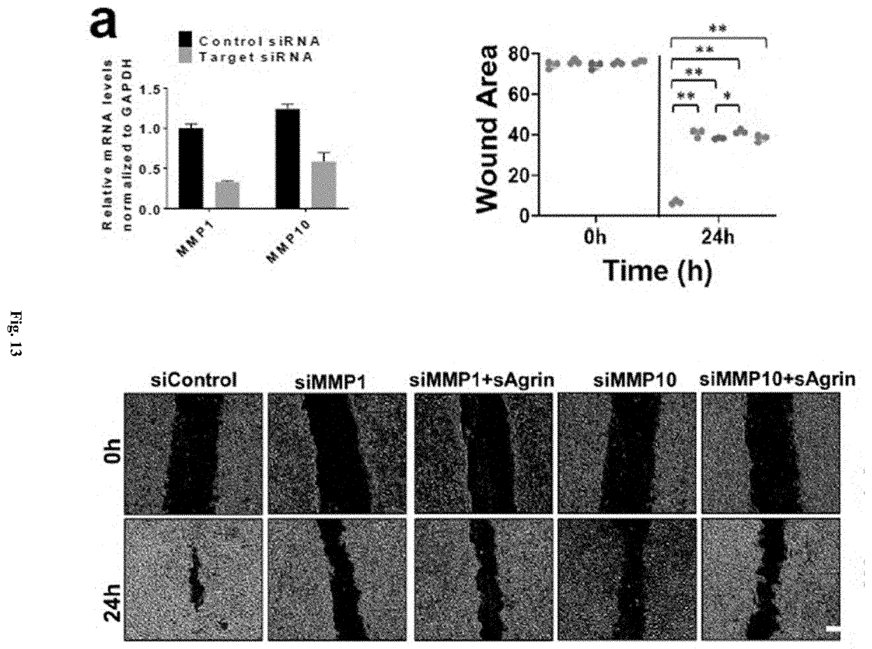

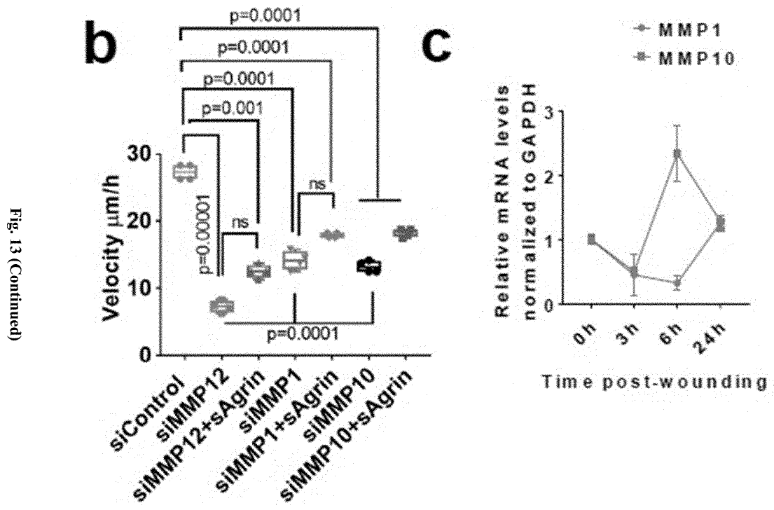

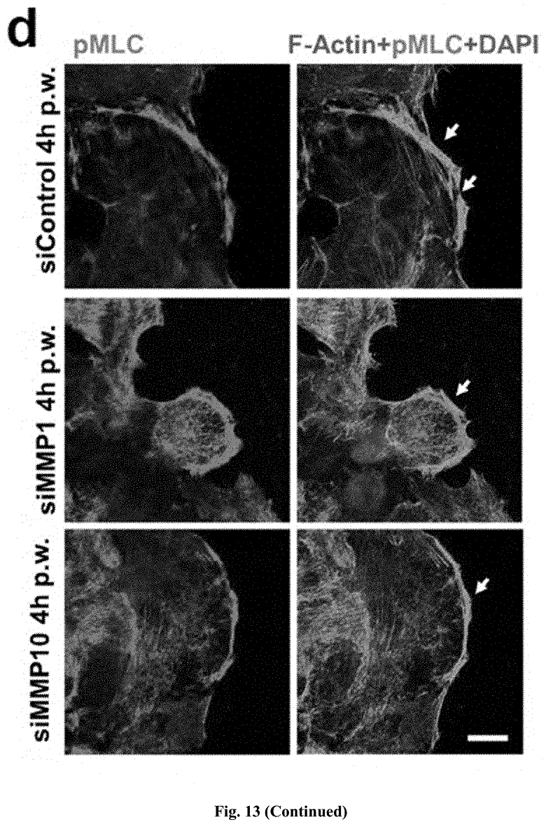

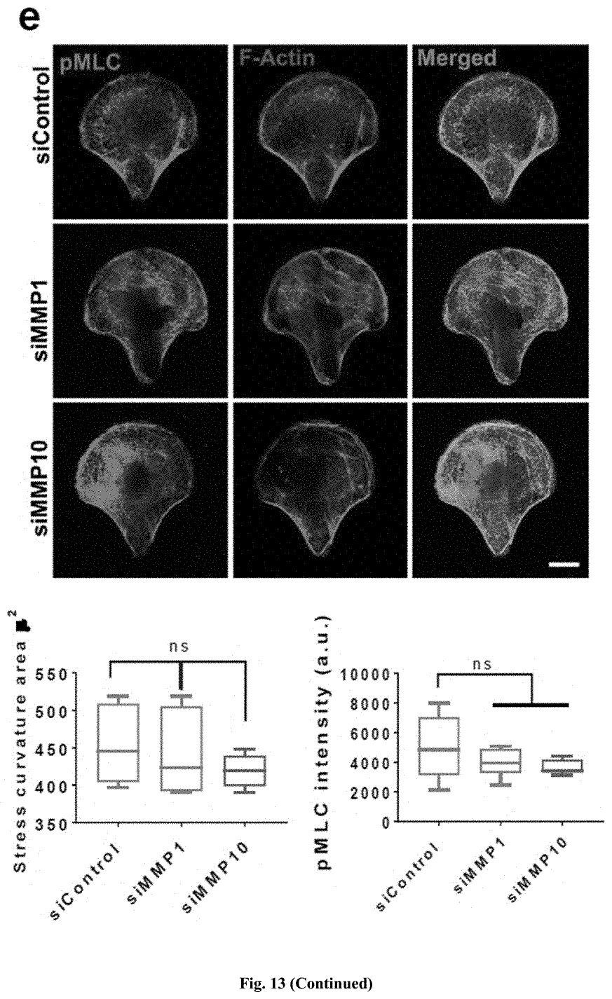

The next set of experiments focused on identifying the key MMPs that mediate Agrin's mechanical functions in the wound environment. First, it was evaluated whether MMP 1, 10 and 12 independently affected keratinocyte migration following wound-scratch. Accordingly, knocking down MMP 1 and 10 reduced keratinocytes' migration that was not restored by sAgrin treatment (FIG. 13a). In comparison, MMP12 knockdown showed the maximal inhibition of migration velocities post-injury (FIG. 13b). Strikingly, it was not observed that the mRNA levels of MMP1 and MMP10 were consistently upregulated following wound injury in HaCaT cells (FIG. 13c). Most importantly, MMP1 or MMP10 depletion had no effects on reducing the actomyosin tension at the wound edges (FIG. 13d). When cultured on large crossbow patterns, MMP1 or MMP10 depleted cells did not show any reduction of tension signatures of F-actin stress fiber network and pMLC enriched transverse arcs (FIG. 13e). Despite being regulated by Agrin, these observations rule out any significant role(s) of MMP1 or 10 as downstream mediators restoring the wound healing mechanics.

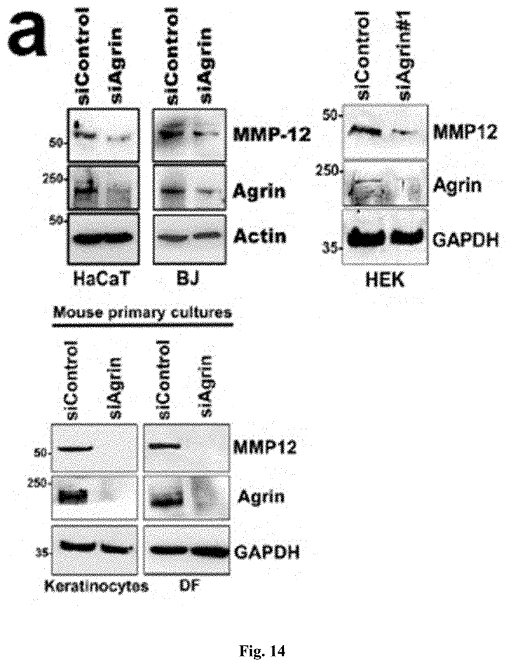

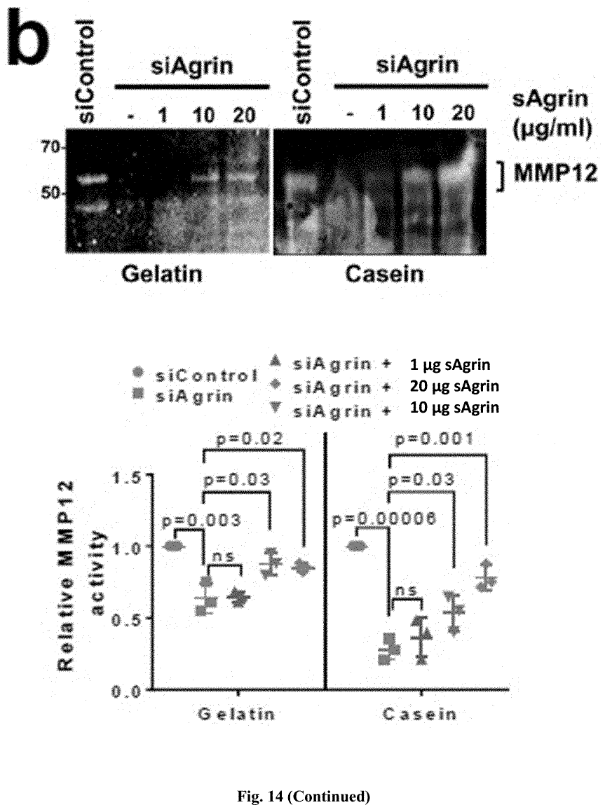

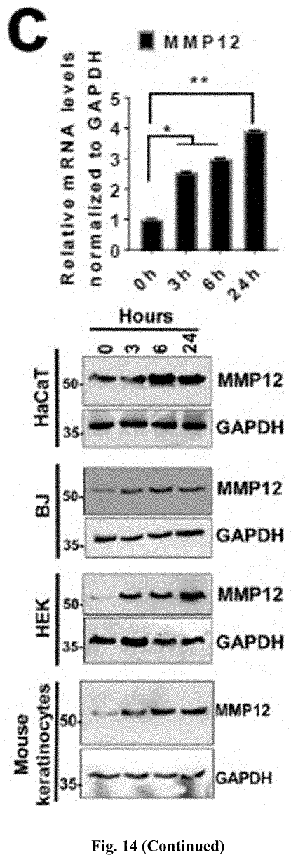

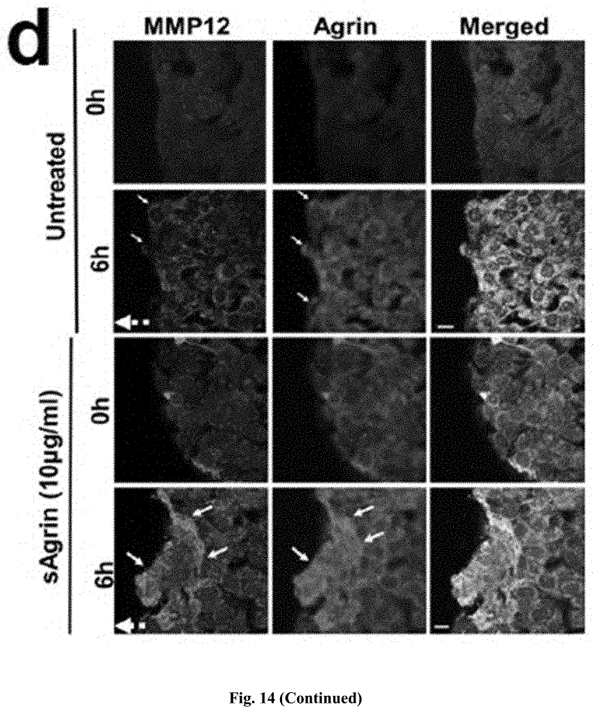

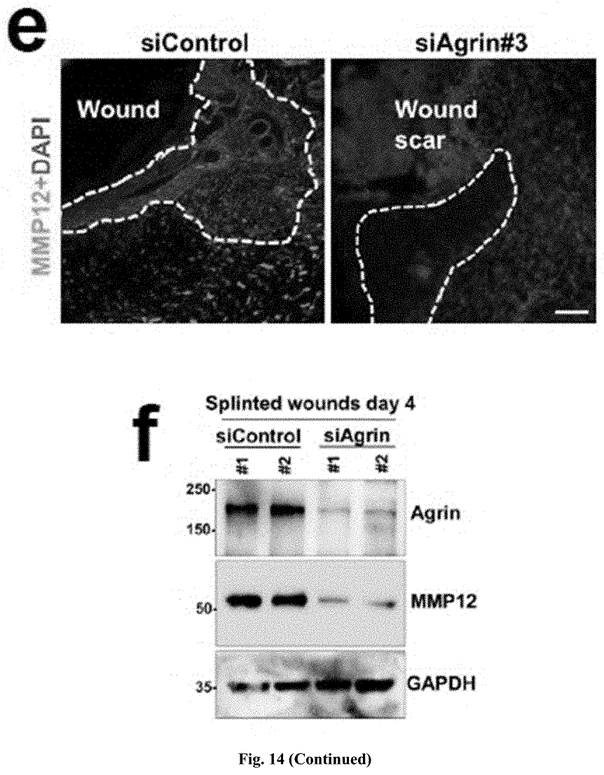

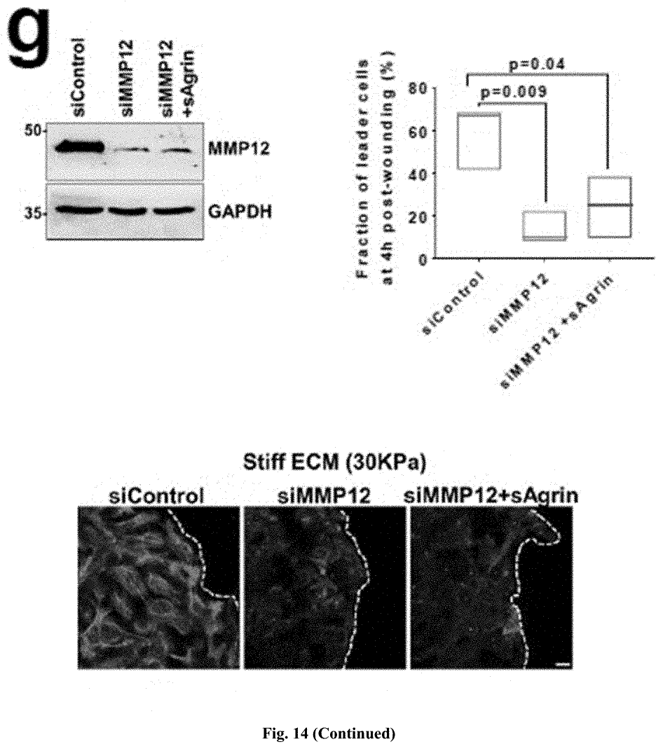

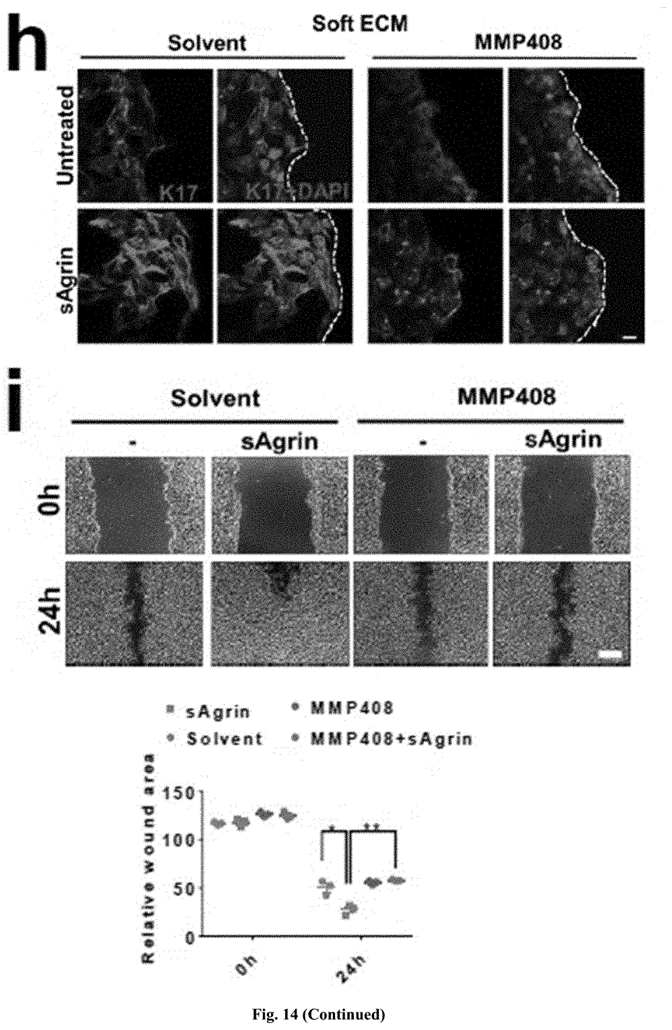

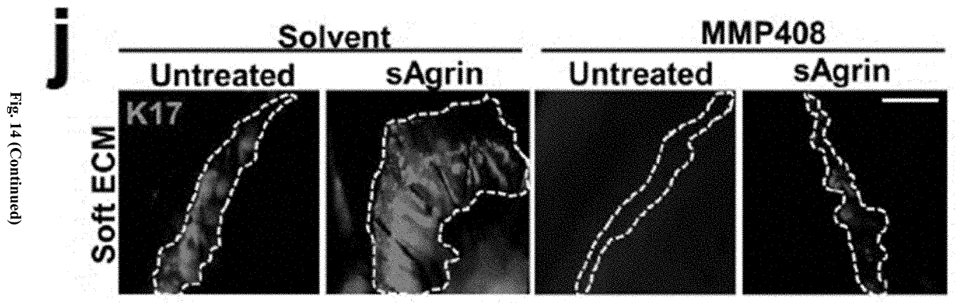

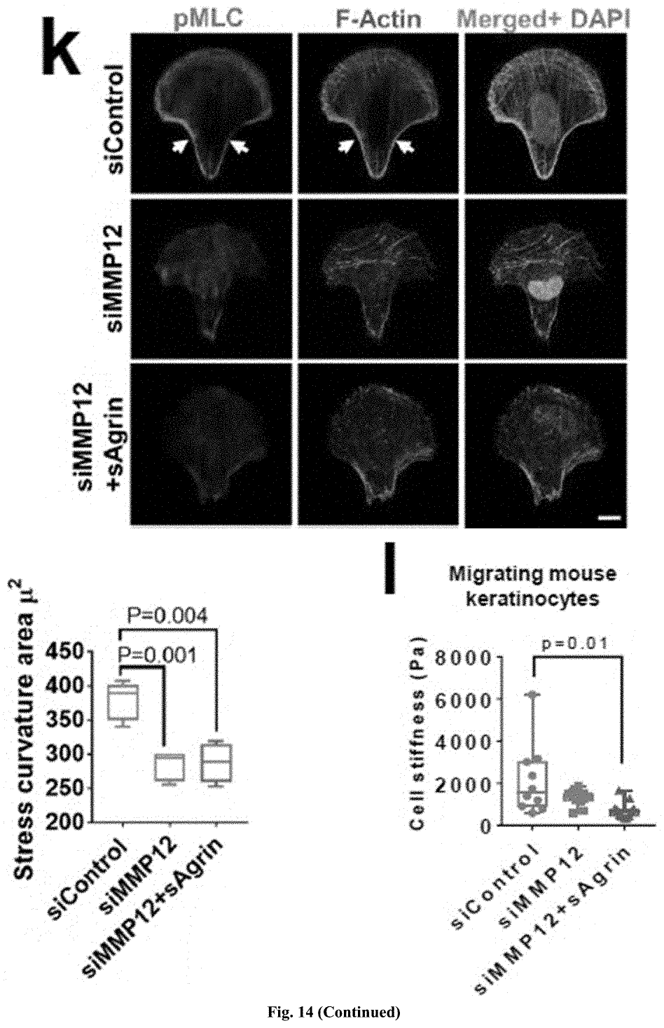

MMP12 emerged as the most significant potential candidate for mediating Agrin's mechanotension in wound repair. Similar to its mRNA levels, MMP12 protein levels were significantly reduced upon Agrin depletion in a panel of immortalized and primary keratinocytes and dermal fibroblasts cultures (FIG. 14a). Due to a decrease in both mRNA and protein levels upon Agrin depletion, a loss of MMP12's gelatin and casein degradation catalytic activity was obvious in Agrin silenced keratinocytes which was restored by sAgrin supplementation in a dose-dependent pattern (FIG. 14b).

It was next determined whether the activation of MMP12 is a consequence of Agrin's sensitization towards ECM rigidity in a wound injury associated Agrin-rich environment. First, HaCaT cells were cultured in soft substrates (0.8 kPa) alone or those with increasing concentration of supplemented sAgrin. Importantly, soft substrates incorporated with sAgrin strongly stabilized MMP12 protein levels in a dose-dependent fashion (FIG. 12c, first panel). Enhancing the exposure time of keratinocytes cultured in soft matrix conditioned with sAgrin also increased MMP12 levels, suggesting that Agrin closely mimics ECM rigidity cues to positively regulate MMP12 levels in keratinocytes (FIG. 12c, right panel). On the contrary, the depletion of Agrin in cells experiencing stiff ECM led to a significant reduction in MMP12 levels (FIG. 12d). Agrin knockdown cells restored their MMP12 levels upon sensing sAgrin cues from the stiff ECM in a dose-dependent manner (FIG. 12d). Second, unlike MMP1 or 10, wound injury strongly activated mRNA (˜4 folds) and protein levels of MMP12 that closely mirrored that of Agrin overexpression in several skin cells (FIGS. 14c, 13c and FIG. 1b). As early as 6 h post-wound scratch, MMP12 and Agrin were detected within cells at the leading migrating front; this effect was further heightened by sAgrin supplementation, hence, simulating a favorable platform for collective keratinocyte migration following wound injury (FIG. 14d). The migrating epithelial tongue mediating in vivo wound closure effectively expressed MMP12 in control siRNA treated mouse skin (FIG. 14e). Localized Agrin depletion significantly deprived MMP12 levels within the attenuated epithelial tongues that subsequently failed to close the wound (FIG. 14e). In the splinted wound healing in vivo models, Agrin depletion also strongly suppressed MMP12 expression within day 4 post-injury that accounted for poor healing rates (FIG. 14f). This was recapitulated in vitro where MMP12 knockdown mimicked the loss in migration of human immortalized and primary mouse keratinocytes post-wound scratch similar to that exhibited by Agrin depleted cells (FIG. 12e). Cumulatively, these shreds of evidence indicate that a wound-stressed Agrin microenvironment activates MMP12 for collective keratinocyte migration.

Several snippets of observations further imply that MMP12 mediated the Agrin induced collective migration post-wounding. Firstly, sAgrin failed to stimulate migration in MMP12 depleted human and mouse keratinocytes (FIG. 12e). Secondly, knockdown of MMP12 in keratinocytes experiencing stiff substrates (30 kPa) severely hampered K17 expressing migrating cells and this was not restored by the nourishment of sAgrin (FIG. 14g). Thirdly, short term treatment of keratinocytes with MMP408, a specific MMP12 inhibitor, blunted the migration of sAgrin induced K17 expressing keratinocytes in soft substrates and under normal cell culture conditions (FIGS. 14h-i). Fourthly, sAgrin incorporated soft matrix stimulated mouse keratinocyte migration over dermal fibroblasts post-injury as reported previously in 3D stiffness-dependent migration assays (FIG. 12f, first and second panels). In contrast, depleting MMP12 in mouse keratinocytes and underlying dermal fibroblasts completely abrogated the keratinocyte migration which was not even rescued by sAgrin supplemented compliant matrices (FIG. 12f, third and fourth panels). Ex vivo, the enhanced K17 expressing migratory outgrowth from the mouse skin explants induced by sAgrin supplemented in soft matrix was abruptly abolished by the presence of the MMP408 inhibitor (FIGS. 12g and 14j).

In reminiscence to the fact that MMP12 regulates Agrin-driven migration, it was rationalized MMP12 as a mediator of Agrin's mechanotension sensing in wound stressed keratinocytes. Accordingly, the next set of experiments was focused on determining the role of MMP12 in overhauling cytoskeletal tension in response to an Agrin-rich environment, as poised during early wound healing stages. The robust actomyosin cables generated in Agrin complacent keratinocytes post-wounding were severed by the depletion of MMP12 that accompanied with pronounced inhibition of pMLC at the wound edges (FIG. 12h). Even sAgrin treatment did not restore actomyosin cable network unmasking a conspicuous defect in cytoskeletal orientation in these MMP12 suppressed cells (FIG. 12h). Likewise, sAgrin treatment was unable to rescue the severely diminished pMLC levels in MMP12 depleted keratinocytes within the observed 2 h post-wounding (FIG. 12i). Moreover, compared to the control cells, MMP12 knockdown cells and those with sAgrin nourishment did not exhibit F-actin stress fibers and pMLC enrichment in the transverse arcs when geometrically stretched in large crossbow patterns (FIG. 14k), suggesting that overhauling cytoskeletal tension by Agrin during collective cell migration post-wound injury requires functional MMP12.

Due to impaired collective migration upon MMP12 inhibition, it was examined whether reduced cytoskeletal tension is a consequence of progressive weakening in the mechanotransducing abilities of Agrin arising from MMP12 inhibition. To this end, the inventors first mapped the cell stiffness of MMP12 suppressed mouse keratinocytes during at 4 h post-injury. Strikingly, MMP12 knockdown significantly reduced the stiffness of migrating keratinocytes which were not restored by sAgrin treatment (FIG. 14l). In addition to reduced cell stiffness, the traction stresses of MMP12 knockdown keratinocytes migrating on TFM substrates were partially reduced at the migrating front (FIG. 12j). Interestingly, the treatment of sAgrin failed to induce sufficient traction force at the migrating leading edge of MMP12 depleted keratinocytes (FIG. 12j). Compared to control cells, MMP12 knockdown inhibited the homogenous velocity distribution at the leading front which further decreased the fluid-elastic-like migration in sAgrin supplemented cells (FIG. 14m). These results suggest that Agrin engaged MMP12 to provide a fluidic migratory behavior to keratinocytes. Furthermore, the pMLC activation via Agrin induced force transmission in wounded keratinocytes was also dampened upon MMP12 depletion. Both FN and sAgrin mediated force transmission were unable to rescue the reduced pMLC intensity in MMP12 depleted cells at 30 min post-wounding (FIG. 12k). Together, these results uncover that a macroscopic deficit of actomyosin tension in MMP12 depleted cells abrogated their mechanoperceiving ability conferred by Agrin following skin injury. Besides, the Agrin mechanotransduction in the wound environment contributing towards accelerated migration of keratinocytes and wound closure is largely dependent on MMP12.

Agrin Fails to Heal Wounds in MMP12 Deficient Mouse Skin

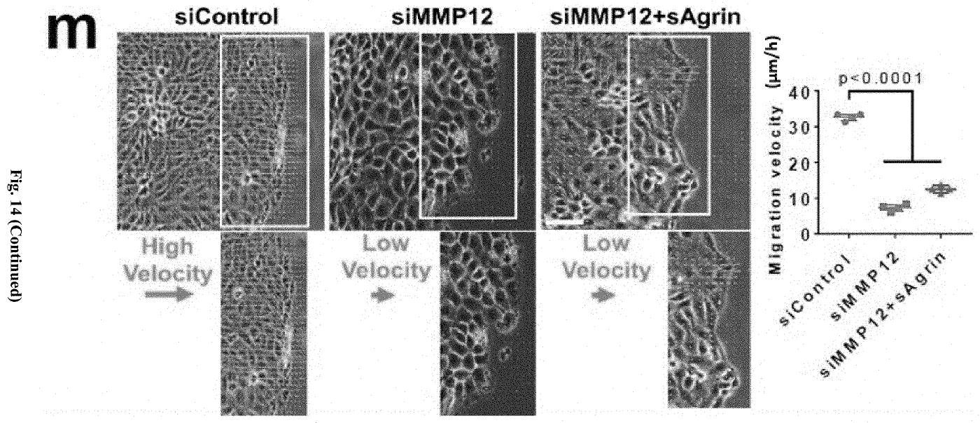

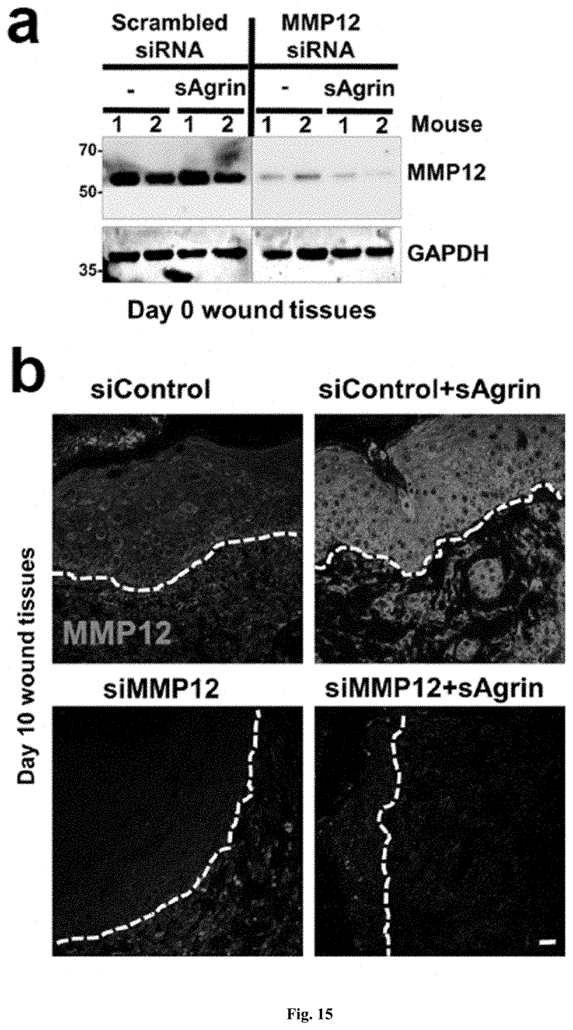

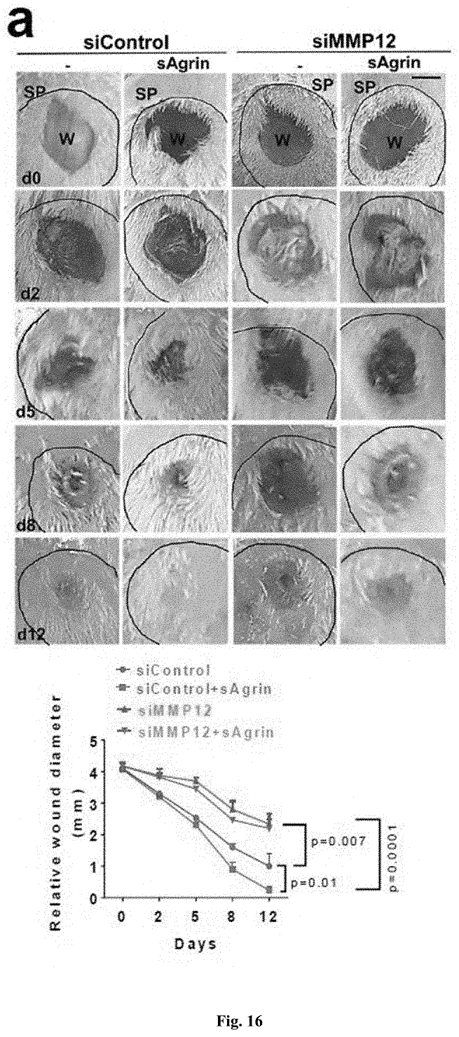

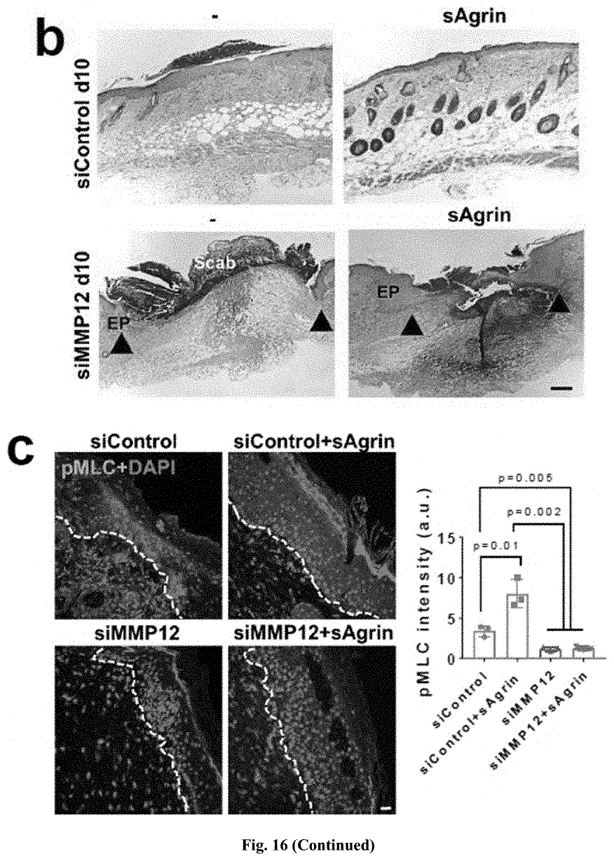

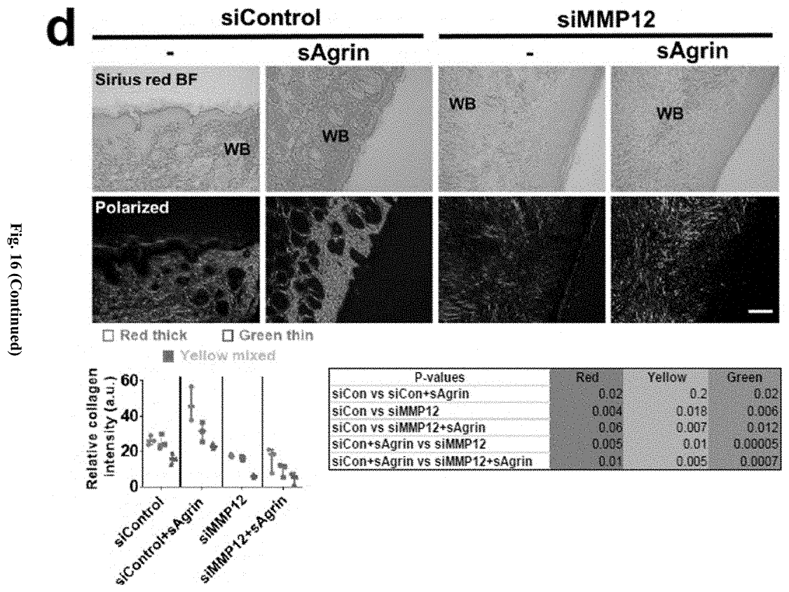

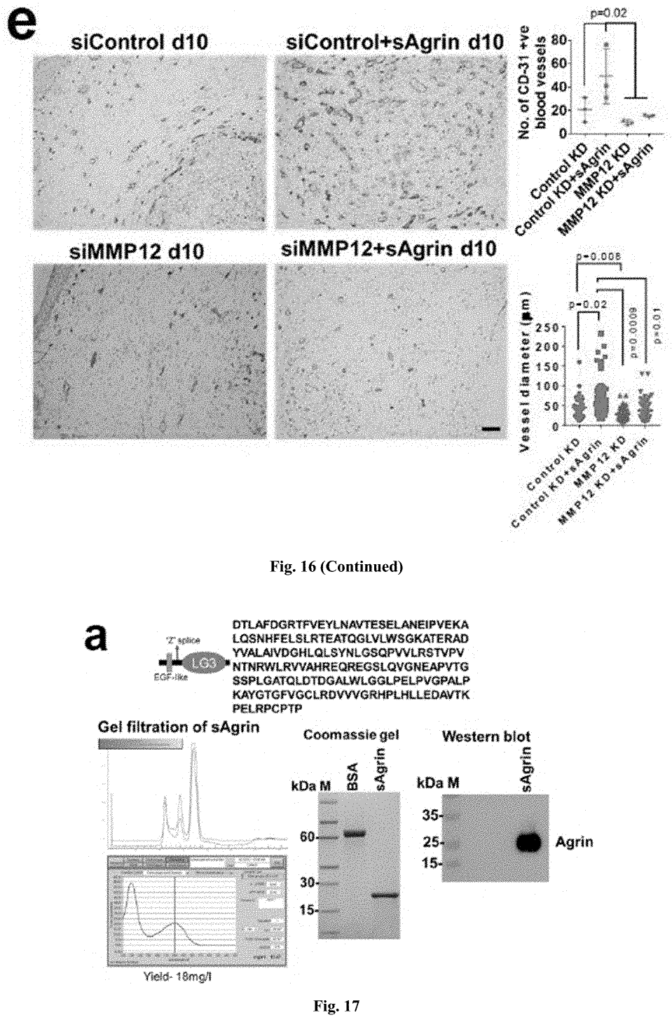

To test whether sAgrin bestows a mechanically competent wound healing environment by engaging MMP12 in vivo, MMP12 was depleted by stealth siRNAs that efficiently suppressed cutaneous MMP12 levels at days 0 and 10 post-wounding (FIGS. 15a-b). Suppression of MMP12 expression in the mouse skin delayed wound healing rates when compared to those treated with a control siRNA (FIG. 16a-b). More importantly, the accelerated healing rates and the degree of re-epithelialization observed in sAgrin treated control animals were dramatically reduced upon MMP12 depletion within 10 days post-injury (FIG. 16a-b). It was further noted that increased pMLC expression in the wound edges and beds of control and sAgrin treated ones were significantly attenuated in MMP12 depleted skin sections (FIG. 16c). In addition, sAgrin induced mature and mixed collagen fiber deposition were significantly diminished upon MMP12 depletion at the wound beds (FIG. 16d). Thus, MMP12 depletion renders a mechanically incompetent environment that is majorly deprived of ECM replenishment and Acto-myosin mechanotension even when treated with sAgrin. Reduced angiogenesis (fewer blood vessels) was also observed within the wound beds of MMP12 depleted mouse skin (FIG. 16e). As such, the treatment of sAgrin failed to induce robust CD-31 expressing blood vessels when MMP12 was suppressed in the mouse skin (FIG. 16e). Together, these observations reveal that sAgrin requires MMP12 to generate a mechanically competent pro-angiogenic wound healing niche in vivo.

Agrin as Bio-Additive in Hydrogel Material Accelerates Wound Healing

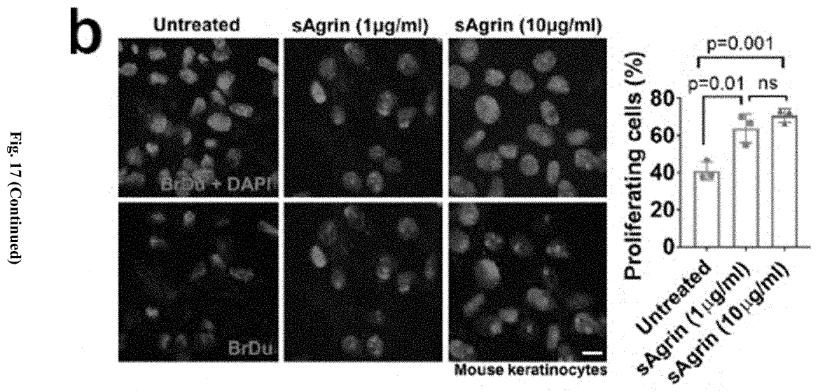

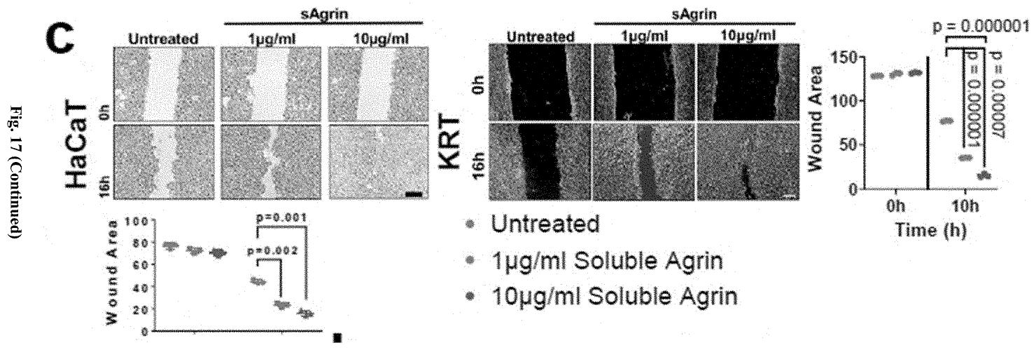

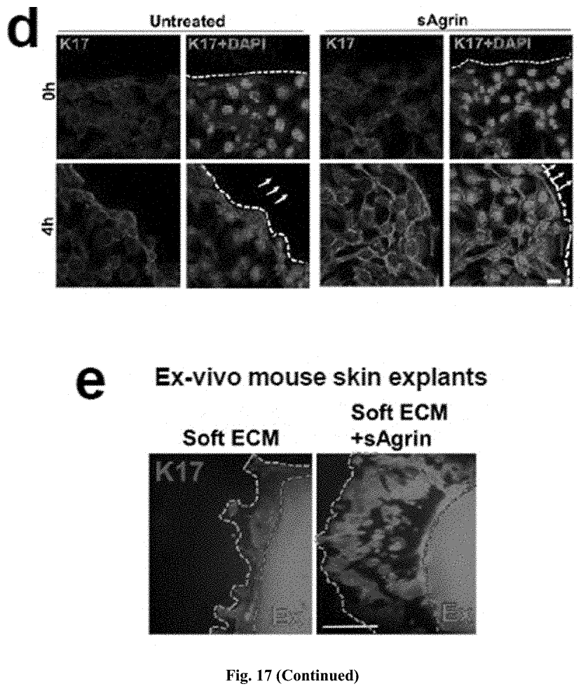

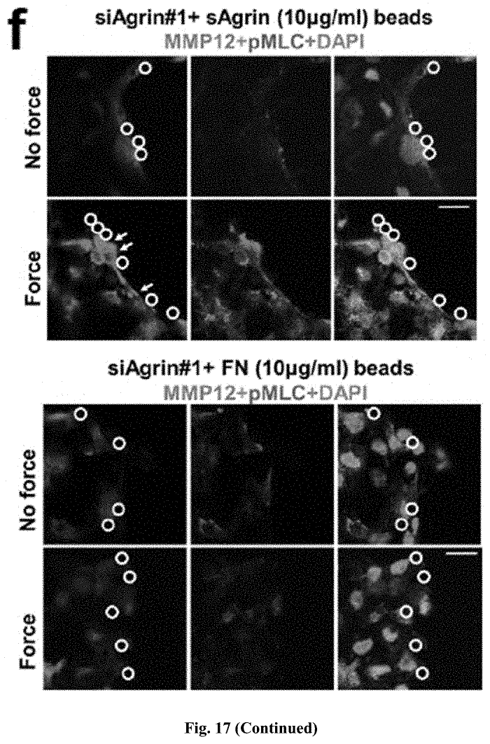

An Agrin-primed environment in wound closure prompted the inventors to test whether sAgrin, per se, accelerated in vitro and in vivo wound healing which may have clinical relevance as a wound healing biomaterial. The inventors generated sAgrin recombinant protein containing ‘z’ insert by gel-filtration that resulted in a purified protein corresponding to a size of 23 kDa (FIG. 17a). In addition to the fact that sAgrin supported the proliferation of primary mouse keratinocytes as shown by BrDu incorporation assay (FIG. 17b), accelerated migratory rates were noted when keratinocytes were treated with increasing concentrations of sAgrin (FIG. 17c). Further, sAgrin treatment also enhanced the K17 expressing leader keratinocyte migration in vitro, and ex vivo keratinocyte outgrowth from mouse skin explants, respectively (FIG. 17d-e). In Agrin depleted cells, force transmission via sAgrin (but not FN) effectively enhanced MMP12 recruitment in pMLC activated regions (FIG. 17f). These results reveal that Agrin specifically stimulates keratinocyte migration by supporting their proliferation stages.

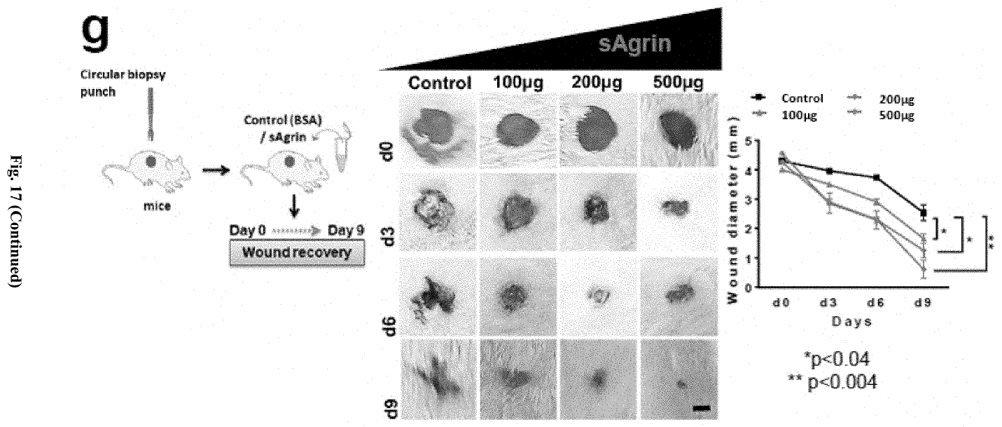

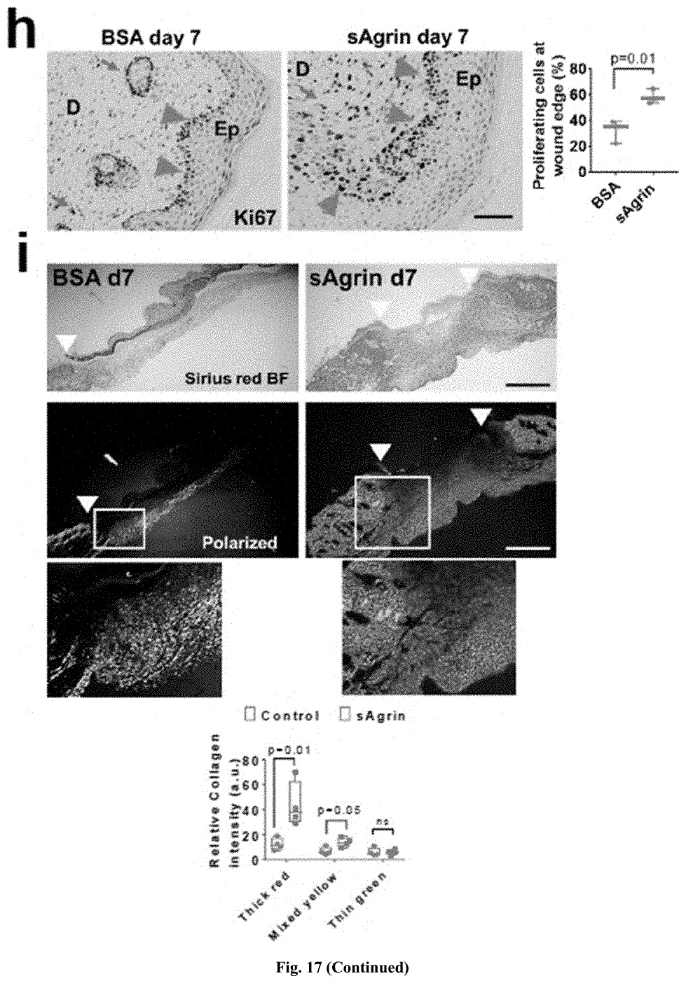

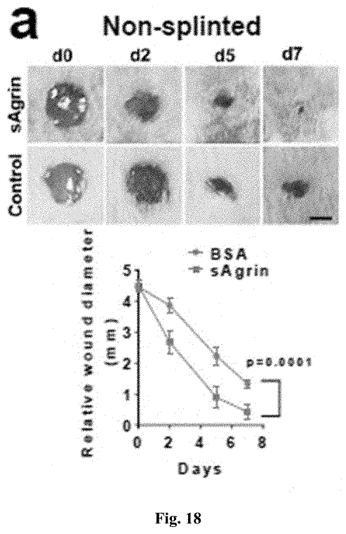

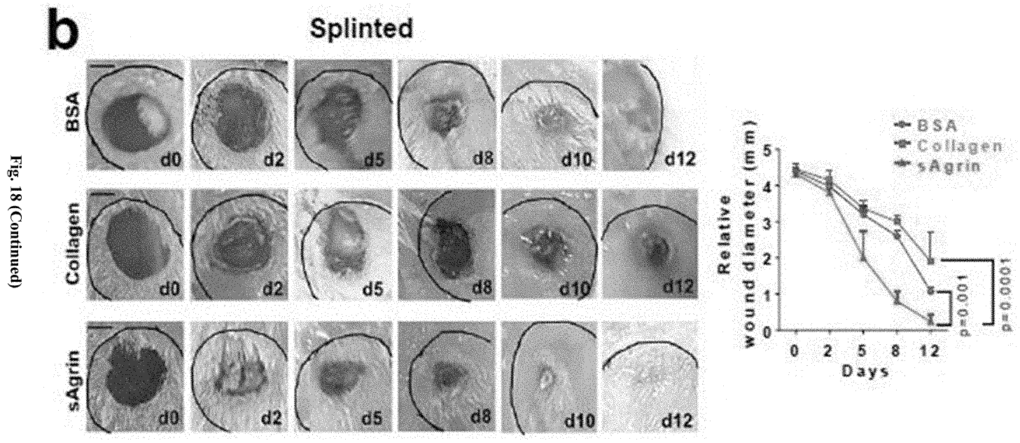

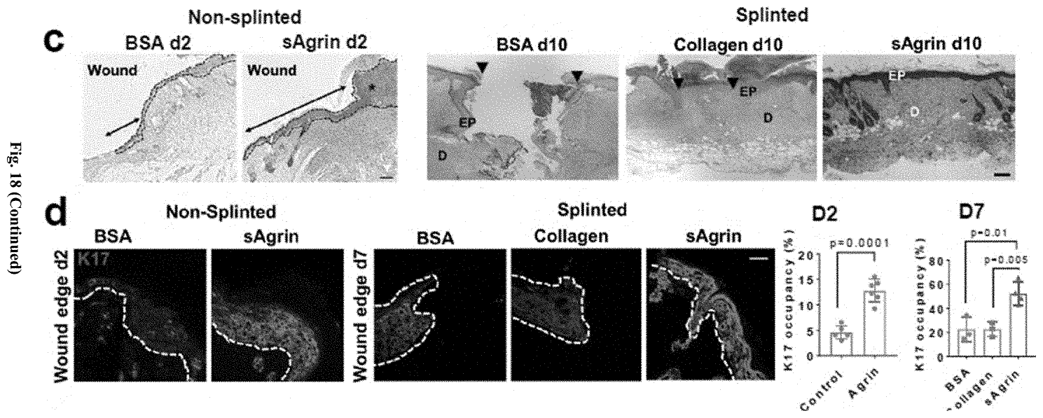

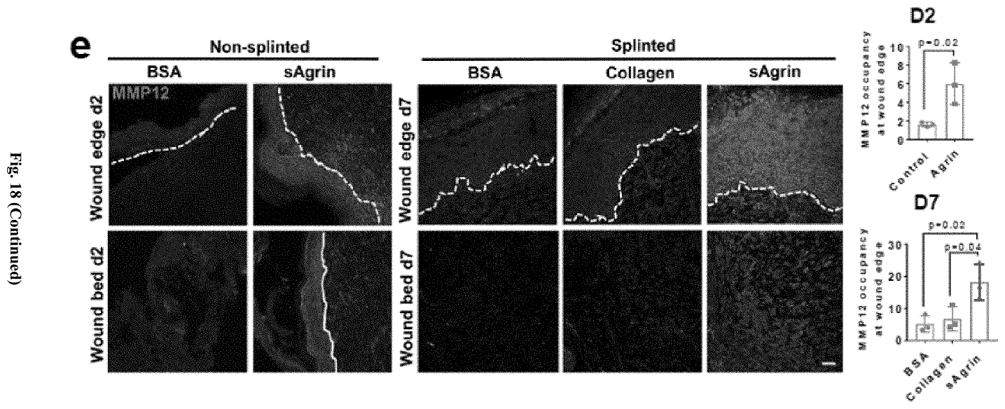

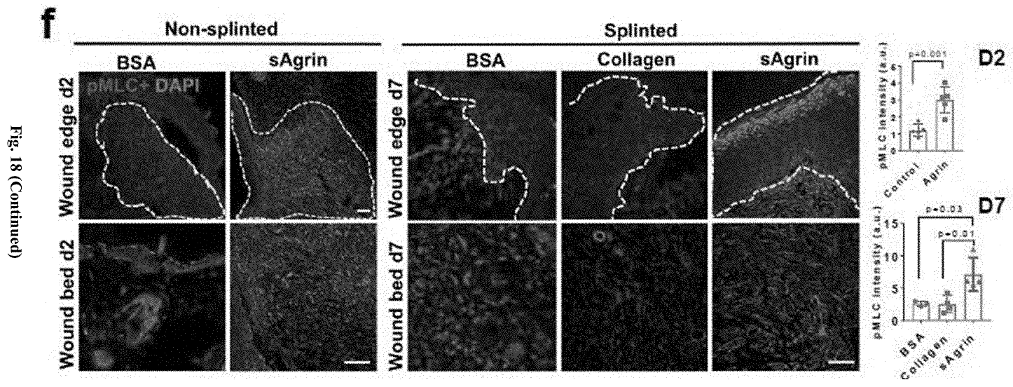

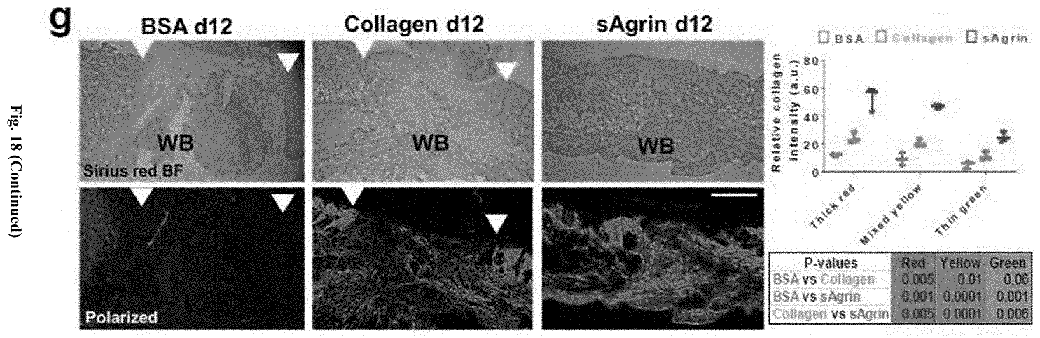

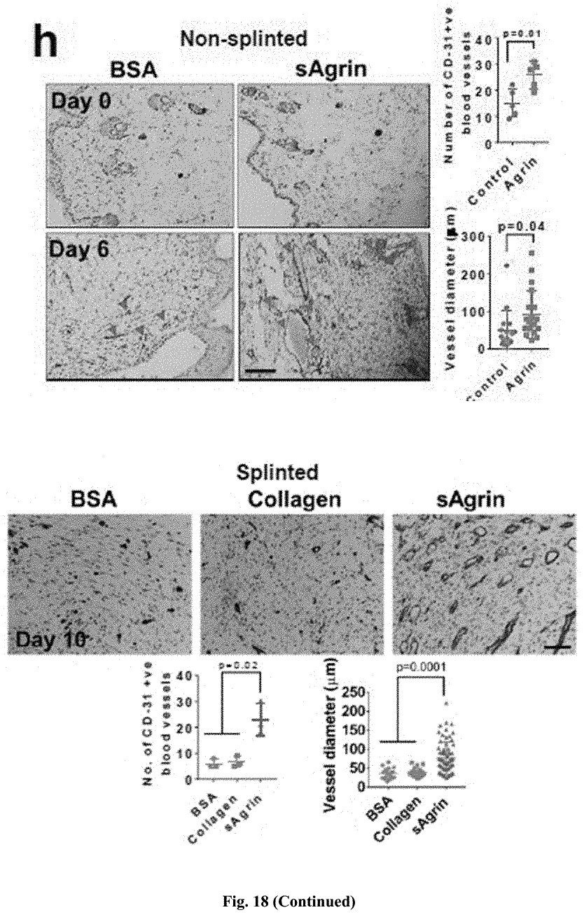

Finally, using two independent full-thickness mouse punch-biopsy non-splinted and splinted wound healing models, the wound healing attributes of sAgrin versus control proteins when supplied as bio-additive were assessed in two different hydrogels for topical application. In the first paradigm, sAgrin or a control protein (BSA) was incorporated in Vaseline, as inert hydrogel material for topical delivery to the wounded skin following a non-splinted healing model. The healing efficacy promoted by different concentrations of sAgrin incorporated in Vaseline was determined. As shown, sAgrin significantly accelerated the healing rates of mice punch wounds in a dose-dependent fashion in a non-splinted wound healing model (FIG. 17g). Subsequently, a single concentration of sAgrin (200 μg) dissolved in Pluronic F-127 as a thermoresponsive hydrogel, extensively used for topical delivery for monitoring the healing process, was tested. Likewise, when compared to the control topical hydrogel preparations, sAgrin fostered hydrogel treatment significantly boosted the rates of wound healing in vivo (FIG. 18a). The improved healing rate was supported by an enhancement of Ki67+ve proliferating cells at the base of the migrating epidermis and wound edges of sAgrin treated mouse skin sections (FIG. 17h).

In addition, the efficacy of sAgrin in comparison to rat-tail collagen-I and BSA as bio-additives was tested using splinted wound healing mouse models. The incorporation of 200 ug sAgrin within Pluronic F127 significantly accelerated wound healing when compared to similar concentrations of collagen or BSA within the observed 12 days post-injury (FIG. 18b). These results show that Agrin as a bio-additive may accelerate wound healing response irrespective of the hydrogel material used for topical delivery in two independent mouse models for wound healing. Furthermore, a significant extension of the migratory epithelial tongue was observed upon sAgrin treatment from the wound edges of non-splinted wounds (FIG. 18c). In splinted conditions at day 10 post-wounding, sAgrin treated animals predominantly represented healed skin with the emergence of regenerated hair follicles in the vicinity of the wound area in comparison to BSA or Collagen treated groups (FIG. 18c). This was accompanied by robust K17 expressing keratinocyte outgrowth, MMP12 expression and pMLC activation observed both at wound edges and the wound beds of mouse skin receiving sAgrin based hydrogels during the early phases (day 2) of wound healing in non-splinted models (FIG. 18d-f). As wound healing required a longer time-frame under splinted conditions, it was deciphered a similar increment of K17 occupancy, MMP12 expression and pMLC activity at day 7 post-injury in sAgrin treated animals in comparison to BSA or Collagen treated counterparts (FIGS. 18d-f). Since ECM replenishment within the wound bed guides the overall healing process, sAgrin treated wound beds demonstrated higher deposition of mature and mixed collagen fibers when compared to BSA and Collagen treated groups (FIG. 18g). Similar enhancement of collagen deposition by sAgrin was observed within the wound beds at day 7 in non-splinted conditions (FIG. 17i). Therefore, these shreds of data also suggest that sAgin promotes optimal collagen deposition in the wound bed to sustain the degree of stiffness within the wound healing niche (FIGS. 18g and 17i).

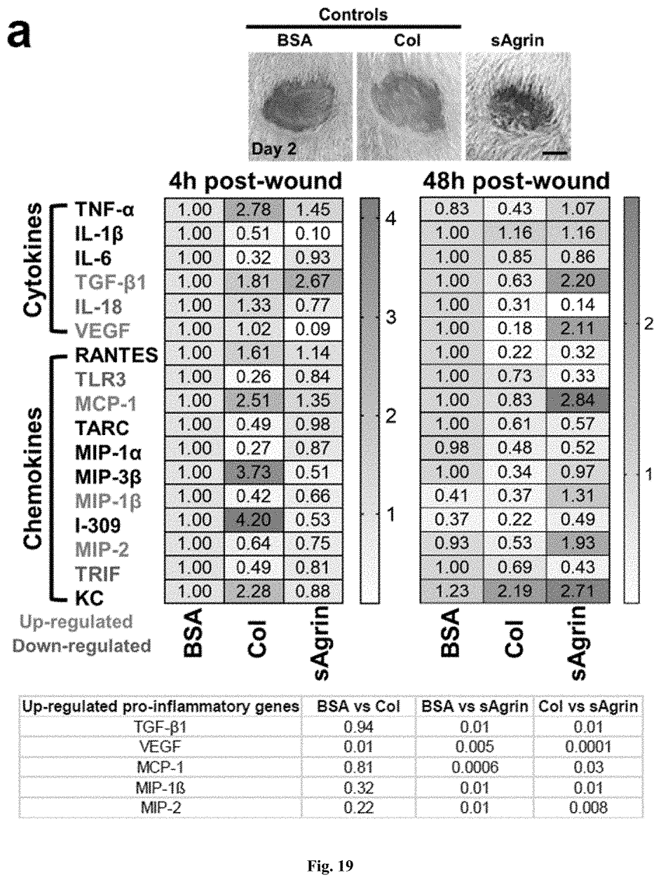

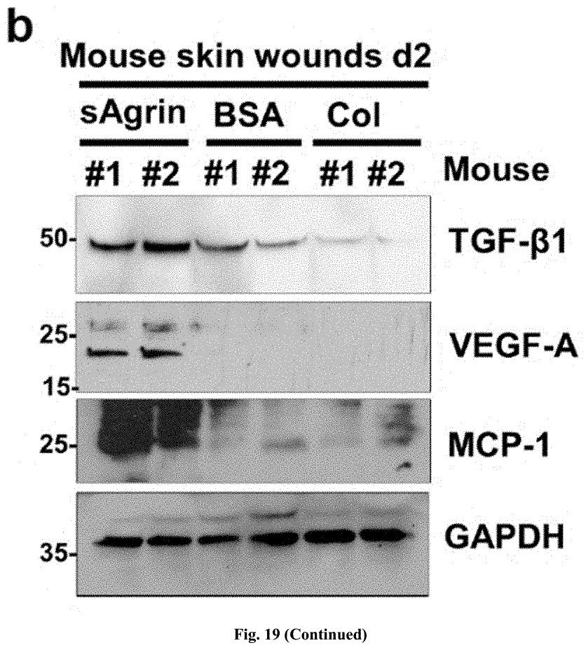

Prompt expression of inflammatory cytokines early during wound healing process is known to initiate an efficient skin healing program. Hence, it was next explored whether sAgrin selectively activated key cytokines and chemokines to modulate inflammatory responses associated with wound healing. The inventors profiled the mRNA expression of several cytokines and chemokines known to be induced within 48 hours after injury to the mouse skin. Among them, TGF-β1, VEGF-A, MCP-1, MIP-1β and MIP-2 were significantly induced in the mouse skin wounds within 48 h post-injury that received sAgrin treatment in comparison to BSA or Collagen treated groups (FIG. 19a). Indeed, augmented protein expression(s) of TGF-β1, VEGF-A and MCP-1 were confirmed in sAgrin treated groups, when compared to BSA and Collagen treated mouse skin wound tissues (FIG. 19b). Timely engagement of a highly selective set of pro-inflammatory proteins by sAgrin likely lays the foundation for an accelerated wound healing program.

To establish whether improved healing rates attributed by sAgrin occurs via sustaining angiogenesis in the wound beds would be of potential significance to the field of regenerative medicine. Consequently, on day 6 in non-splinted models, a steady enhancement in angiogenesis with an increased number of blood vessels and their respective diameters was observed in the Agrin treated mouse skin wound beds (FIG. 18h). Compared to that of BSA or Collagen, increased number of blood vessels and their diameters were also observed in sAgrin treated mouse skin wound beds at day 12 post-injury in splinted models (FIG. 18h). As such, the in vivo data suggests that Agrin may boost the interaction of dermal fibroblasts and endothelial cells, thereby enhancing angiogenic activity within the wound bed. To test this in vitro, Agrin was depleted in human dermal fibroblasts (GFP-tagged BJ) and human dermal microvascular endothelial cells (cell-tracker blue labelled HDMEC) and subjected them to a 3D co-culture sprouting angiogenesis assay (FIG. 20a). Notably, the control spheroid co-cultures generated robust sprouting which was significantly diminished by suppressing Agrin in endothelial cells and fibroblasts individually (FIG. 20b, second and third panels, respectively). Quite interestingly, depleting Agrin simultaneously in the fibroblasts and endothelial cells resulted in stronger abrogation of sprouting angiogenesis (FIG. 20b). Together, these results indicate that Agrin supports a pro-angiogenic environment in the wound bed by fostering interactions between the dermal fibroblasts and endothelial cells. Therefore, in addition to laying a solid platform for usage as biomaterial promoting skin tissue repair, the evidence in this study demonstrates that topical delivery of sAgrin accelerated in vivo wound healing by augmenting a mechanotransducing environment that sustained optimal ECM deposition, angiogenesis, and thereby promoted collective keratinocyte migration.

### DISCUSSION

Wound healing represents a complicated yet highly orchestrated biological program restoring normalcy to damaged tissue architecture. Cutaneous wound healing is sequentially characterized by a homeostasis phase where damage signals trigger clot formation to restrict blood flow, followed by an inflammatory phase that debrides wounded cells. Next, a proliferative phase governs proliferation, survival and migration of keratinocytes over the wounded area in a process termed as re-epithelialization. Timely execution of all the above culminates towards wound closure and renovation of tissue integrity. As such, a prime criterion for effective keratinocytes migration following an injury is dependent on the rate of deposition of new extracellular matrix (ECM) and its components that subsequently trigger angiogenesis that favors the healing process.

In addition to the scaffolding functions, the ECM acts as a ‘dynamic communicative layer’ to its surrounding tissue often shielding from mechanical stress and/or instructing the tissues to equate the adverse extrinsic stress, thereby sustaining tissue integrity. Besides, the skin serves as an excellent mechanoreceptor organ to analyze how mechanical forces integrate within a structured tissue architecture to sustain key biological functions. Underscoring this dynamic mechano-feedback between the skin cells and its surrounding ECM, a plethora of soluble and matrix-bound proteins are spatiotemporally regulated by the ECM. During injury, a major chunk of ECM is lost rendering the underlying tissues incapable of responding to bulk extrinsic mechanical stress, a phenomenon called mechanoperception. The lack of mechanoperceiving ability upon inflicting an injury largely accounts for large-scale deficits in actomyosin tension affecting cell survival and migration. As an appealing hypothesis favoring early phases of wound healing, the de novo expression of key ECM proteins within a wound-healing niche may recondition the underlying skin tissues by improving mechanoperception and potentially reinstating the mechanoreciprocity between the ECM and skin tissues. On this premise, an overarching interest is to identify key ECM components that integrate mechanical stimuli and establish mechanoperception within wounded cells, thereby adapting them to a wound-stressed environment.

In this study, the inventors investigated whether Agrin tunes a mechanically competent wound microenvironment enforcing skin tissue repair by improving keratinocyte mechanoperception.

Upon tissue injury, composite changes to the disorganized ECM and its surrounding tissues that integrate a collage of mechanical forces, tissue compression, bulk stress from the ECM, and collective migration of cells are required to initiate the healing program. Notably, concerted efforts to replenish the wounded ECM that sensitize the wound environment towards adverse physical stresses leading into a productive healing response and limits hypertrophic scarring are of utmost importance in regenerative medicine. Comprehensive lines of evidence presented here uncovers several important findings that advance our understanding of mechanobiology in wound healing and offer immense significance to tissue repair strategies. Firstly, Agrin represents a vital ECM proteoglycan whose expression is triggered in the wounded skin tissue and this Agrin-enriched microenvironment tunes the mechanical landscape for productive wound healing (FIG. 12g). Secondly, Agrin guides collective keratinocytes migration over the wounded sites by sensitizing them towards different forms of physical parameters such as bulk ECM rigidity, mechanical force, and geometrical constraints. Thirdly, the mechanoperception ability conferred by Agrin robustly overhauls the cytoskeletal architecture following wound injury and is largely dependent on MMP12 activation. Thus, Agrin mechanotransduction via MMP12 activation provides an integrated mechanism that empowers wounded skin cells to retaliate adverse mechanical stress caused by wound injury and accelerate their migration rates. Fourthly, appreciating the importance of a mechanically competent Agrin enriched wound environment, the results in this study additionally reveal that appropriate utilization of sAgrin as a bio-additive wound healing material may offer great clinical value for wounds.

Epithelial cells experience a wide array of physical stresses that include ECM rigidity, topographic changes in cell shape and geometry, lack of adhesion, and application of mechanical forces. Each of these parameters dictates cell behavior in a wound environment, however, the nature of ECM proteins that enable wounded cells to respond to such physical parameters are less known. Driving this conviction, Agrin, in part, sensitized keratinocytes to enforce collective migration in response to several physical parameters including bulk ECM rigidity, shift cytoskeletal tension upwards in geometrically constrained architectures, and act as localized mechanotransducer when extrinsic forces are applied to wounded cells (FIG. 21). Nonetheless, since FN engagement of integrins failed to restore actomyosin cables in the absence of Agrin upon extrinsic force, the magnitude of cytoskeletal overhauling and enhanced mechanoperception in response to a wound microenvironment appears to be unique for Agrin mechanotransduction. Consistent with the notion that collective cell migration towards a stiffened ECM generates higher traction forces, the potential of Agrin as an ECM player that bestows a considerable degree of elasticity amongst keratinocytes aiding them with higher traction forces, cytoskeletal tension, and improved motility post-injury is of significance. While Agrin depletion independently inhibited 2D scratch wound healing in keratinocytes and dermal fibroblasts, 3D stiffness-dependent keratinocyte fibroblast co-culture migration assays revealed that dermal fibroblasts sourced Agrin is insufficient to restore the migration of Agrin depleted keratinocytes on stiff substrates. Also, the knockdown of Agrin in the dermal fibroblasts did not impact the migratory rates of overlaid keratinocytes with intact Agrin on stiff substrates. Given that non-wounded and wounded keratinocytes expressed higher Agrin than in dermal fibroblasts, these lines of investigations advocate that Agrin expressed by keratinocytes acts as a master regulator coordinating migration over wounded regions upon sensing bulk ECM rigidity.

The dynamic ECM reorganization of an Agrin-enriched environment-induced shortly after wounding remains yet to be fully established. The transcriptomic data in this study reveals an overall loss of collagens and ECM structural components in Agrin depleted keratinocytes. Given that Agrin depletion delays wound healing, it is evident that Agrin confers comprehensive ECM reorganization signals via MMP12 to sustain wound healing.

1. A pharmaceutical composition comprising an Agrin fragment or derivative thereof, wherein the Agrin fragment or derivative thereof comprises the LG3 domain of Agrin and an eight-amino-acid insert ELANEIPV (SEQ ID NO: 1) at the z-site of the LG3 domain.

2. The pharmaceutical composition of claim 1, wherein the LG3 domain of Agrin without any insert at the z-site comprises the sequence
(SEQ ID NO: 5)
EYLNAVTESEKALQSNHFELSLRTEATQGLVLWSGKATERADYVALAIV
DGHLQLSYNLGSQPVVLRSTVPVNTNRWLRVVAHREQREGSLQVGNEAP
VTGSSPLGATQLDTDGALWLGGLPELPVGPALPKAYGTGFVGCLRDVVV
GRHPLHLLEDAVTKPELRPC.

3. The pharmaceutical composition of claim 1, wherein the Agrin fragment or derivative thereof comprises the sequence
(SEQ ID NO: 7)
DTLAFDGRTFVEYLNAVTESELANEIPVEKALQSNHFELSLRTEATQGL
VLWSGKATERADYVALAIVDGHLQLSYNLGSQPVVLRSTVPVNTNRWLR
VVAHREQREGSLQVGNEAPVTGSSPLGATQLDTDGALWLGGLPELPVGP
ALPKAYGTGFVGCLRDVVVGRHPLHLLEDAVTKPELRPCPTP.

4. The pharmaceutical composition of claim 1, wherein the Agrin fragment or derivative further comprises the LG2 domain of Agrin.

5. The pharmaceutical composition of claim 4, wherein the LG2 domain of Agrin without any insert at the y-site comprises the sequence
(SEQ ID NO: 2)
PFLADFNGFSHLELRGLHTFARDLGEKMALEVVFLARGPSGLLLYNGQK
TDGKGDFVSLALRDRRLEFRYDLGKGAAVIRSREPVTLGAWTRVSLERN
GRKGALRVGDGPRVLGESPVPHTVLNLKEPLYVGGAPDFSKLARAAAVS
SGFDGAIQLVSLGGRQLLTPEHVLRQVDVTSFAGHPC.

6. The pharmaceutical composition of claim 4, wherein the Agrin fragment or derivative thereof comprises the sequence
(SEQ ID NO: 8)
LGREGTFCQTASGQDGSGPFLADFNGFSHLELRGLHTFARDLGEKMALE
VVFLARGPSGLLLYNGQKTDGKGDFVSLALRDRRLEFRYDLGKGAAVIR
SREPVTLGAWTRVSLERNGRKGALRVGDGPRVLGESPVPHTVLNLKEPL
YVGGAPDFSKLARAAAVSSGFDGAIQLVSLGGRQLLTPEHVLRQVDVTS
FAGHPCTRASGHPCLNGASCVPREAAYVCLCPGGFSGPHCEKGLVEKSA
GDVDTLAFDGRTFVEYLNAVTESELANEIPVEKALQSNHFELSLRTEAT
QGLVLWSGKATERADYVALAIVDGHLQLSYNLGSQPVVLRSTVPVNTNR
WLRVVAHREQREGSLQVGNEAPVTGSSPLGATQLDTDGALWLGGLPELP
VGPALPKAYGTGFVGCLRDVVVGRHPLHLLEDAVTKPELRPCPTP.

7. The pharmaceutical composition of claim 1, wherein the Agrin fragment or derivative thereof is soluble.

8. A vector comprising the nucleic acid molecule encoding for an Agrin fragment or derivative thereof, wherein the Agrin fragment or derivative thereof comprises the LG3 domain of Agrin and an eight-amino-acid insert ELANEIPV (SEQ ID NO: 1) at the z-site of the LG3 domain.

9. A host cell comprising the vector of claim 8.

10. A hydrogel or scaffold comprising the pharmaceutical composition of claim 1.

11. The hydrogel of claim 10, wherein the hydrogel is an inert hydrogel or a thermoresponsive hydrogel.

12. (canceled)

13. A method of treating a wound, the method comprising administering a pharmaceutically effective amount of the pharmaceutical composition of claim 1 to a subject in need thereof.

14. (canceled)

15. The method of claim 13, wherein the wound is a slow healing wound or a non-healing wound.
